(19) United States
(12) Patent Application Publication
(10) Pub. No.: US 2025/0002601 A1
(43) Pub. Date: Jan. 2, 2025

(54) MULTISPECIFIC ANTIBODIES BINDING TO CD20, NKP46, CD16 AND CONJUGATED TO IL-2

(71) Applicant: INNATE PHARMA, MARSEILLE (FR)

(72) Inventors: LAURENT GAUTHIER, MARSEILLE (FR); YANNIS MOREL, MARSEILLE (FR); OLIVIER DEMARIA, MARSEILLE (FR); BENJAMIN ROSSI, MARSEILLE (FR)

(21) Appl. No.: 18/568,806

(22) PCT Filed: Jun. 8, 2022
(86) PCT No.: PCT/EP2022/065514

§ 371 Date: Dec. 9, 2023

Related U.S. Application Data
(60) Provisional application No. 63/208,514, filed on Jun. 9, 2021.

(51) Int. Cl. C07K 16/28; A61K 39/00; A61P 35/00; C07K 14/55

(52) U.S. Cl. CPC C07K 16/2887; A61P 35/00; C07K 14/55; C07K 16/283; C07K 16/2866; A61K 2039/505; C07K 2317/31; C07K 2317/522; C07K 2317/524; C07K 2317/526; C07K 2317/53; C07K 2317/55; C07K 2317/565; C07K 2317/622; C07K 2317/73; C07K 2317/92; C07K 2317/94; C07K 2319/30

(57) ABSTRACT

The present invention relates to multispecific protein that binds to human NKp46, human CD20, human CD122, and optionally human CD16A. The protein according to the invention have utility in the treatment of disease, such as cancer.

### CROSS-REFERENCE TO RELATED APPLICATIONS

This application is the U.S. national stage application of International Application No. PCT/EP2022/065514, filed Jun. 8, 2022, which claims the benefit of U.S. Provisional Application No. 63/208,514 filed on 9 Jun. 2021, the disclosure of which is incorporated herein by reference in its entirety; including any drawings and sequence listings.

### REFERENCE TO THE SEQUENCE LISTING

The present application is being filed along with a Sequence Listing in electronic format. The Sequence Listing is provided as a file entitled “Seq-List.txt” “Seq-List-replace.txt”, created Jul. 5, 2024, which is 188,833 bytes in size. The information in the electronic format of the Sequence Listing is incorporated herein by reference in its entirety.

### FIELD OF THE INVENTION

The disclosure relates to multispecific binding proteins comprising a first and a second antigen binding domains (ABDs), a cytokine moiety, and all or part of an immunoglobulin Fc region or variant thereof, wherein the first ABD binds specifically to human CD20 and the second ABD bind specifically to human NKp46 and optionally wherein all or part of the immunoglobulin Fc region or variant thereof binds to human CD16. Multispecific binding proteins of the disclosure can advantageously redirect effector cells to lyse CD20-expressing cells of interest via multiple receptors. The disclosure also relates to methods for making said binding proteins, compositions thereof, and their uses including the treatment of diseases, including diseases involving CD20-expressing cells.

### BACKGROUND

Natural killer (NK) cells are a subpopulation of lymphocytes that are involved in non-conventional immunity. NK cells provide an efficient immunosurveillance mechanism by which undesired cells such as tumor or virally-infected cells can be eliminated. Characteristics and biological properties of NK cells include the expression of surface antigens including CD16, CD56 and/or CD57, the absence of the α/β or γ/δ TCR complex on the cell surface, the ability to bind to and kill cells in a MHC-unrestrictive manner and in particular cells that fail to express “self” MHC/HLA antigens by the activation of specific cytolytic enzymes, the ability to kill tumor cells or other diseased cells that express a ligand for NK activating receptors, and the ability to release protein molecules called cytokines that stimulate the immune response.

Interest has also focused on natural killer (NK) cells due to their potential anti-tumor properties. WO2017114694 reports variable regions for NKp46 binding proteins, for the production of multispecific proteins with the ability to specifically redirect NK cells to lyse a target cell of interest. However, NK cells have been shown to cause toxicity in mice through their hyper-activation and secretion of multiple inflammatory cytokines when IL-2 was administered together with IFN-a (Rothschilds et al, Oncoimmunology. 2019; 8 (5):). In addition, NK cells were also shown to cause toxicity of the cytokine IL-15 that also signals through IL-2Rβγ (see WO2020247843 citing Guo et al, J Immunol. 2015; 195(5):2353-64).

One potential solution to the immune toxicity mediated by cytokines such as IL-2 was to fuse it to or associate it with a tumor-specific antibody. However, it was found that while IL-2 indeed synergized with antitumor antibody in anti-tumor effect in vivo, the inclusion of IL-2 and anti-tumor antigen antibody in the same molecule presented no efficacy or toxicity advantage. The IL-2 moiety entirely governed biodistribution, explaining the observation that immunocytokines recognizing irrelevant antigen performed equivalently to tumor-specific immunocytokines when combined with antibody (Tzeng et al. Proc Natl Acad Sci USA. 2015 Mar. 17; 112(11): 3320-332).

Studies focusing on the effect of cytokines on NK cells have generally focused on single cytokines or simple combinations. More recently, it has been reported that IL-15, IL-18, IL-21, and IFN-α, alone and in combination, and their potential to synergize with IL-2, and that very low concentrations of both innate and adaptive common γ chain cytokines synergize with equally low concentrations of IL-18 to drive rapid and potent NK cell CD25 and IFN-γ expression (Nielsen et al. Front Immunol. 2016; 7: 101). However, administration of cytokines to humans has involved toxicity, which makes combination treatment with cytokines challenging. Furthermore, little remains known on potential synergies or interaction between cytokine receptor signaling pathways and other activating receptors in NK cells. There is therefore a need for new ways to mobilize NK cells in the treatment of disease, particularly cancer.

Still, there is an urgent need for active agents for treating or preventing proliferative disorders such as CD20 positive B-non Hodgkin lymphoma (NHL), diffuse large B-cell lymphoma (DLBCL), mantle cell lymphoma (MCL), follicular lymphoma (FL), chronic lymphocytic leukaemia (CLL) or myelodysplastic syndromes (MDS).

There is also a need for novel NK engagers with a therapeutic effect.

There is also a need for novel compounds which are easier to manufacture and/or administer, with no or decreased side-effects. In particular, there is a need for novel compounds with no or decreased risk of cytokine release syndrome in patients.

NK cells have the potential to mediate anti-tumor immunity.

### SUMMARY OF THE INVENTION

The present invention arises from the discovery of functional multispecific binding proteins that bind to NKp46 and a cytokine receptor (e.g. CD122) on NK cells, optionally that further bind CD16A on NK cells, and that also bind to the tumoral antigen CD20 on a target cell, where in the multi-specific proteins are capable of increasing NK cell cytotoxicity toward a target cell that expresses the antigen of interest (e.g., a cell that contributes to disease, a cancer cell).

In one embodiment, the disclosure relates to a polypeptide comprising the amino acid sequence of SEQ ID NO: 1, or an amino acid sequence having at least 90%, 95% or 98% of sequence identity therewith. The polypeptide can associate with (e.g. dimerize or combine with) one or two further polypeptides to form a binding protein (e.g. a multimeric binding protein) that binds specifically to human CD20, human NKp46, human CD122, and optionally human CD16. Also provided are multimeric (e.g. dimeric, trimeric) binding proteins comprising such one two or three polypeptide(s) or polypeptide chain(s), as well as methods of producing a multimeric (e.g. dimeric, trimeric) binding proteins.

In one embodiment, the disclosure relates to a binding protein (e.g. a multimeric protein) that binds specifically to human CD20, human NKp46, human CD122, and optionally human CD16, wherein said protein comprises a first (I) polypeptide having the amino acid sequence of SEQ ID NO: 1, and a second (II) polypeptide having the amino acid sequence of SEQ ID NO: 70.

In one embodiment, the disclosure relates to a multimeric binding protein that binds specifically to human CD20, human NKp46, human CD122, and optionally human CD16, wherein said protein comprises a first (I) polypeptide chain having the amino acid sequence of SEQ ID NO: 1, a second (II) polypeptide chain having the amino acid sequence of SEQ ID NO: 9, and a third (III) polypeptide chain having the amino acid sequence of SEQ ID NO: 17.

In one embodiment, the disclosure relates to a multimeric binding protein that binds specifically to human CD20, human NKp46, human CD122, and optionally human CD16, wherein said protein comprises a first (I) polypeptide chain having the amino acid sequence of SEQ ID NO: 1, a second (II) polypeptide chain having the amino acid sequence of SEQ ID NO: 73, and a third (III) polypeptide chain having the amino acid sequence of SEQ ID NO: 74.

In one embodiment, the disclosure relates to a multimeric binding protein that binds specifically to human CD20, human NKp46, human CD122, and optionally human CD16, wherein said protein comprises a first (I) polypeptide chain having the amino acid sequence of SEQ ID NO: 66, a second (II) polypeptide chain having the amino acid sequence of SEQ ID NO: 67, and a third (III) polypeptide chain having the amino acid sequence of SEQ ID NO: 17.

In one embodiment, provided is binding protein (e.g. a multimeric protein of the disclosure) comprising a first (I) polypeptide having an amino acid sequence having at least 90% of sequence identity with the amino acid sequence of SEQ ID NOS: 1 or 66, a second (II) polypeptide having an amino acid sequence having at least 90% of sequence identity with the amino acid sequence of SEQ ID NOS: 6, 67, 70, or 73, and optionally a third (III) polypeptide having an amino acid sequence having at least 90% of sequence identity with the amino acid sequence of SEQ ID NOS: 17 or 74.

In one embodiment, provided is a multimeric binding protein (e.g. a protein of the disclosure) comprising a first and a second antigen binding domain (ABDs) that comprises an immunoglobulin heavy variable domain (VH) and an immunoglobulin light chain variable domain (VL), wherein each VH and VL comprises three complementary determining regions (CDR1, CDR2, and CDR3); and wherein
(i) the first antigen binding domain (ABD) specifically binds to human CD20 and comprises:
   a VH1 comprising a CDR1, CDR2 and CDR3 corresponding to the amino acid sequences of SEQ ID NO: 29 (HCDR1), SEQ ID NO: 32 (HCDR2), SEQ ID NO: 35 (HCDR3), and
   a VL1 comprising a CDR1, CDR2 and CDR3 corresponding to the amino acid sequences of SEQ ID NO: 38 (LCDR1), SEQ ID NO: 41 (LCDR2), SEQ ID NO: 44 (LCDR3);
(ii) the second antigen binding domain (ABD) specifically binds to human NKp46, and comprises:
   a VH2 comprising a CDR1, CDR2 and CDR3 corresponding to the amino acid sequences of SEQ ID NO: 47 (HCDR1), SEQ ID NO: 50 (HCDR2), SEQ ID NO: 53 (HCDR3), and
   a VL2 comprising a CDR1, CDR2 and CDR3 corresponding to the amino acid sequences of SEQ ID NO: 56 (LCDR1), SEQ ID NO: 59 (LCDR2), SEQ ID NO: 62 (LCDR3).

In a particular embodiment, the multimeric binding protein according to the disclosure comprises a variant IL-2 polypeptide, said variant IL-2 comprising an amino acid sequence of SEQ ID NO: 65.

In a particular embodiment, the multimeric binding protein of the disclosure comprises all or part of an immunoglobulin Fc region or variant thereof that binds to a human Fc-γ receptor, said all of part of an immunoglobulin Fc region comprising an CH2-CH3 domain having at least 90% of sequence identity with an amino acid sequence of SEQ ID NO: 6 or 14.

Also provided is a binding protein comprising a first and a second antigen binding domains (ABDs), a cytokine moiety and all or part of an immunoglobulin Fc region or variant thereof, wherein the first ABD has a Fab structure and comprises an immunoglobulin heavy chain (VH) and an immunoglobulin light chain variable domain (VL), wherein each VH and VL comprises three complementary determining regions (CDR1, CDR2, CDR3); and wherein:
(i) the first ABD binds specifically to human CD20 and comprises:
   a VH1 comprising a CDR1, CDR2 and CDR3 corresponding to the amino acid sequences of SEQ ID NO: 29 (HCDR1), SEQ ID NO: 32 (HCDR2), SEQ ID NO: 35 (HCDR3), and
   a VL1 comprising a CDR1, CDR2 and CDR3 corresponding to the amino acid sequences of SEQ ID NO: 38 (LCDR1), SEQ ID NO: 41 (LCDR2), SEQ ID NO: 44 (LCDR3);
(ii) the second ABD binds specifically to human NKp46 and comprises:
   a VH2 comprising a CDR1, CDR2 and CDR3 corresponding to the amino acid sequences of SEQ ID NO: 47 (HCDR1), SEQ ID NO: 50 (HCDR2), SEQ ID NO: 53 (HCDR3), and
   a VL2 comprising a CDR1, CDR2 and CDR3 corresponding to the amino acid sequences of SEQ ID NO: 56 (LCDR1), SEQ ID NO: 59 (LCDR2), SEQ ID NO: 62 (LCDR3);
and wherein all or part of the immunoglobulin Fc region or variant thereof binds to a human Fc-γ receptor.

In one embodiment, the cytokine moiety is a variant IL-2.

In one embodiment, the first and second ABDs of the binding protein have a Fab structure. In one embodiment, the first ABD of the binding protein has a Fab structure and the second ABD of the binding protein has an scFv structure.

In one embodiment, the binding protein according to the disclosure comprises three polypeptide chains (I), (II) and (III) that form the two ABDs as defined above:

V_{1A}-C_{1A}-Hinge₁-(Fc domain)_{A} (I)

V_{1B}-C_{1B}-Hinge₂-(Fc domain)_{B}-L₁-V_{2A}-C_{2A} (II)

V_{2B}-C_{2B}-Hinge₃-L₂-IL-2 (III)
wherein:
V_{1A} and V_{1B} form a binding pair V₁ (V_{H1}/V_{L1}) of the first ABD;
V_{2A} and V_{2B} form a binding pair V₂ (V_{H2}/V_{L2}) of the second ABD;
C_{1A} and C_{1B} form a pair C₁ (CH1/C_{L}) and C_{2A} and C_{2B} form a pair C₂ (CH1/C_{L}) wherein CH1 is an immunoglobulin heavy chain constant domain 1 and C_{L} is an immunoglobulin light chain constant domain;
Hinge₁, Hinge₂ and Hinge₃ are identical or different and correspond to all or part of an immunoglobulin hinge region;
(Fc domain)_{A} and (Fc domain)_{B} are identical or different, and comprise a CH2-CH3 domain;
L₁ and L₂ are an amino acid linker, wherein L₁ and L₂ can be different or the same;
IL-2 is a variant human interleukin-2 polypeptide or portion thereof that binds to CD122 present on NK cells.

In another embodiment, the binding protein according to the disclosure comprises two polypeptide chains (I) and (II) that form two ABDs as defined above:

V_{1A}-C_{1A}-Hinge₁-(Fc domain)_{A} (I)

V_{1B}-C_{1B}-Hinge₂-(Fc domain)_{B}-L₁-V_{2A}-L₂-V_{2B}-L₃-IL-2 (II)
wherein:
V_{1A} and V_{1B} form a binding pair V₁ (V_{H1}/V_{L1}) of the first ABD;
V_{2A} and V_{2B} form a binding pair V₂ (V_{H2}/V_{L2}) of the second ABD;
C_{1A} and C_{1B} form a pair C₁ (CH1/C_{L}) wherein CH1 is an immunoglobulin heavy chain constant domain 1 and C_{L} is an immunoglobulin light chain constant domain;
Hinge₁ and Hinge₂ are identical or different and correspond to all or part of an immunoglobulin hinge region;
(Fc domain)_{A} and (Fc domain)_{B} are identical or different, and comprise a CH2-CH3 domain;
L₁, L₂ and L₃ are an amino acid linker, wherein L₁, L₂ and L₃ can be different or the same;
IL-2 is a variant human interleukin-2 polypeptide or portion thereof that binds to CD122 present on NK cells.

In one embodiment, the CH1 domain of the binding protein of the disclosure is an immunoglobulin heavy chain constant domain 1 that comprises the amino acid sequence of SEQ ID NO: 12.

In one embodiment, the C_{K} domain of the binding protein of the disclosure is an immunoglobulin kappa light chain constant domain (C_{K}) that comprises the amino acid sequence of SEQ ID NO: 4.

In one embodiment, the (Fc domain)_{A} of the binding protein of the disclosure comprises a CH2-CH3 domains corresponding to the amino acid sequence of SEQ ID NO: 6.

In one embodiment, the (Fc domain)_{B} of the binding protein of the disclosure comprises a CH2-CH3 domains corresponding to the amino acid sequence of SEQ ID NO: 14.

In one embodiment, the Hinge₁ domain of the binding protein of the disclosure has an amino acid sequence of SEQ ID NO: 5.

In one embodiment, the Hinge₂ domain of the binding protein of the disclosure has an amino acid sequence of SEQ ID NO: 13.

In one embodiment, the Hinge₃ domain of the binding protein of the disclosure has an amino acid sequence of SEQ ID NO: 19.

In one embodiment, the linker L₁ of the binding protein of the disclosure has an amino acid sequence of SEQ ID NO: 15.

In one embodiment, the linker L₂ of the binging protein of the disclosure has an amino acid sequence of any one of SEQ ID NOS: 20-23.

In a particular embodiment, the binding protein of the disclosure has a residue N297 of the Fc domain or variant thereof according to Kabat numbering that comprises a N-linked glyclosylation. Preferably, the Fc domain or variant thereof of the binding protein of the disclosure binds to a human CD16A (FcγRIII) polypeptide.

In one embodiment, the binding protein of the disclosure comprises at least two polypeptide chains linked by at least one disulfide bridge. Preferably, the polypeptide chains (I) and (II) of the binding protein of the disclosure are linked by one disulfide bridge between C_{1A} and Hinge₂, two disulfide bridges between Hinge₁ and Hinge₂ and wherein the polypeptide chains (II) and (III) are linked by one disulfide bridge between Hinge₃ and C_{2B}.

In one embodiment, the V_{1A} domain of the binding protein of the disclosure is V_{L1} and V_{1B} domain is V_{H1}.

In one embodiment, the V_{2A} domain of the binding protein of the disclosure is V_{H2} and V_{2B} domain is V_{L2}.

In one embodiment, the C_{1A} domain of the binding protein of the disclosure is C_{K} and C_{1B} domain is CH1.

In one embodiment, the C_{2A} domain of the binding protein of the disclosure is C_{K} and C_{2B} domain is CH1.

In an alternative embodiment, the C_{2A} domain of the binding protein of the disclosure is CH1 and C_{2B} domain is C_{K}.

In one embodiment, the binding protein of the disclosure comprises:
(a) V_{H1} and V_{L1} corresponds to the amino acid sequences of SEQ ID NO: 11 and 3 respectively,
and/or
(b) V_{H2} and V_{L2} corresponds to the amino acid sequences of SEQ ID NO: 93 and 95 respectively.

In one embodiment, the variant IL-2 of the binding protein of the disclosure displays reduced binding to CD25 compared to a wild-type human IL-2 polypeptide.

In one embodiment, the binding variant IL-2 of the binding protein of the disclosure comprises an amino acid sequence at least 90% identical to a sequence selected from SEQ ID NOS: 24-28 and 65, or to a contiguous sequence of at least 40, 50, 60, 70, 80 or 100 amino acid residues thereof.

In one embodiment, the binding protein of the disclosure comprises:
A polypeptide (I) consisting of an amino acid sequence of SEQ ID NO: 1;
A polypeptide (II) consisting of an amino acid sequence of SEQ ID NO: 9; and
A polypeptide (III) consisting of an amino acid sequence of SEQ ID NO: 17.

In an alternative embodiment, the binding protein of the disclosure comprises:
A polypeptide (I) consisting of an amino acid sequence of SEQ ID NO: 1;
A polypeptide (II) consisting of an amino acid sequence of SEQ ID NO: 73; and
A polypeptide (III) consisting of an amino acid sequence of SEQ ID NO: 74.

In one embodiment, the Fc domains of the binding protein of the disclosure comprises N297 residue (according to Kabat numbering) mutated to prevent said residue to be glycosylated. Preferably, said mutation is a N297S substitution. In a preferred embodiment, such mutation substantially abolish CD16A binding of the binding protein of the disclosure.

In one embodiment, the binding protein of the disclosure comprises:
A polypeptide (I) consisting of an amino acid sequence of SEQ ID NO: 66;
A polypeptide (II) consisting of an amino acid sequence of SEQ ID NO: 67; and
A polypeptide (III) consisting of an amino acid sequence of SEQ ID NO: 17.

In an alternative embodiment, the binding protein of the disclosure comprises:
A polypeptide (I) consisting of an amino acid sequence of SEQ ID NO: 66;
A polypeptide (II) consisting of an amino acid sequence of SEQ ID NO: 75; and
A polypeptide (III) consisting of an amino acid sequence of SEQ ID NO: 74.

In another embodiment, the Fc domains of the binding protein of the disclosure, L234A, L235E, G237A, A330S and/or P331S substitutions according to kabat numbering.

Accordingly, one binding protein of the disclosure comprises:
A polypeptide (I) consisting of an amino acid sequence of SEQ ID NO: 68;
A polypeptide (II) consisting of an amino acid sequence of SEQ ID NO: 69; and
A polypeptide (III) consisting of an amino acid sequence of SEQ ID NO: 17.

In an alternative embodiment, the binding protein of the disclosure comprises:
A polypeptide (I) consisting of an amino acid sequence of SEQ ID NO: 68;
A polypeptide (II) consisting of an amino acid sequence of SEQ ID NO: 76; and
A polypeptide (III) consisting of an amino acid sequence of SEQ ID NO: 74.

In an alternative embodiment, the first ABD of the binding protein of the disclosure that binds to CD20 is an Fab and the second ABD that binds to NKp46 is an scFv.

In an alternative embodiment, the first ABD of the binding protein of the disclosure is a VH/VL pair.

In one embodiment, the binding protein of the disclosure comprises:
A polypeptide (I) consisting of an amino acid sequence of SEQ ID NO: 77;
A polypeptide (II) consisting of an amino acid sequence of SEQ ID NO: 78; and
A polypeptide (III) consisting of an amino acid sequence of SEQ ID NO: 74.

In an alternative embodiment, the binding protein of the disclosure comprises:
A polypeptide (I) consisting of an amino acid sequence of SEQ ID NO: 77;
A polypeptide (II) consisting of an amino acid sequence of SEQ ID NO: 79; and
A polypeptide (III) consisting of an amino acid sequence of SEQ ID NO: 17.

In one embodiment, the second ABD of the binding protein of the disclosure and the cytokine moiety have an arrangement;

-L1-V_{2A}-L2-V_{2B}-L3-IL-2,
Wherein V_{2A} and V_{2B} form a binding pair V₂ (V_{H2}/V_{L2}) of the second ABD;
L₁, L₂ and L₃ are an amino acid linker, wherein L₁, L₂ and L₃ can be different or the same;
IL-2 is a variant human interleukin-2 polypeptide or portion thereof that binds to CD122 present on NK cells.

In one embodiment, the V_{2A} domain of a binding protein of the disclosure is V_{H2} and V_{2B} domain is V_{L2}.

In one embodiment, the binding protein of the disclosure comprises:
A polypeptide (I) consisting of an amino acid sequence of SEQ ID NO: 1; and
A polypeptide (II) consisting of an amino acid sequence of SEQ ID NO: 70.

In one embodiment, the Fc domains of the binding protein of the disclosure comprises N297 residue (according to Kabat numbering) mutated to prevent said residue to be glycosylated. Preferably, said mutation is a N297S substitution. In a preferred embodiment, such mutation substantially abolishes CD16A binding of the binding protein of the disclosure.

In one embodiment, the binding protein of the disclosure comprises:
A polypeptide (I) consisting of an amino acid sequence of SEQ ID NO: 66;
A polypeptide (II) consisting of an amino acid sequence of SEQ ID NO: 71.

In another embodiment, the Fc domains of the binding protein of the disclosure that comprises L234A, L235E, G237A, A330S and/or P331S substitutions according to kabat numbering.

Accordingly, one binding protein of the disclosure comprises:
A polypeptide (I) consisting of an amino acid sequence of SEQ ID NO: 68;
A polypeptide (II) consisting of an amino acid sequence of SEQ ID NO: 72.

Provided is a pharmaceutical composition comprising a binding protein of the disclosure and a pharmaceutically acceptable carrier.

Also provided is an isolated nucleic acid sequence comprising a nucleotide sequence that encodes a binding protein of the disclosure or a polypeptide chain thereof.

Provided is an expression vector comprising a nucleic acid of the disclosure, said nucleic acid sequence comprising a nucleotide sequence that encodes a binding protein of the disclosure or a polypeptide chain thereof.

Provided is an isolated cell comprising a nucleic acid of the disclosure, said nucleic acid sequence comprising a nucleotide sequence that encodes a binding protein of the disclosure or a polypeptide chain thereof.

Provided is an isolated cell comprising an expression vector of the disclosure, said expression vector comprising a nucleic acid of the disclosure, said nucleic acid sequence comprising a nucleotide sequence that encodes a binding protein of the disclosure or a polypeptide chain thereof.

Provided is a binding protein of the disclosure for use as a medicament.

Also provided is a binding protein of the disclosure for use in the treatment of a disease involving or characterized by CD20-expressing cells; and also a method of treating a disease in a subject involving or characterized by CD20-expressing cells, wherein said method comprising administering to the subject a binding protein of the disclosure.

In one embodiment, the disease treated by the binding protein for use of the disclosure or the method of treating of the disclosure a hematological cancer, e.g. a hematological cancer characterized by malignant cells that express CD20.

In another embodiment, the disease treated by the binding protein for use of the disclosure or the method of treating of the disclosure is selected from the group consisting of B cell lymphoma, Hodgkin's or non Hodgkin's B cell lymphoma, precursor B cell lymphoblastic leukemia/lymphoma and mature B cell neoplasms, such as B cell chronic lymphocytic leukemia (CLL)/small lymphocytic lymphoma (SLL), B cell prolymphocytic leukemia, lymphoplasmacytic lymphoma, mantle cell lymphoma (MCL), follicular lymphoma (FL), cutaneous follicle center lymphoma, marginal zone B cell lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenstrom's macroglobulinemia, and anaplastic large-cell lymphoma (ALCL).

In a further embodiment, the disease (e.g. NHL, CLL, SLL) treated by the binding protein for use of the disclosure or the method of treating of the disclosure is characterized by cells (e.g. cancer cells) expressing low levels of CD20 at their surface, or low numbers of CD20-expressing cells.

In one embodiment, the multispecific protein is administered between 1 and 4 times per month, optionally once every 2 week, optionally every 3 weeks, optionally once every 4 weeks, optionally further wherein treatment is for a period of at least 3 months, 6 months or 12 months.

Provided is a method for making a binding protein of the disclosure, comprising a step of:
(a) culturing host cell(s) under conditions suitable for expressing a plurality of recombinant polypeptides, said plurality comprising (i) a polypeptide comprising an amino acid sequence of SEQ ID NO: 1, 66 68, 77, 91 or 92, and (ii) a polypeptide comprising an amino acid sequence of SEQ ID NO: 9, 67, 69, 70, 71, 72, 73, 75, 76, 78 or 79 and optionally (iii) a polypeptide comprising an amino acid sequence of SEQ ID NO: 17 or 74;
(b) optionally recovering the expressed recombinant polypeptides.

In one embodiment, the method for making a binding protein of the disclosure comprises a step of:
(a) culturing host cell(s) under conditions suitable for expressing a plurality of recombinant polypeptides, said plurality comprising (i) a polypeptide comprising an amino acid of SEQ ID NO: 1, and (ii) a polypeptide comprising an amino acid sequence of SEQ ID NO: 70;
(b) optionally recovering the expressed recombinant polypeptides.

These and additional advantageous aspects and features of the invention may be further described elsewhere herein.

### BRIEF DESCRIPTION OF THE FIGURES

FIG. 1 shows an exemplary multispecific protein in T5 format that binds to NKp46, CD16A and CD122 on an NK cell, and to CD20 on a tumor cell.

FIGS. 2A to 2K show different configurations of multispecific proteins that differ in the number of polypeptide chain, and in the configuration of the domains around an Fc domain dimer.

FIG. 3 shows % of pSTAT5 cells among CD4 T cells, Tregs, CD8 T cells, NK cells upon the concentration of recombinant interleukin-2, CD20-1-T5-NKCE4-v3, CD20-2-T5-NKCE4-v3, CD20-3-T5-NKCE4-v3, CD20-4-T5-NKCE4-v3. All the tested multispecific proteins resulted in an increase in potency in the ability to induce pSTAT5+ cells among the NK cells, compared to recombinant IL-2. At the same time, all the tested multispecific proteins resulted in decrease in potency in the ability to induce pSTAT5+ cells among the CD4 T cells and Treg cells, compared to recombinant IL-2 The multispecific proteins thus permitted a preferential activation of NK cells over Treg cells, CD4 T cells and CD8 T cells.

FIG. 4 shows the binding potency of several CD20-1-T5-NKCE4, CD20-2-T5-NKCE4, CD20-3-T5-NKCE4, CD20-4-T5-NKCE4 toward a RAJI cell line. The media fluorescence intensity measured is shown on the y-axis and concentration of test protein is shown on the x-axis. The CD20-2-T5-NKCE4 protein showed higher efficacy in binding to CD20+ Raji cells as compared to the other molecules.

FIG. 5 is a biacore sensogram demonstrating the ability of CD20-2-T5A-NKCE4-v2A to selectively binds to CD122 receptors. CM5 chip was used comprising immobilized anti-His antibody (210322CCe, 1002 RU). HuCD25-His (cycle 2), HuCD122-His (cycle 1) or HuCD132-His (cycle 4) was injected at the beginning of each cycle to be capture on the chip. CD20-2-T5A-NKCE4-v2A (1 μM) was then injected during 120 s at 10 μL/min. The interaction between CD20-2-T5A-NKCE4-v2A and HuCD25-His, HuCD122-His or HuCD132-His was studied with a dissociation time of 600 s.

FIG. 6 shows % of cytotoxicity induced by NK cells on the y-axis and concentration of test protein on the x-axis in the presence of each of the several NKCE proteins. All CD20-T5-NKCE4-v3 proteins, whatever their CD20 ABD, were highly potent in ability to mediate NK cell cytotoxicity toward tumor target cells. The IC-T5-NKCE4-v3 control molecule that does not bind to CD20 on RAJI tumor cells did not induce cytoxicity. The CD20-2-T5-NKCE4-v3 induced a significantly better induction of NK cell cytotoxicity on RAJI tumor cells that other molecules.

FIG. 7 shows the tumor volume in mice after administration of 0.4 μg, 2 μg or 10 μg of CD20-2-T13-NKCE4-v2a or CD20-1-T5-NKCE4. Tumor was engrafted on Day 0 and a single dose of 0.4 μg, 2 μg or 10 μg of CD20-2-T13-NKCE4-v2a or CD20-1-T5-NKCE4 was administered on Day 9. Each dot on the figure represents tumor volume in an individual animal. A dose of 10 μg of CD20-2-T13-NKCE4-v2A or CD20-2-T5-NKCE4 showed strong efficacy as a single injection compared to vehicle alone.

FIGS. 8A and B show % of cytotoxicity induced by NK cells on the y-axis and concentration of test protein on the x-axis. All NKCE4 proteins whatever their format, were highly potent in ability to mediate NK cell cytotoxicity toward tumor target cells.

FIG. 9 shows the proliferation of a NK cell line (RLU) incubated with CD20-2-T13-NKCE4-v2A, and CD20-1-T5-NKCE4. The data showed that CD20-2-T13-NKCE4-v2A was more potent than the CD20-1-T5-NKCE4 molecule to induce NK cell proliferation.

FIG. 10A shows the sera concentration of CD20-NKCE4 over time after injection (at day 0) in non human primates (n=4 per tested condition). FIG. 10B shows several pharmacokinetic parameters of CD20-NKCE4 proteins (EC50 of STAT5 phosphorylation, cytotoxicity and proliferation of NK cells, as well as maximal concentration of CD20-NKCE4 proteins in non-human primate serum (Cmax) and serum concentration 22 days after injection.

FIGS. 11A and 11B show the percentage of B cells over time and the number of B cells counted after incubation of several CD20-NKCE4 proteins in human PBMCs (CD20-2-T13-NKCE4-v2A or CD20-2-F13-NKCE3) or controls (IC-T13-NKCE4-v2A, huIL2v2A-His-BirA or no antibody) for 24 h. In contrast to control molecules (IC-T13-NKCE4-v2A, huIL2v2A-His-BirA), the CD20-2-T13-NKCE4-v2A and CD20-2-F13-NKCE3 were each able to deplete CD20+ B cells.

FIGS. 12A and 12B show the percentage of T cells over time and the number of T cells counted after incubation of several CD20-NKCE4 proteins in human PBMCs (CD20-2-T13-NKCE4-v2A or CD20-2-F13-NKCE3) or controls (IC-T13-NKCE4-v2A, huIL2v2A-His-BirA or no antibody) for 24 h. The data show that CD20-2-T13-NKCE4-v2A and CD20-2-F13-NKCE3 do not deplete non CD20+ T cells.

FIGS. 13A and 13B show the percentage of NK cells over time and the number of NK cells counted after incubation of several CD20-NKCE4 proteins in non human primates (CD20-2-T13-NKCE4-v2A or CD20-2-F13-NKCE3) or controls (IC-T13-NKCE4-v2A, huIL2v2A-His-BirA or no antibody) for 24 h. The data show no decrease of NK cells induced by CD20-2-T13-NKCE4-v2A suggesting no fratricidal killing among NK cells.

FIG. 14 shows the concentration of several cytokines (IFN-γ, IL-6, TNF-α, IL-10, IL-8, MIP-1β, MCP-1, IL-1β) over time after injection of several CD20-NKCE4 in non-human primates.

FIG. 15 shows the evolution of the number of circulating B cell over time after injection of different CD20-NKCE4 proteins in non-human primates.

FIGS. 16A, 16B and 16C show the evolution overtime of B, NK and T cell populations in non-human primates upon administration of CD20-2-T13-NKCE4-v2A at day 0, 7 and 14.

### DETAILED DESCRIPTION OF THE INVENTION

### Definitions

As used in the specification, “a” or “an” may mean one or more. As used in the claim(s), when used in conjunction with the word “comprising”, the words “a” or “an” may mean one or more than one.

Where “comprising” is used, this can optionally be replaced by “consisting essentially of”, or optionally by “consisting of”.

As used herein, the term “antigen binding domain” or “ABD” refers to a domain comprising a three-dimensional structure capable of immunospecifically binding to an epitope. Thus, in one embodiment, said domain can comprise a hypervariable region, optionally a V_{H} and/or V_{L} domain of an antibody chain, optionally at least a V_{H} domain. In another embodiment, the binding domain may comprise at least one complementarity determining region (CDR) of an antibody chain. In another embodiment, the binding domain may comprise a polypeptide domain from a non-immunoglobulin scaffold.

The term “antibody” herein is used in the broadest sense and specifically includes full-length monoclonal antibodies, polyclonal antibodies, multispecific antibodies (e.g., bispecific antibodies), and antibody fragments and derivatives, so long as they exhibit the desired biological activity. Various techniques relevant to the production of antibodies are provided in, e.g., Harlow, et al., ANTIBODIES: A LABORATORY MANUAL, Cold Spring Harbor Laboratory Press, Cold Spring Harbor, N.Y., (1988). An “antibody fragment” comprises a portion of a full-length antibody, e.g. antigen-binding or variable regions thereof. Examples of antibody fragments include Fab, Fab′, F(ab)₂, F(ab′)₂, F(ab)₃, Fv (typically the V_{L} and V_{H} domains of a single arm of an antibody), single-chain Fv (scFv), dsFv, Fd fragments (typically the V_{H} and CH1 domain), and dAb (typically a V_{H} domain) fragments; V_{H}, V_{L}, VhH, and V-NAR domains; minibodies, diabodies, triabodies, tetrabodies, and kappa bodies (see, e.g., III et al., Protein Eng 1997; 10: 949-57); camel IgG; IgNAR; and multispecific antibody fragments formed from antibody fragments, and one or more isolated CDRs or a functional paratope, where isolated CDRs or antigen-binding residues or polypeptides can be associated or linked together so as to form a functional antibody fragment. Various types of antibody fragments have been described or reviewed in, e.g., Holliger and Hudson, Nat Biotechnol 2005; 23, 1126-1136; WO2005040219, and published U.S. Patent Applications 20050238646 and 20020161201.

The term “hypervariable region” when used herein refers to the amino acid residues of an antibody that are responsible for antigen binding. The hypervariable region generally comprises amino acid residues from a “complementarity-determining region” or “CDR” (e.g. residues 24-34 (L1), 50-56 (L2) and 89-97 (L3) in the light-chain variable domain and 31-35 (H1), 50-65 (H2) and 95-102 (H3) in the heavy-chain variable domain; Kabat et al. 1991) and/or those residues from a “hypervariable loop” (e.g. residues 26-32 (L1), 50-52 (L2) and 91-96 (L3) in the light-chain variable domain and 26-32 (H1), 53-55 (H2) and 96-101 (H3) in the heavy-chain variable domain; Chothia and Lesk, J. Mol. Biol 1987; 196:901-917). Typically, the numbering of amino acid residues in this region is performed by the method described in Kabat et al., supra. Phrases such as “Kabat position”, “variable domain residue numbering as in Kabat” and “according to Kabat” herein refer to this numbering system for heavy chain variable domains or light chain variable domains. Using the Kabat numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of CDR H2 and inserted residues (e.g. residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

By “framework” or “FR” residues as used herein is meant the region of an antibody variable domain exclusive of those regions defined as CDRs. Each antibody variable domain framework can be further subdivided into the contiguous regions separated by the CDRs (FR1, FR2, FR3 and FR4).

By “constant region” as defined herein is meant an antibody-derived constant region that is encoded by one of the light or heavy chain immunoglobulin constant region genes.

By “constant light chain” or “light chain constant region” or “CL” as used herein is meant the region of an antibody encoded by the kappa (Cκ) or lambda (Cλ) light chains. The constant light chain typically comprises a single domain, and as defined herein refers to positions 108-214 of Cκ, or Cλ, wherein numbering is according to the EU index (Kabat et al., 1991, Sequences of Proteins of Immunological Interest, 5th Ed., United States Public Health Service, National Institutes of Health, Bethesda).

By “constant heavy chain” or “heavy chain constant region” as used herein is meant the region of an antibody encoded by the mu, delta, gamma, alpha, or epsilon genes to define the antibody's isotype as IgM, IgD, IgG, IgA, or IgE, respectively. For full length IgG antibodies, the constant heavy chain, as defined herein, refers to the N-terminus of the CH1 domain to the C-terminus of the CH3 domain, thus comprising positions 118-447, wherein numbering is according to the EU index.

As used herein, the terms “C_{H}1 domain”, or “C_{H1} domain”, or “constant domain 1”, can be used interchangeably and refer to the corresponding heavy chain immunoglobulin constant domain 1.

As used herein, the term “CH2 domain”, or “CH₂ domain”, or “constant domain 2” can be used interchangeably and refer to the corresponding heavy chain immunoglobulin constant domain 2.

As used herein, the term “CH3 domain”, or “C_{H}3 domain”, or “constant domain 3” can be used interchangeably and refer to the corresponding heavy chain immunoglobulin constant domain 3.

As used herein, the term “CH2-CH3”, as in (CH2-CH3)_{A} and (CH2-CH3)_{B}, thus refers to a polypeptide sequence comprising an immunoglobulin heavy chain constant domain 2 (CH2) and an immunoglobulin heavy chain constant domain 3 (CH3).

As used herein, the terms “pair C (CH1/CL)”, or “paired C (C_{H}1/C_{L})” “refers to one constant heavy chain domain 1 and one constant light chain domain (e.g. a kappa (κ or κ) or lamba (λ) class of immunoglobulin light chains) bound to one another by covalent or non-covalent bonds, preferably non-covalent bonds; thus forming a heterodimer. Unless specified otherwise, when the constant chain domains forming the pair are not present on a same polypeptide chain, this term may thus encompass all possible combinations. Preferably, the corresponding C_{H}1 and C_{L} domains will thus be selected as complementary to each other, such that they form a stable pair C (CH1/CL).

Advantageously, when the binding protein comprises a plurality of paired C domains, such as one “pair C₁ (C_{H}1/C_{L})” and one “pair C₂ (C_{H}1/C_{L})”, each CH1 and CL domain forming the pairs will be selected so that they are formed between complementary CH1 and CL domains. Examples of complementary C_{H}1 and C_{L} domains have been previously described in the international patent applications WO2006/064136 or WO2012/089814 or WO2015197593A1.

Unless instructed otherwise, the terms “pair C₁ (C_{H}1/C_{L})” or “pair C₂ (C_{H}1/C_{L})” may refer to distinct constant pair domains (C₁ and C₂) formed by identical or distinct constant heavy 1 domains (C_{H}1) and identical or distinct constant light chain domains (C_{L}). Preferably, the terms “pair C₁ (C_{H}1/C_{L})” or “pair C2 (C_{H}1/CL)” may refer to distinct constant pair domains (C₁ and C₂) formed by identical constant heavy 1 domains (C_{H}1) and identical constant light chain domains (C_{L}).

By “Fab” or “Fab region” as used herein is meant a unit that comprises the V_{H}, CH1, V_{L}, and CL immunoglobulin domains. The term Fab includes a unit that comprises a V_{H}-CH1 moiety that associates with a V_{L}—CL moiety, as well as crossover Fab structures in which there is crossing over or interchange between light- and heavy-chain domains. For example a Fab may have a V_{H}-CL unit that associates with a V_{L}—CH1 unit. Fab may refer to this region in isolation, or this region in the context of a protein, multispecific protein or ABD, or any other embodiments as outlined herein.

By “single-chain Fv” or “scFv” as used herein are meant antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the scFv to form the desired structure for antigen binding. Methods for producing scFvs are well known in the art. For a review of methods for producing scFvs see Pluckthun in The Pharmacology of Monoclonal Antibodies, vol. 113, Rosenburg and Moore eds. Springer-Verlag, New York, pp. 269-315 (1994).

By “Fv” or “Fv fragment” or “Fv region” as used herein is meant a polypeptide that comprises the V_{L} and V_{H} domains of a single antibody.

By “Fc” or “Fc region”, as used herein is meant the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains Cy2 (CH2) and Cy3 (CH3) and optionally the hinge between Cy1 and Cy2. Although the boundaries of the Fc region may vary, the human IgG heavy chain Fc region is usually defined to comprise residues C226, P230 or A231 to its carboxyl-terminus, wherein the numbering is according to the EU index. Fc may refer to this region in isolation, or this region in the context of an Fc polypeptide, as described below. By “Fc polypeptide” or “Fc-derived polypeptide” as used herein is meant a polypeptide that comprises all or part of an Fc region. Fc polypeptides herein include but are not limited to antibodies, Fc fusions and Fc fragments. Also, Fc regions according to the invention include variants containing at least one modification that alters (enhances or diminishes) an Fc associated effector function. Also, Fc regions according to the invention include chimeric Fc regions comprising different portions or domains of different Fc regions, e.g., derived from antibodies of different isotype or species.

By “variable region” as used herein is meant the region of an antibody that comprises one or more Ig domains substantially encoded by any of the V_{L} (including Vκ (Vκ) and Vλ) and/or V_{H} genes that make up the light chain (including κ and λ) and heavy chain immunoglobulin genetic loci respectively. A light or heavy chain variable region (V_{L} or V_{H}) consists of a “framework” or “FR” region interrupted by three hypervariable regions referred to as “complementarity determining regions” or “CDRs”. The extent of the framework region and CDRs have been precisely defined, for example as in Kabat (see “Sequences of Proteins of Immunological Interest,” E. Kabat et al., U.S. Department of Health and Human Services, (1983)), and as in Chothia. The framework regions of an antibody, that is the combined framework regions of the constituent light and heavy chains, serves to position and align the CDRs, which are primarily responsible for binding to an antigen.

As used herein, the term “domain” may be any region of a protein, generally defined on the basis of sequence homologies or identities, which is related to a specific structural or functional entity. Accordingly, the term “region”, as used in the context of the present disclosure, is broader in that it may comprise additional regions beyond the corresponding domain.

As used herein, the terms “linker region”, “linker peptide” or “linker polypeptide” or “amino acid linker” or “linker” refer to any amino acid sequence suitable for covalently linking two polypeptide domains, such as two antigen-binding domains together and/or a Fc region to one or more variable regions, such as one or more antigen-binding domains. Although the term is not limited to a particular size or polypeptide length, such amino acid linkers are generally less than 50 amino acids in length, preferably less than 30 amino acids in length, for instance 20 or less than 20 amino acids in length, for instance 15 or less than 15 amino acids in length. Such amino acid linkers may optionally comprise all or part of an immunoglobulin polypeptide chain, such as all or part of a hinge region of an immunoglobulin. Alternatively, the amino acid linker may comprise a polypeptide sequence that is not derived from a hinge region of an immunoglobulin, or even that is not derived from an immunoglobulin heavy or light polypeptide chain.

As used herein, an immunoglobulin hinge region, or a fragment thereof, may thus be considered as a particular type of linker, which is derived from an immunoglobulin polypeptide chain.

As used herein, the term “hinge region” or “hinge” refers to a generally flexible region and born by the corresponding heavy chain polypeptides, and which separates the Fc and Fab portions of certain isotypes of immunoglobulins, more particularly of the IgG, IgA or IgD isotypes. Such hinge regions are known in the Art to depend upon the isotype of immunoglobulin which is considered. For native IgG, IgA and IgD isotypes, the hinge region thus separates the C_{H}1 domain and the C_{H}2 domain and is generally cleaved upon papain digestion. On the other hand, the region corresponding to the hinge in IgM and IgE heavy chains is generally formed by an additional constant domain with lower flexibility. Additionally, the hinge region may comprise one or more cysteines involved in interchain disulfide bonds. The hinge region may also comprise one or more binding sites to a Fcγ receptor, in addition to FcγR binding sites born by the C_{H}2 domain, when applicable. Additionally, the hinge region may comprise one or more post-translational modification, such as one or more glycosylated residues depending on the isotype which is considered. Thus, it will be readily understood that the reference to the term “hinge” throughout the specification is not limited to a particular set of hinge sequences or to a specific location on the structure. Unless instructed otherwise, the hinge regions which are still particularly considered comprise all or part of a hinge from an immunoglobulin belonging to one isotype selected from: the IgG isotype, the IgA isotype and the IgD isotype; in particular the IgG isotype.

The term “specifically binds to” means that an antibody or polypeptide can bind preferably in a competitive binding assay to the binding partner, e.g. NKp46, as assessed using either recombinant forms of the proteins, epitopes therein, or native proteins present on the surface of isolated target cells. Competitive binding assays and other methods for determining specific binding are further described below and are well known in the art.

When an antibody or polypeptide is said to “compete with” a particular multispecific protein or a particular monoclonal antibody (e.g. NKp46-1, -2, -4, -6 or -9 in the context of an anti-NKp46 mono-specific antibody or a multi-specific protein), it means that the antibody or polypeptide competes with the particular multispecific protein or monoclonal antibody in a binding assay using either recombinant target (e.g. NKp46) molecules or surface expressed target (e.g. NKp46) molecules. For example, if a test antibody reduces the binding of NKp46-1, -2, -4, -6 or -9 to a NKp46 polypeptide or NKp46-expressing cell in a binding assay, the antibody is said to “compete” respectively with NKp46-1, -2, -4, -6 or -9.

The term “affinity”, as used herein, means the strength of the binding of an antibody or protein to an epitope. The affinity of an antibody is given by the dissociation constant K_{D}, defined as [Ab]×[Ag]/[Ab−Ag], where [Ab−Ag] is the molar concentration of the antibody-antigen complex, [Ab] is the molar concentration of the unbound antibody and [Ag] is the molar concentration of the unbound antigen. The affinity constant K_{A} is defined by 1/K_{D}. Preferred methods for determining the affinity of proteins can be found in Harlow, et al., Antibodies: A Laboratory Manual, Cold Spring Harbor Laboratory Press, Cold Spring Harbor, N.Y., 1988), Coligan et al., eds., Current Protocols in Immunology, Greene Publishing Assoc. and Wiley Interscience, N.Y., (1992, 1993), and Muller, Meth. Enzymol. 92:589-601 (1983), which references are entirely incorporated herein by reference. One preferred and standard method well known in the art for determining the affinity of proteins is the use of surface plasmon resonance (SPR) screening (such as by analysis with a BIAcore™ SPR analytical device).

Within the context of this invention a “determinant” designates a site of interaction or binding on a polypeptide.

The term “epitope” refers to an antigenic determinant, and is the area or region on an antigen to which an antibody or protein binds. A protein epitope may comprise amino acid residues directly involved in the binding as well as amino acid residues which are effectively blocked by the specific antigen binding antibody or peptide, i.e., amino acid residues within the “footprint” of the antibody. It is the simplest form or smallest structural area on a complex antigen molecule that can combine with e.g., an antibody or a receptor. Epitopes can be linear or conformational/structural. The term “linear epitope” is defined as an epitope composed of amino acid residues that are contiguous on the linear sequence of amino acids (primary structure). The term “conformational or structural epitope” is defined as an epitope composed of amino acid residues that are not all contiguous and thus represent separated parts of the linear sequence of amino acids that are brought into proximity to one another by folding of the molecule (secondary, tertiary and/or quaternary structures). A conformational epitope is dependent on the 3-dimensional structure. The term ‘conformational’ is therefore often used interchangeably with ‘structural’. Epitopes may be identified by different methods known in the art including but not limited to alanine scanning, phage display, X-ray crystallography, array-based oligo-peptide scanning or pepscan analysis, site-directed mutagenesis, high throughput mutagenesis mapping, H/D-Ex Mass Spectroscopy, homology modeling, docking, hydrogen-deuterium exchange, among others. (See e.g., Tong et al., Methods and Protocols for prediction of immunogenic epitopes”, Briefings in Bioinformatics 8(2):96-108; Gershoni, Jonathan M; Roitburd-Berman, Anna; Siman-Tov, Dror D; Tarnovitski Freund, Natalia; Weiss, Yael (2007). “Epitope Mapping”. BioDrugs 21 (3): 145-56; and Flanagan, Nina (May 15, 2011); “Mapping Epitopes with H/D-Ex Mass Spec: ExSAR Expands Repertoire of Technology Platform Beyond Protein Characterization”, Genetic Engineering & Biotechnology News 31 (10).

“Valent” or “valency” denotes the presence of a determined number of antigen-binding moieties in the antigen-binding protein. A natural IgG has two antigen-binding moieties and is bivalent. A molecule having one binding moiety for a particular antigen is monovalent for that antigen.

By “amino acid modification” herein is meant an amino acid substitution, insertion, and/or deletion in a polypeptide sequence. An example of amino acid modification herein is a substitution. By “amino acid modification” herein is meant an amino acid substitution, insertion, and/or deletion in a polypeptide sequence. By “amino acid substitution” or “substitution” herein is meant the replacement of an amino acid at a given position in a protein sequence with another amino acid. For example, the substitution Y50W refers to a variant of a parent polypeptide, in which the tyrosine at position 50 is replaced with tryptophan. Amino acid substitutions are indicated by listing the residue present in wild-type protein/position of residue/residue present in mutant protein. A “variant” of a polypeptide refers to a polypeptide having an amino acid sequence that is substantially identical to a reference polypeptide, typically a native or “parent” polypeptide. The polypeptide variant may possess one or more amino acid substitutions, deletions, and/or insertions at certain positions within the native amino acid sequence.

“Conservative” amino acid substitutions are those in which an amino acid residue is replaced with an amino acid residue having a side chain with similar physicochemical properties. Families of amino acid residues having similar side chains are known in the art, and include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine).

The term “identity” or “identical”, when used in a relationship between the sequences of two or more polypeptides, refers to the degree of sequence relatedness between polypeptides, as determined by the number of matches between strings of two or more amino acid residues. “Identity” measures the percent of identical matches between the smaller of two or more sequences with gap alignments (if any) addressed by a particular mathematical model or computer program (i.e., “algorithms”). Identity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math. 48, 1073 (1988).

Preferred methods for determining identity are designed to give the largest match between the sequences tested. Methods of determining identity are described in publicly available computer programs. Preferred computer program methods for determining identity between two sequences include the GCG program package, including GAP (Devereux et al., Nucl. Acid. Res. 12, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

An “isolated” molecule is a molecule that is the predominant species in the composition wherein it is found with respect to the class of molecules to which it belongs (i.e., it makes up at least about 50% of the type of molecule in the composition and typically will make up at least about 70%, at least about 80%, at least about 85%, at least about 90%, at least about 95%, or more of the species of molecule, e.g., peptide, in the composition). Commonly, a composition of a polypeptide will exhibit 98%, 98%, or 99% homogeneity for polypeptides in the context of all present peptide species in the composition or at least with respect to substantially active peptide species in the context of proposed use.

In the context herein, “treatment” or “treating” refers to preventing, alleviating, managing, curing or reducing one or more symptoms or clinically relevant manifestations of a disease or disorder, unless contradicted by context. For example, “treatment” of a patient in whom no symptoms or clinically relevant manifestations of a disease or disorder have been identified is preventive or prophylactic therapy, whereas “treatment” of a patient in whom symptoms or clinically relevant manifestations of a disease or disorder have been identified generally does not constitute preventive or prophylactic therapy.

As used herein, the phrase “NK cells” refers to a sub-population of lymphocytes that is involved in non-conventional immunity. NK cells can be identified by virtue of certain characteristics and biological properties, such as the expression of specific surface antigens including CD56 and/or NKp46 for human NK cells, the absence of the alpha/beta or gamma/delta TCR complex on the cell surface, the ability to bind to and kill cells that fail to express “self” MHC/HLA antigens by the activation of specific cytolytic machinery, the ability to kill tumor cells or other diseased cells that express a ligand for NK activating receptors, and the ability to release protein molecules called cytokines that stimulate or inhibit the immune response. Any of these characteristics and activities can be used to identify NK cells, using methods well known in the art. Any subpopulation of NK cells will also be encompassed by the term NK cells. Within the context herein “active” NK cells designate biologically active NK cells, including NK cells having the capacity of lysing target cells or enhancing the immune function of other cells. NK cells can be obtained by various techniques known in the art, such as isolation from blood samples, cytapheresis, tissue or cell collections, etc. Useful protocols for assays involving NK cells can be found in Natural Killer Cells Protocols (edited by Campbell K S and Colonna M). Humana Press. pp. 219-238 (2000).

As used herein, an agent that has “agonist” activity at NKp46 is an agent that can cause or increase “NKp46 signaling”. “NKp46 signaling” refers to an ability of an NKp46 polypeptide to activate or transduce an intracellular signaling pathway. Changes in NKp46 signaling activity can be measured, for example, by assays designed to measure changes in NKp46 signaling pathways, e.g. by monitoring phosphorylation of signal transduction components, assays to measure the association of certain signal transduction components with other proteins or intracellular structures, or in the biochemical activity of components such as kinases, or assays designed to measure expression of reporter genes under control of NKp46-sensitive promoters and enhancers, or indirectly by a downstream effect mediated by the NKp46 polypeptide (e.g. activation of specific cytolytic machinery in NK cells). Reporter genes can be naturally occurring genes (e.g. monitoring cytokine production) or they can be genes artificially introduced into a cell. Other genes can be placed under the control of such regulatory elements and thus serve to report the level of NKp46 signaling.

“NKp46” refers to a protein or polypeptide encoded by the Ncr1 gene or by a cDNA prepared from such a gene. Any naturally occurring isoform, allele, ortholog or variant is encompassed by the term NKp46 polypeptide (e.g., an NKp46 polypeptide 90%, 95%, 98% or 99% identical to SEQ ID NO 1, or a contiguous sequence of at least 20, 30, 50, 100 or 200 amino acid residues thereof). The 304 amino acid residue sequence of human NKp46 (isoform a) is shown below:

TABLE 1

| | |
|---|---|
| SEQ | MSSTLPALLC VGLCLSQRIS AQQQTLPKPF IWAEPHFMVP KEKQVTICCQ |
| ID NO: | GNYGAVEYQL HFEGSLFAVD RPKPPERINK VKFYIPDMNS RMAGQYSCIY |
| 88 | RVGELWSEPS NLLDLVVTEM YDTPTLSVHP GPEVISGEKV TFYCRLDTAT |
| | SMFLLLKEGR SSHVQRGYGK VQAEFPLGPV TTAHRGTYRC FGSYNNHAWS |
| | FPSEPVKLLV TGDIENTSLA PEDPTFPADT WGTYLLTTET GLQKDHALWD |
| | HTAQNLLRMG LAFLVLVALV WFLVEDWLSR KRTRERASRA STWEGRRRLN |
| | TQTL |

SEQ ID NO: 88 corresponds to NCBI accession number NP_004820, the disclosure of which is incorporated herein by reference. The human NKp46 mRNA sequence is described in NCBI accession number NM_004829, the disclosure of which is incorporated herein by reference.

As used herein, the term “subject” or “individual” or “patient” are used interchangeably and may encompass a human or a non-human mammal, rodent or non-rodent. The term includes, but is not limited to, mammals, e.g., humans including man, woman and child, other primates (monkey), pigs, rodents such as mice and rats, rabbits, guinea pigs, hamsters, cows, horses, cats, dogs, sheep and goats.

### Producing Polypeptides

The proteins described herein can be conveniently configured and produced using well known immunoglobulin-derived domains, notably heavy and light chain variable domains, hinge regions, CH1, CL, CH2 and CH3 constant domains, and wild-type or variant cytokine polypeptides. Domains placed on a common polypeptide chain can be fused to one another either directly or connected via linkers, depending on the particular domains concerned. The immunoglobulin-derived domains will preferably be humanized or of human origin, thereby providing decreased risk of immunogenicity when administered to humans. As shown herein, advantageous protein formats are described that use minimal non-immunoglobulin linking amino acid sequences (e.g. not more than 4 or 5 domain linkers, in some cases as few as 1 or 2 domain linkers, and use of domains linkers of short length), thereby further reducing risk of immunogenicity.

Immunoglobulin variable domains are commonly derived from antibodies (immunoglobulin chains), for example in the form of associated V_{L} and V_{H} domains found on two polypeptide chains, or a single chain antigen binding domain such as an scFv, a V_{H} domain, a V_{L} domain, a dAb, a V-NAR domain or a V_{H}H domain. In certain advantageous proteins formats disclosed herein that directly enable the use of a wide range of variable regions from Fab or scFv without substantial further requirements for pairing and/or folding, the antigen binding domain (e.g., ABD₁ and ABD₂) can also be readily derived from antibodies as a Fab or scFv.

The term “antigen-binding protein” can be used to refer to an immunoglobulin derivative with antigen binding properties. The binding protein comprises an immunologically functional immunoglobulin portion capable of binding to a target antigen. The immunologically functional immunoglobulin portion may comprise immunoglobulins, or portions thereof, fusion peptides derived from immunoglobulin portions or conjugates combining immunoglobulin portions that form an antigen binding site. Each antigen binding moiety comprises at least the necessarily one, two or three CDRs of the immunoglobulin heavy and/or light chains from which the antigen binding moiety was derived. In some aspects, an antigen-binding protein can consist of a single polypeptide chain (a monomer). In other embodiments the antigen-binding protein comprises at least two polypeptide chains, e.g. a multimeric protein, optionally specified as being a dimeric protein trimeric protein. As further exemplified herein, an antigen binding domain can conveniently comprise a VH and a VL (a VH/VL pair). In some embodiments, the VH/VL pair can be integrated in a Fab structure further comprising a CH1 and CL domain (a CH1/CL pair). A VH/VL pair refers to one VH and one VL domain that associate with one another to form an antigen binding domain. A CH1/CL pair refers to one CH1 and one CL domain bound to one another by covalent or non-covalent bonds, preferably non-covalent bonds, thus forming a heterodimer (e.g., within a protein such as a heterotrimer that can comprise one or more further polypeptide chains).

In one embodiment, a binding protein comprises:
(i) a first antigen-binding domain (ABD) comprising a variable region which binds specifically to a human CD20 polypeptide, (ii) a second antigen-binding domain (ABD) comprising a variable region which binds specifically to a human NKp46 polypeptide, (iii) all or part of an immunoglobulin Fc region or variant thereof which binds to a human Fc-γ receptor (CD16), and cytokine moiety.

### Protein Formats

Multimeric, multispecific proteins such as heterodimers and heterotrimers can be produced according to a variety of formats. Different domains onto different polypeptide chain that associate to form a multimeric protein. Accordingly, a wide range of protein formats can be constructed around Fc domain dimers that are capable of binding to human FcRn polypeptide (neonatal Fc receptor), with or without additionally binding to CD16 or CD16A, depending on whether or not such CD16 binding ABD is desired to be present. As shown herein, greatest potentiation of NK cell cytotoxicity can be obtained through use of Fc moieties that have substantial binding to the activating human CD16 receptor (CD16A) binding; such CD16 binding can be obtained through the use of suitable CH2 and/or CH3 domains, as further described herein. In one embodiment, an Fc moiety is derived from a human IgG1 isotype constant region. Use of modified CH3 domains also contributes to the possibility of use a wide range of heteromultimeric protein structures. Accordingly, a protein comprises a first and a second polypeptide chain each comprising a variable domain fused to a human Fc domain monomer (i.e. a CH2-CH3 unit), optionally a Fc domain monomer comprising a CH3 domain capable of undergoing preferential CH3-CH3 hetero-dimerization, wherein the first and second chain associate via CH3-CH3 dimerization and the protein consequently comprises a Fc domain dimer. The variable domains of each chain can be part of the same or different antigen binding domains.

Multispecific proteins can thus be conveniently constructed using VH and VL pairs arranged as scFv or Fab structures, together with CH1 domains, CL domain, Fc domains and cytokines, and domain linkers. Preferably, the proteins will use minimal non-natural sequences, e.g. minimal use of non-Ig linkers, optionally no more than 5, 4, 3, 2 or 1 domain linker(s) that is not an antibody-derived sequence, optionally wherein domain linker(s) are no more than 15, 10 or 5 amino acid residues in length. In one embodiment, the protein comprises a CD16 ABD embodied as a Fc domain dimer.

In some embodiment, the multispecific proteins (e.g. dimers, trimers) may comprise a domain arrangement of any of the following in which domains can be placed on any of the 2 or 3 polypeptide chains, wherein the NKp46 ABD is interposed between the Fc domain and the cytokine moiety (e.g. the protein has a terminal or distal cytokine receptor ABD at the C-terminal end and a terminal or distal CD20 ABD at the topological N-terminal end), wherein the NKp46 ABD is connected to one of the polypeptide chains of the Fc domain dimer via a hinge polypeptide or a flexible linker, and wherein the ABD that binds the cytokine receptor is connected to NKp46 ABD (e.g. to one of the polypeptide chains thereof when the NKp46 ABD is contained on two chains) via a flexible linker (e.g. a linker comprising G and S residues):

(Anti-CD20 ABD)-(Fc domain dimer)-(NKp46 ABD)-(cytokine moiety).

The cytokine moiety can be an IL2 polypeptide or variant thereof. The Fc domain dimer can be specified to be a Fc domain dimer that binds human FcRn and/or Fcγ receptors. In one embodiment, one or both of the CD20 ABD and NKp46 ABD is formed from two variable regions present, wherein the variable regions that associate to form a particular ABD can be on the same polypeptide chain or on different polypeptide chains. In another embodiment, one or both of CD20 ABD and NKp46 ABD comprises a tandem variable region (scFv) and the other comprises a Fab structure. In another embodiment, both of the antigen of interest and NKp46 ABD comprises a Fab structure. In another embodiment the CD20 ABD comprises a Fab structure and the NKp46 ABD comprises an scFv structure.

In one embodiment, the binding protein of the disclosure is heterotrimeric and comprises three polypeptide chains (I), (II) and (III) that form two ABDs, as defined above:

V_{1A}-C_{1A}-Hinge₁-(Fc domain)_{A} (I)

V_{1B}-C_{1B}-Hinge₂-(Fc domain)_{B}-L₁-V_{2A}-C_{2A} (II)

V_{2B}-C_{2B}-Hinge₃-L₂-IL-2 (III)
wherein:
V_{1A} and V_{1B} form a binding pair V₁ (V_{H1}/V_{L1});
V_{2A} and V_{2B} form a binding pair V₂ (V_{H2}/V_{L2});
C_{1A} and C_{1B} form a pair C₁ (CH1/C_{L}) and C_{2A} and C_{2B} form a pair C₂ (CH1/C_{L}) wherein CH1 is an immunoglobulin heavy chain constant domain 1 and C_{L} is an immunoglobulin light chain constant domain;
Hinge₁, Hinge₂ and Hinge₃ are identical or different and correspond to all or part of an immunoglobulin hinge region;
(Fc domain)_{A} and (Fc domain)_{B} are identical or different, and comprise a CH2-CH3 domain;
L₁ and L₂ are an amino acid linker, wherein L₁ and L₂ can be different or the same;
IL-2 is a variant human interleukin-2 polypeptide or portion thereof that binds to CD122 present on NK cells. In one embodiment, binding pair V₁ binds CD20 and binding pair V₂ binds NKp46.

Each of V_{1A}, V_{1B}, V_{2A}, V_{2B} are an immunoglobulin VH or VL domain, wherein one of V1A and V_{1B} is a VH and the other is a VL, and wherein one of V2A and V_{2B} is a VH and the other is a VL.

In some embodiments, the binding protein of the disclosure has a residue N297 of the Fc domain or variant thereof according to Kabat numbering that comprises a N-linked glycosylation. In some embodiments, the binding protein of the disclosure comprises an Fc domain that binds to human CD16A polypeptide.

According to some embodiments, V1A is VL1 and V1B is VH1.

According to some embodiments, V2A is VH2 and V2B is VL2.

According to some embodiments, C1A is CK and C1B is CH1.

According to some embodiments, C2A is CK and C2B is CH1.

In some embodiments, VH1 comprises a CDR1, CDR2 and CDR3 corresponding to the amino acid sequences of SEQ ID NO: 29 (HCDR1), SEQ ID NO: 32 (HCDR2), SEQ ID NO: 35 (HCDR3); VL1 comprises a CDR1, CDR2 and CDR3 corresponding to the amino acid sequences of SEQ ID NO: 38 (LCDR1), SEQ ID NO: 41 (LCDR2), SEQ ID NO: 44 (LCDR3); VH2 comprises a CDR1, CDR2 and CDR3 corresponding to the amino acid sequences of SEQ ID NO: 47 (HCDR1), SEQ ID NO: 50 (HCDR2), SEQ ID NO: 53 (HCDR3), and VL2 comprises a CDR1, CDR2 and CDR3 corresponding to the amino acid sequences of SEQ ID NO: 56 (LCDR1), SEQ ID NO: 59 (LCDR2), SEQ ID NO: 62 (LCDR3).

In some embodiment, the binding protein comprises (a) V_{H1} and V_{L}u corresponds to the amino acid sequences of SEQ ID NOS: 11 and 3 respectively, and/or (b) V_{H2} and V_{L2} corresponds to the amino acid sequences of SEQ ID NOS: 93 and 95 respectively, as shown hereinafter.

V_{H1}
(SEQ ID NO: 11)
EVQLVESGGG LVQPDRSLRL SCAASGFTFH DYAMHWVRQA PGKGLEWVST
ISWNSGTIGY ADSVKGRFTI SRDNAKNSLY LQMNSLRAED TALYYCAKDI
QYGNYYYGMD VWGQGTTVTV SS
V_{L1}
(SEQ ID NO: 3)
EIVLTQSPAT LSLSPGERAT LSCRASQSVS SYLAWYQQKP GQAPRLLIYD
ASNRATGIPA RFSGSGSGTD FTLTISSLEP EDFAVYYCQQ RSNWPITFGQ GTRLEIK
V_{H2}
(SEQ ID NO: 93)
QVQLVQSGAE VKKPGSSVKV SCKASGYTFS DYVINWVRQA PGQGLEWMGE
IYPGSGTNYY NEKFKAKATI TADKSTSTAY MELSSLRSED TAVYYCARRG
RYGLYAMDYW GQGTTVTVSS
V_{L2}
(SEQ ID NO: 95)
DIQMTQSPSS LSASVGDRVT ITCRASQDIS NYLNWYQQKP GKAPKLLIYY
TSRLHSGVPS RFSGSGSGTD FTFTISSLQP EDIATYFCQQ GNTRPWTFGG GTKVEIK

In some embodiment, the binding protein comprises (a) V_{H1} and V_{L}u corresponds to the amino acid sequences of SEQ ID NOS: 11 and 3 respectively or a variant thereof with at least 95% of sequence identity, and/or (b) V_{H2} and VL2 corresponds to the amino acid sequences of SEQ ID NOS: 93 and 95 respectively or a variant thereof with at least 95% of sequence identity.

In some embodiment, the binding protein comprises (a) V_{H1} and V_{L}u corresponds to the amino acid sequences of SEQ ID NOS: 11 and 3 respectively or a variant thereof with at least 90% of sequence identity, and/or (b) V_{H2} and VL2 corresponds to the amino acid sequences of SEQ ID NOS: 93 and 95 respectively or a variant thereof with at least 90% of sequence identity.

In some embodiments, in the heterotrimeric binding protein of the disclosure:
CH1 is an immunoglobulin heavy chain constant domain 1 that comprises the amino acid sequence of SEQ ID NO: 12;
CK is an immunoglobulin kappa light chain constant domain (CK) that comprises the amino acid sequence of SEQ ID NO: 4;
(Fc domain)A comprises a CH2-CH3 domains corresponding to the amino acid sequence of SEQ ID NO: 6;
(Fc domain)B comprises a CH2-CH3 domains corresponding to the amino acid sequence of SEQ ID NO: 14;
Hinge1 corresponds to the amino acid sequence of SEQ ID NO: 5;
Hinge2 corresponds to the amino acid sequence of SEQ ID NO: 13;
Hinge3 corresponds to the amino acid sequence of SEQ ID NO: 19;
L1 corresponds to the amino acid sequence of SEQ ID NO: 15; and/or
L2 corresponds to any one of the amino acid sequence of SEQ ID NOS: 20-23.

In some embodiments, the ABD that binds to CD20 and the ABD that binds to NKp46 each have a Fab structure.

In some embodiments, the binding protein comprises a first polypeptide chain (I) comprising an amino acid sequence of SEQ ID NO: 1, a second polypeptide chain (II) comprising an amino acid sequence of SEQ ID NO: 9, and a third polypeptide chain comprising an amino acid sequence of SEQ ID NO: 17, as disclosed hereinafter

First polypeptide chain (I)
(SEQ ID NO: 1)
EIVLTQSPAT LSLSPGERAT LSCRASQSVS SYLAWYQQKP GQAPRLLIYD
ASNRATGIPA RFSGSGSGTD FTLTISSLEP EDFAVYYCQQ RSNWPITFGQ GTRLEIKRTV
AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSFN
RGECDKTHTC PPCPAPELLG GPSVFLFPPK PKDTLMISRT PEVTCVVVDV SHEDPEVKFN
WYVDGVEVHN AKTKPREEQY NSTYRVVSVL TVLHQDWLNG KEYKCKVSNK
ALPAPIEKTI SKAKGQPREP QVYTLPPSRE EMTKNQVSLT CLVKGFYPSD IAVEWESNGQ
PENNYKTTPP VLDSDGSFFL YSKLTVDKSR WQQGNVFSCS VMHEALHNHY
TQKSLSLSPG K
Second polypeptide chain (II)
(SEQ ID NO: 9)
EVQLVESGGG LVQPDRSLRL SCAASGFTFH DYAMHWVRQA PGKGLEWVST
ISWNSGTIGY ADSVKGRFTI SRDNAKNSLY LQMNSLRAED TALYYCAKDI QYGNYYYGMD
VWGQGTTVTV SSASTKGPSV FPLAPSSKST SGGTAALGCL VKDYFPEPVT
VSWNSGALTS GVHTFPAVLQ SSGLYSLSSV VTVPSSSLGT QTYICNVNHK
PSNTKVDKRV EPKSCDKTHT CPPCPAPELL GGPSVFLFPP KPKDTLMISR TPEVTCVVVD
VSHEDPEVKF NWYVDGVEVH NAKTKPREEQ YNSTYRVVSV LTVLHQDWLN
GKEYKCKVSN KALPAPIEKT ISKAKGQPRE PQVYTLPPSR EEMTKNQVSL TCLVKGFYPS
DIAVEWESNG QPENNYKTTP PVLDSDGSFF LYSKLTVDKS RWQQGNVFSC
SVMHEALHNH YTQKSLSLSP GSTGSQVQLV QSGAEVKKPG SSVKVSCKAS
GYTFSDYVIN WVRQAPGQGL EWMGEIYPGS GTNYYNEKFK AKATITADKS
TSTAYMELSS LRSEDTAVYY CARRGRYGLY AMDYWGQGTT VTVSSRTVAA
PSVFIFPPSD EQLKSGTASV VCLLNNFYPR EAKVQWKVDN ALQSGNSQES
VTEQDSKDST YSLSSTLTLS KADYEKHKVY
Third polypeptide chain (III)
(SEQ ID NO: 17)
DIQMTQSPSS LSASVGDRVT ITCRASQDIS NYLNWYQQKP GKAPKLLIYY
TSRLHSGVPS RFSGSGSGTD FTFTISSLQP EDIATYFCQQ GNTRPWTFGG GTKVEIKAST
KGPSVFPLAP SSKSTSGGTA ALGCLVKDYF PEPVTVSWNS GALTSGVHTF
PAVLQSSGLY SLSSVVTVPS SSLGTQTYIC NVNHKPSNTK VDKRVEPKSC
DKTHSGSSSS GSSSSGSSSS TKKTQLQLEH LLLDLQMILN GINNYKNPKL TAMLTKKFYM
PKKATELKHL QCLEEELKPL EEVLNLAQSK NFHLRPRDLI SNINVIVLEL KGSETTFMCE
YADETATIVE FLNRWITFAQ SIISTLT

In some embodiments, the binding protein comprises a first polypeptide chain (I) comprising an amino acid sequence of SEQ ID NO: 1, a second polypeptide chain (II) comprising an amino acid sequence of SEQ ID NO: 9, and a third polypeptide chain comprising an amino acid sequence of SEQ ID NO: 17, or a variant thereof with at least 95% of sequence identity.

In some embodiments, the binding protein comprises a first polypeptide chain (I) comprising an amino acid sequence of SEQ ID NO: 1, a second polypeptide chain (II) comprising an amino acid sequence of SEQ ID NO: 9, and a third polypeptide chain comprising an amino acid sequence of SEQ ID NO: 17, or a variant thereof with at least 90% of sequence identity.

In some embodiments, the binding protein comprises a first polypeptide chain (I) comprising an amino acid sequence of SEQ ID NO: 1, a second polypeptide chain (II) comprising an amino acid sequence of SEQ ID NO: 9, and a third polypeptide chain comprising an amino acid sequence of SEQ ID NO: 17, or a variant thereof with at least 80% of sequence identity.

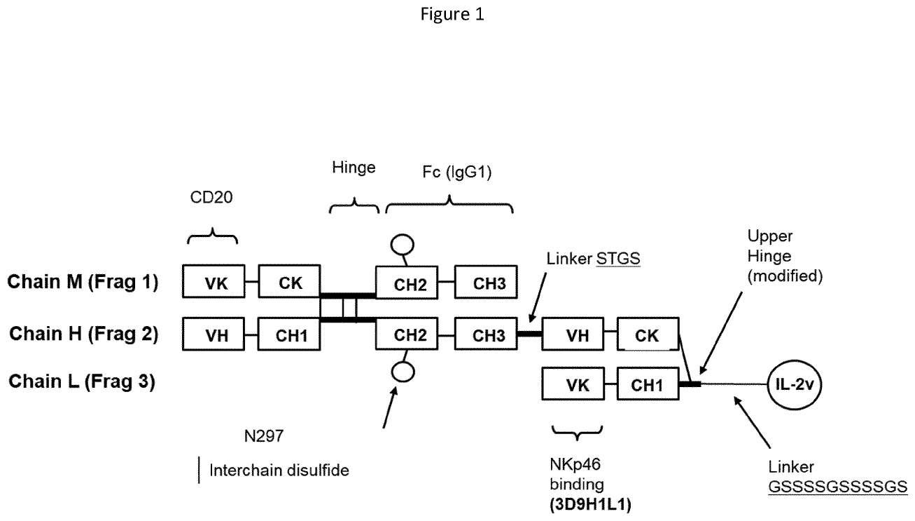

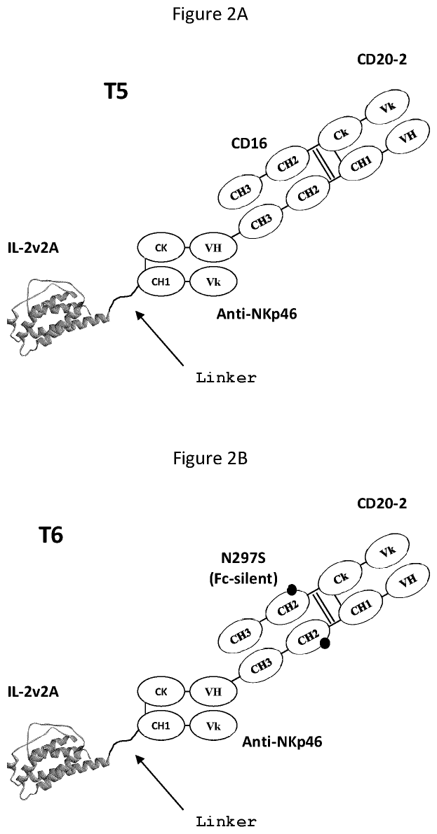

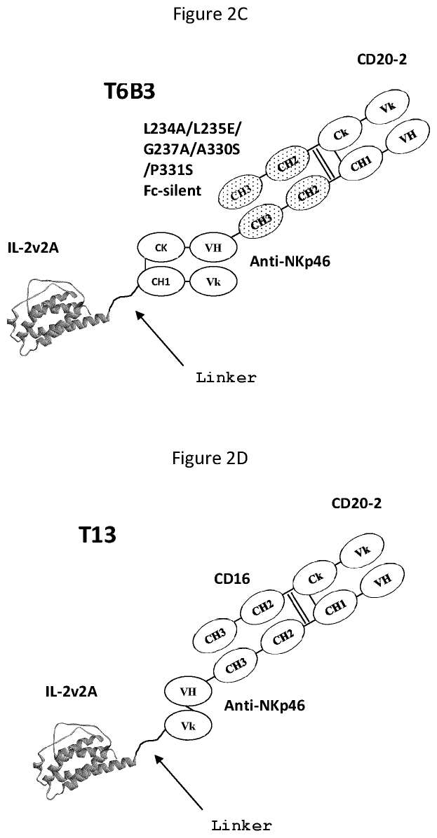

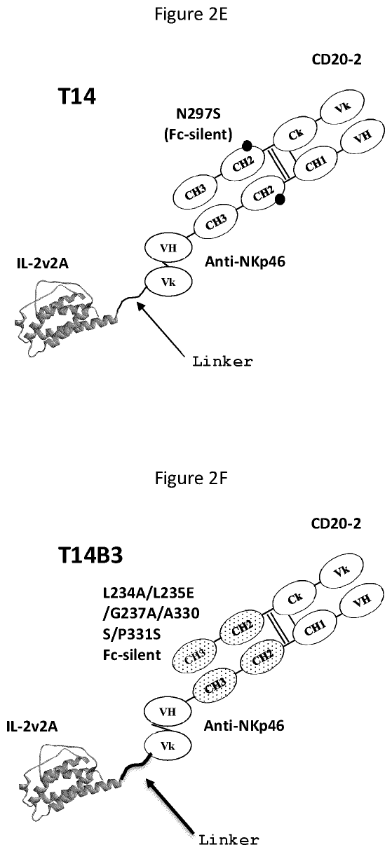

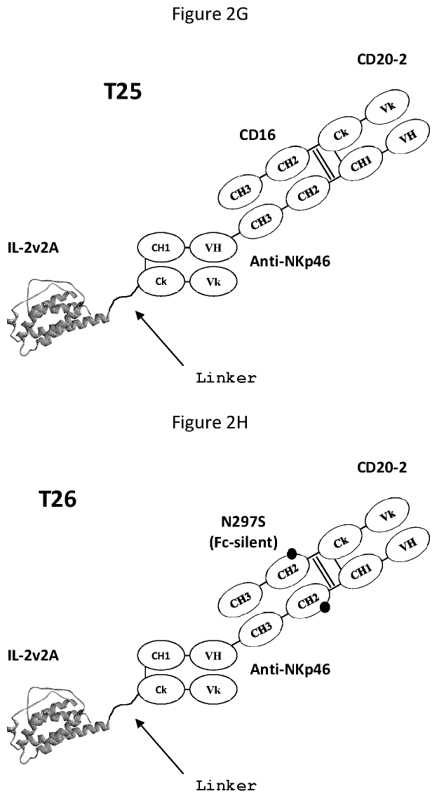

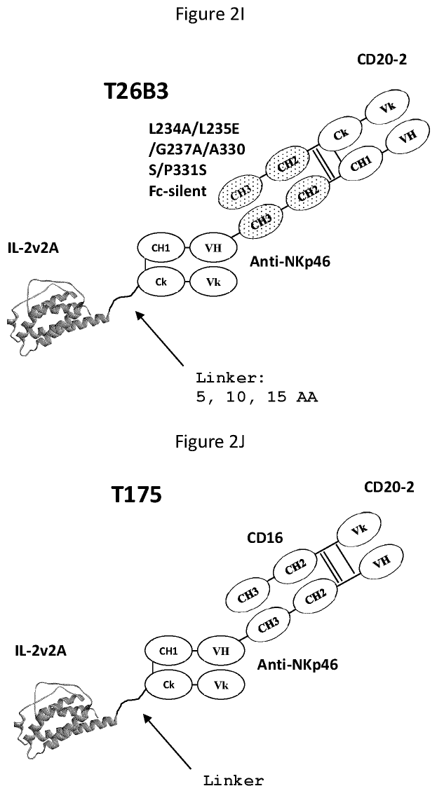

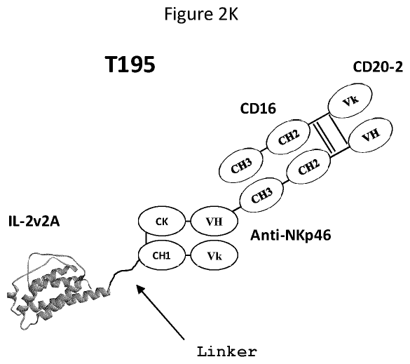

The binding proteins having the above first, second and third polypeptide chains are in a protein format shown in FIGS. 1 and 2A (CD20-2-T5-NKCE4).

In another embodiment, C_{2A} is CH1 and C_{2B} is C_{K}.

In some embodiment, the binding protein comprises a first polypeptide chain (I) comprising an amino acid sequence of SEQ ID NO: 1, a second polypeptide chain (II) comprising an amino acid sequence of SEQ ID NO: 73, and a third polypeptide chain comprising an amino acid sequence of SEQ ID NO: 74.

In some embodiment, the binding protein comprises a first polypeptide chain (I) comprising an amino acid sequence of SEQ ID NO: 1, a second polypeptide chain (II) comprising an amino acid sequence of SEQ ID NO: 73, and a third polypeptide chain comprising an amino acid sequence of SEQ ID NO: 74, or a variant thereof with at least 95% of sequence identity.

In some embodiment, the binding protein comprises a first polypeptide chain (I) comprising an amino acid sequence of SEQ ID NO: 1, a second polypeptide chain (II) comprising an amino acid sequence of SEQ ID NO: 73, and a third polypeptide chain comprising an amino acid sequence of SEQ ID NO: 74, or a variant thereof with at least 90% of sequence identity.

In some embodiment, the binding protein comprises a first polypeptide chain (I) comprising an amino acid sequence of SEQ ID NO: 1, a second polypeptide chain (II) comprising an amino acid sequence of SEQ ID NO: 73, and a third polypeptide chain comprising an amino acid sequence of SEQ ID NO: 74, or a variant thereof with at least 80% of sequence identity.

The binding proteins having the above first, second and third polypeptide chains (in which C_{2A} is CH1 and C_{2B} is C_{K}) are arranged in format shown in FIG. 2G (CD20-2-T25-NKCE4).

In another embodiment, the binding protein of the disclosure has a residue N297 (according to Kabat numbering) of Fc domains mutated to prevent said residue to be glycosylated. In a preferred embodiment, said mutation is a N297S substitution. Advantageously, said mutation substantially abolish CD16A binding.

According to some embodiment, C2A is CK and C2B is CH1.

In some embodiment, the binding protein comprises a first polypeptide chain (I) comprising an amino acid sequence of SEQ ID NO: 66, a second polypeptide chain (II) comprising an amino acid sequence of SEQ ID NO: 67, and a third polypeptide chain comprising an amino acid sequence of SEQ ID NO: 17.

In some embodiment, the binding protein comprises a first polypeptide chain (I) comprising an amino acid sequence of SEQ ID NO: 66, a second polypeptide chain (II) comprising an amino acid sequence of SEQ ID NO: 67, and a third polypeptide chain comprising an amino acid sequence of SEQ ID NO: 17, or a variant thereof with at least 95% of sequence identity.

In some embodiment, the binding protein comprises a first polypeptide chain (I) comprising an amino acid sequence of SEQ ID NO: 66, a second polypeptide chain (II) comprising an amino acid sequence of SEQ ID NO: 67, and a third polypeptide chain comprising an amino acid sequence of SEQ ID NO: 17, or a variant thereof with at least 90% of sequence identity.

In some embodiment, the binding protein comprises a first polypeptide chain (I) comprising an amino acid sequence of SEQ ID NO: 66, a second polypeptide chain (II) comprising an amino acid sequence of SEQ ID NO: 67, and a third polypeptide chain comprising an amino acid sequence of SEQ ID NO: 17, or a variant thereof with at least 80% of sequence identity.

An example of such protein format is presented in FIG. 2B (CD20-2-T6-NKCE4).

In an alternative embodiment, C_{2A} is CH1 and C_{2B} is C_{K}.

In some embodiment, the binding protein comprises a first polypeptide chain (I) comprising an amino acid sequence of SEQ ID NO: 66, a second polypeptide chain (II) comprising an amino acid sequence of SEQ ID NO: 75, and a third polypeptide chain comprising an amino acid sequence of SEQ ID NO: 74.

In some embodiment, the binding protein comprises a first polypeptide chain (I) comprising an amino acid sequence of SEQ ID NO: 66, a second polypeptide chain (II) comprising an amino acid sequence of SEQ ID NO: 75, and a third polypeptide chain comprising an amino acid sequence of SEQ ID NO: 74, or a variant thereof with at least 95% of sequence identity.

In some embodiment, the binding protein comprises a first polypeptide chain (I) comprising an amino acid sequence of SEQ ID NO: 66, a second polypeptide chain (II) comprising an amino acid sequence of SEQ ID NO: 75, and a third polypeptide chain comprising an amino acid sequence of SEQ ID NO: 74, or a variant thereof with at least 90% of sequence identity.

In some embodiment, the binding protein comprises a first polypeptide chain (I) comprising an amino acid sequence of SEQ ID NO: 66, a second polypeptide chain (II) comprising an amino acid sequence of SEQ ID NO: 75, and a third polypeptide chain comprising an amino acid sequence of SEQ ID NO: 74, or a variant thereof with at least 80% of sequence identity.

An example of such protein format is presented in FIG. 2H (CD20-2-T26-NKCE4).

In another embodiment, the binding protein has Fc domains comprising L234A, L235E, G237A, A330S and/or P331S substitutions according to kabat numbering.

According to some embodiment, C2A is CK and C2B is CH1.

In some embodiment, the binding protein comprises a first polypeptide chain (I) comprising an amino acid sequence of SEQ ID NO: 68, a second polypeptide chain (II) comprising an amino acid sequence of SEQ ID NO: 69, and a third polypeptide chain comprising an amino acid sequence of SEQ ID NO: 17.

In some embodiment, the binding protein comprises a first polypeptide chain (I) comprising an amino acid sequence of SEQ ID NO: 68, a second polypeptide chain (II) comprising an amino acid sequence of SEQ ID NO: 69, and a third polypeptide chain comprising an amino acid sequence of SEQ ID NO: 17, or a variant thereof with at least 95% of sequence identity.

In some embodiment, the binding protein comprises a first polypeptide chain (I) comprising an amino acid sequence of SEQ ID NO: 68, a second polypeptide chain (II) comprising an amino acid sequence of SEQ ID NO: 69, and a third polypeptide chain comprising an amino acid sequence of SEQ ID NO: 17, or a variant thereof with at least 90% of sequence identity.

In some embodiment, the binding protein comprises a first polypeptide chain (I) comprising an amino acid sequence of SEQ ID NO: 68, a second polypeptide chain (II) comprising an amino acid sequence of SEQ ID NO: 69, and a third polypeptide chain comprising an amino acid sequence of SEQ ID NO: 17, or a variant thereof with at least 80% of sequence identity.

An example of such protein format is presented in FIG. 2C (CD20-2-T6B3-NKCE4).

In an alternative embodiment, C2A is CH1 and C2B is CK.

In some embodiment, the binding protein comprises a first polypeptide chain (I) comprising an amino acid sequence of SEQ ID NO: 68, a second polypeptide chain (II) comprising an amino acid sequence of SEQ ID NO: 76, and a third polypeptide chain comprising an amino acid sequence of SEQ ID NO: 74.

In some embodiment, the binding protein comprises a first polypeptide chain (I) comprising an amino acid sequence of SEQ ID NO: 68 or a variant thereof with at least 95% of sequence identity, a second polypeptide chain (II) comprising an amino acid sequence of SEQ ID NO: 76 or a variant thereof with at least 95% of sequence identity, and a third polypeptide chain comprising an amino acid sequence of SEQ ID NO: 74 or a variant thereof with at least 95% of sequence identity.

In some embodiment, the binding protein comprises a first polypeptide chain (I) comprising an amino acid sequence of SEQ ID NO: 68 or a variant thereof with at least 90% of sequence identity, a second polypeptide chain (II) comprising an amino acid sequence of SEQ ID NO: 76 or a variant thereof with at least 90% of sequence identity, and a third polypeptide chain comprising an amino acid sequence of SEQ ID NO: 74 or a variant thereof with at least 90% of sequence identity, or a variant thereof with at least 90% of sequence identity.

In some embodiment, the binding protein comprises a first polypeptide chain (I) comprising an amino acid sequence of SEQ ID NO: 68 or a variant thereof with at least 80% of sequence identity, a second polypeptide chain (II) comprising an amino acid sequence of SEQ ID NO: 76 or a variant thereof with at least 80% of sequence identity, and a third polypeptide chain comprising an amino acid sequence of SEQ ID NO: 74 or a variant thereof with at least 80% of sequence identity.

An example of such protein format is presented in FIG. 2I (CD20-2-T26B3-NKCE4).

In other embodiments, the binding protein of the disclosure comprises an ABD that binds to CD20 that is a VH/VL pair and an ABD that binds to NKp46 that is a Fab.

According to some embodiment, C2A is CK and C2B is CH1.

In some embodiment, the binding protein comprises a first polypeptide chain (I) comprising an amino acid sequence of SEQ ID NO: 77, a second polypeptide chain (II) comprising an amino acid sequence of SEQ ID NO: 79, and a third polypeptide chain comprising an amino acid sequence of SEQ ID NO: 17.

In some embodiment, the binding protein comprises a first polypeptide chain (I) comprising an amino acid sequence of SEQ ID NO: 77 or a variant thereof with at least 90% of sequence identity, a second polypeptide chain (II) comprising an amino acid sequence of SEQ ID NO: 79 or a variant thereof with at least 95% of sequence identity, and a third polypeptide chain comprising an amino acid sequence of SEQ ID NO: 17 or a variant thereof with at least 95% of sequence identity.

In some embodiment, the binding protein comprises a first polypeptide chain (I) comprising an amino acid sequence of SEQ ID NO: 77 or a variant thereof with at least 90% of sequence identity, a second polypeptide chain (II) comprising an amino acid sequence of SEQ ID NO: 79 or a variant thereof with at least 90% of sequence identity, and a third polypeptide chain comprising an amino acid sequence of SEQ ID NO: 17 or a variant thereof with at least 90% of sequence identity.

In some embodiment, the binding protein comprises a first polypeptide chain (I) comprising an amino acid sequence of SEQ ID NO: 77 or a variant thereof with at least 80% of sequence identity, a second polypeptide chain (II) comprising an amino acid sequence of SEQ ID NO: 79 or a variant thereof with at least 80% of sequence identity, and a third polypeptide chain comprising an amino acid sequence of SEQ ID NO: 17 or a variant thereof with at least 80% of sequence identity, or a variant thereof with at least 80% of sequence identity.

An example of such protein format is presented in FIG. 2K (CD20-2-T195-NKCE4).

In an alternative embodiment, C_{2A} is CH1 and C_{2B} is C_{K}.

In some embodiment, the binding protein comprises a first polypeptide chain (I) comprising an amino acid sequence of SEQ ID NO: 77, a second polypeptide chain (II) comprising an amino acid sequence of SEQ ID NO: 78, and a third polypeptide chain comprising an amino acid sequence of SEQ ID NO: 74.

In some embodiment, the binding protein comprises a first polypeptide chain (I) comprising an amino acid sequence of SEQ ID NO: 77, a second polypeptide chain (II) comprising an amino acid sequence of SEQ ID NO: 78, and a third polypeptide chain comprising an amino acid sequence of SEQ ID NO: 74, or a variant thereof with at least 95% of sequence identity.

In some embodiment, the binding protein comprises a first polypeptide chain (I) comprising an amino acid sequence of SEQ ID NO: 77 or a variant thereof with at least 90% of sequence identity, a second polypeptide chain (II) comprising an amino acid sequence of SEQ ID NO: 78 or a variant thereof with at least 90% of sequence identity, and a third polypeptide chain comprising an amino acid sequence of SEQ ID NO: 74 or a variant thereof with at least 90% of sequence identity.

In some embodiment, the binding protein comprises a first polypeptide chain (I) comprising an amino acid sequence of SEQ ID NO: 77 or a variant thereof with at least 80% of sequence identity, a second polypeptide chain (II) comprising an amino acid sequence of SEQ ID NO: 78 or a variant thereof with at least 80% of sequence identity, and a third polypeptide chain comprising an amino acid sequence of SEQ ID NO: 74 or a variant thereof with at least 80% of sequence identity.

An example of such protein format is presented in FIG. 2J (CD20-2-T175-NKCE4).

In another embodiment, the binding protein of the disclosure is heterodimeric and comprises two polypeptide chains (I) and (II) that form two ABDs, as defined above:

V_{1A}-C_{1A}-Hinge₁-(Fc domain)_{A} (I)

V_{1B}-C_{1B}-Hinge₂-(Fc domain)_{B}-L1-V_{2A}-L2-V_{2B}-L3-IL-2 (II)
wherein:
V_{1A} and V_{1B} form a binding pair V₁ (V_{H1}/V_{L1});
V_{2A} and V_{2B} form an scFv;
C_{1A} and C_{1B} form a pair C₁ (CH1/C_{L}) and C_{2A} and C_{2B} form a pair C₂ (CH1/C_{L}) wherein CH1 is an immunoglobulin heavy chain constant domain 1 and C_{L} is an immunoglobulin light chain constant domain;
Hinge₁, Hinge₂ and Hinge₃ are identical or different and correspond to all or part of an immunoglobulin hinge region;
(Fc domain)_{A} and (Fc domain)_{B} are identical or different, and comprise a CH2-CH3 domain;
L₁ and L₂ are an amino acid linker, wherein L₁ and L₂ can be different or the same;
IL-2 is a variant human interleukin-2 polypeptide or portion thereof that binds to CD122 present on NK cells. In one embodiment, binding pair V₁ binds CD20 and binding pair V₂ binds NKp46.

In some embodiment, the binding protein comprises a first polypeptide chain (I) comprising an amino acid sequence of SEQ ID NO: 1 (disclosed hereinafter), and a second polypeptide chain (II) comprising an amino acid sequence of SEQ ID NO: 70 (disclosed hereinafter.

First polypeptide chain (I)
(SEQ ID NO: 1)
EIVLTQSPAT LSLSPGERAT LSCRASQSVS SYLAWYQQKP GQAPRLLIYD
ASNRATGIPA RFSGSGSGTD FTLTISSLEP EDFAVYYCQQ RSNWPITFGQ GTRLEIKRTV
AAPSVFIFPP SDEQLKSGTA SVVCLLNNFY PREAKVQWKV DNALQSGNSQ
ESVTEQDSKD STYSLSSTLT LSKADYEKHK VYACEVTHQG LSSPVTKSFN
RGECDKTHTC PPCPAPELLG GPSVFLFPPK PKDTLMISRT PEVTCVVVDV SHEDPEVKFN
WYVDGVEVHN AKTKPREEQY NSTYRVVSVL TVLHQDWLNG KEYKCKVSNK
ALPAPIEKTI SKAKGQPREP QVYTLPPSRE EMTKNQVSLT CLVKGFYPSD IAVEWESNGQ
PENNYKTTPP VLDSDGSFFL YSKLTVDKSR WQQGNVFSCS VMHEALHNHY
TQKSLSLSPG K
Second polypeptide chain (II)
(SEQ ID NO: 70)
EVQLVESGGG LVQPDRSLRL SCAASGFTFH DYAMHWVRQA PGKGLEWVST
ISWNSGTIGY ADSVKGRFTI SRDNAKNSLY LQMNSLRAED TALYYCAKDI QYGNYYYGMD
VWGQGTTVTV SSASTKGPSV FPLAPSSKST SGGTAALGCL VKDYFPEPVT
VSWNSGALTS GVHTFPAVLQ SSGLYSLSSV VTVPSSSLGT QTYICNVNHK
PSNTKVDKRV EPKSCDKTHT CPPCPAPELL GGPSVFLFPP KPKDTLMISR TPEVTCVVVD
VSHEDPEVKF NWYVDGVEVH NAKTKPREEQ YNSTYRVVSV LTVLHQDWLN
GKEYKCKVSN KALPAPIEKT ISKAKGQPRE PQVYTLPPSR EEMTKNQVSL TCLVKGFYPS
DIAVEWESNG QPENNYKTTP PVLDSDGSFF LYSKLTVDKS RWQQGNVFSC
SVMHEALHNH YTQKSLSLSP GSTGSQVQLV QSGAEVKKPG SSVKVSCKAS
GYTFSDYVIN WVRQAPGQGL EWMGEIYPGS GTNYYNEKFK AKATITADKS
TSTAYMELSS LRSEDTAVYY CARRGRYGLY AMDYWGQGTT VTVSSVEGGS
GGSGGSGGSG GVDDIQMTQS PSSLSASVGD RVTITCRASQ DISNYLNWYQ
QKPGKAPKLL IYYTSRLHSG VPSRFSGSGS GTDFTFTISS LQPEDIATYF CQQGNTRPWT
FGGGTKVEIK GSSSSGSSSS GSSSSTKKTQ LQLEHLLLDL QMILNGINNY KNPKLTAMLT
KKFYMPKKAT ELKHLQCLEE ELKPLEEVLN LAQSKNFHLR PRDLISNINV IVLELKGSET
TFMCEYADET ATIVEFLNRW ITFAQSIIST LT

In some embodiment, the binding protein comprises a first polypeptide chain (I) comprising an amino acid sequence of SEQ ID NO: 1 or a variant thereof with at least 95% of sequence identity, and a second polypeptide chain (II) comprising an amino acid sequence of SEQ ID NO: 70 or a variant thereof with at least 95% of sequence identity.

In some embodiment, the binding protein comprises a first polypeptide chain (I) comprising an amino acid sequence of SEQ ID NO: 1 or a variant thereof with at least 90% of sequence identity, and a second polypeptide chain (II) comprising an amino acid sequence of SEQ ID NO: 70, or a variant thereof with at least 90% of sequence identity.

In some embodiment, the binding protein comprises a first polypeptide chain (I) comprising an amino acid sequence of SEQ ID NO: 1 or a variant thereof with at least 80% of sequence identity, and a second polypeptide chain (II) comprising an amino acid sequence of SEQ ID NO: 70, or a variant thereof with at least 80% of sequence identity.

An example of such protein format is presented in FIG. 2D (CD20-2-T13-NKCE4).

In another embodiment, the binding protein of the disclosure has a residue N297 (according to Kabat numbering) of Fc domains mutated to prevent said residue to be glycosylated. In a preferred embodiment, said mutation is a N297S substitution. Advantageously, said mutation substantially abolish CD16A binding.

In some embodiment, the binding protein comprises a first polypeptide chain (I) comprising an amino acid sequence of SEQ ID NO: 66, and a second polypeptide chain (II) comprising an amino acid sequence of SEQ ID NO: 71.

In some embodiment, the binding protein comprises a first polypeptide chain (I) comprising an amino acid sequence of SEQ ID NO: 66 or a variant thereof with at least 95% of sequence identity, and a second polypeptide chain (II) comprising an amino acid sequence of SEQ ID NO: 71 or a variant thereof with at least 95% of sequence identity.

In some embodiment, the binding protein comprises a first polypeptide chain (I) comprising an amino acid sequence of SEQ ID NO: 6 or a variant thereof with at least 90% of sequence identity, and a second polypeptide chain (II) comprising an amino acid sequence of SEQ ID NO: 71 or a variant thereof with at least 90% of sequence identity.

In some embodiment, the binding protein comprises a first polypeptide chain (I) comprising an amino acid sequence of SEQ ID NO: 66 or a variant thereof with at least 80% of sequence identity, and a second polypeptide chain (II) comprising an amino acid sequence of SEQ ID NO: 71 or a variant thereof with at least 80% of sequence identity.

An example of such protein format is presented in FIG. 2E (CD20-2-T14-NKCE4).

In another embodiment, the binding protein has Fc domains comprising L234A, L235E, G237A, A330S and/or P331S substitutions according to kabat numbering.

In some embodiment, the binding protein comprises a first polypeptide chain (I) comprising an amino acid sequence of SEQ ID NO: 68, and a second polypeptide chain (II) comprising an amino acid sequence of SEQ ID NO: 72.

In some embodiment, the binding protein comprises a first polypeptide chain (I) comprising an amino acid sequence of SEQ ID NO: 68 or a variant thereof with at least 95% of sequence identity, and a second polypeptide chain (II) comprising an amino acid sequence of SEQ ID NO: 72 or a variant thereof with at least 95% of sequence identity.

In some embodiment, the binding protein comprises a first polypeptide chain (I) comprising an amino acid sequence of SEQ ID NO: 68 or a variant thereof with at least 90% of sequence identity, and a second polypeptide chain (II) comprising an amino acid sequence of SEQ ID NO: 72, or a variant thereof with at least 90% of sequence identity.

In some embodiment, the binding protein comprises a first polypeptide chain (I) comprising an amino acid sequence of SEQ ID NO: 68 or a variant thereof with at least 80% of sequence identity, and a second polypeptide chain (II) comprising an amino acid sequence of SEQ ID NO: 72, or a variant thereof with at least 80% of sequence identity.

An example of such protein format is presented in FIG. 2F (CD20-2-T14B3-NKCE4).

### CD20 ABD

CD20 is a cell surface protein present on most B-cell neoplasms, and absent on otherwise similar appearing T-cell neoplasms. CD20 positive cells are also sometimes found in cases of Hodgkin's disease, myeloma, and thymoma. CD20 is the target of the monoclonal antibodies (mAb) rituximab, ofatumumab, ocrelizumab, genmab, obinutuzumab, Ibritumomab tiuxetan, AME-133v, IMMU-106, TRU-015, and tositumomab, which are all active agents in the treatment of all B cell lymphomas and leukemias.

In one embodiment, the ABD that binds to a CD20 polypeptide of a binding protein of the disclosure comprises a VH and VL pair presented hereinafter in Table 2:

TABLE 2

| | | |
|---|---|---|
| SEQ ID | CD20-2 | EVQLVESGGG LVQPDRSLRL SCAASGFTFH DYAMHWVRQA |
| NO: 11 | VH | PGKGLEWVST ISWNSGTIGY QYGNYYYGMD SRDNAKNSLY |
| | | LQMNSLRAED TALYYCAKDI ADSVKGRFTI VWGQGTTVTV |
| | | SS |
| SEQ ID | CD20-2 | EIVLTQSPAT LSLSPGREAT LSCRASQSVS SYLAWYQQKP |
| NO: 3 | VL | GQAPRLLIYD ASNRATGIPA RFSGSGSGTF TLTISSLEPE |
| | | DFAVYYCQQR SNWPITFGQG TRLEIK |

In one embodiment, the ABD that binds to a 0020 polypeptide of a binding protein of the disclosure comprises a VH comprising three CDRs (HCDR1, HCDR2 and HCDR3) and a VL comprising three CDRs (LCDR1, LCDR2 and LCDR3).

In another aspect of any of the embodiments herein, any of the CDR1, CDR2 and CDR3 of the heavy and light chains may be characterized by a sequence of at least 4, 5, 6, 7, 8, 9 or 10 contiguous amino acids thereof, and/or as having an amino acid sequence that shares at least 50%, 60%, 70%, 80%, 85%, 90% or 95% sequence identity with the particular CDR or set of CDRs listed in the corresponding SEQ ID NO or table hereinafter, that summarize the sequences of the CDRs, according to IMGT, Kabat and Chothia definitions systems:

TABLE 3

| | CDR | SEQ | | SEQ | | SEQ | |
|---|---|---|---|---|---|---|---|
| mAb | definition | ID | Sequence | ID | Sequence | ID | Sequence |
| | | HCDR1 | | HCDR2 | | HCDR3 | |
| CD20-2 | Kabat | 29 | DYAMH | 32 | TISWNSGTIGYADSV | 35 | DIQYGNYYYG |
| | | | | | KG | | MDV |
| | Chothia | 30 | GFTFHDY | 33 | WNSG | 36 | IQYGNYYYG |
| | | | | | | | MD |
| | IMGT | 31 | GFTFHDYA | 34 | ISWNSGTI | 37 | AKDIQYGNYY |
| | | | | | | | YGMDV |
| | | LCDR1 | | LCDR2 | | LCDR3 | |
| | Kabat | 38 | RASQSVSS | 41 | DASNRAT | 44 | QQRSNWPIT |
| | | | YLA | | | | |
| | Chothia | 39 | SQSVSSY | 42 | DAS | 45 | RSNWPI |
| | IMGT | 40 | QSVSSY | 43 | DAS | 46 | QQRSNWPIT |

In some embodiments, the first ABD of the binding protein specifically binds to a human 0020 polypeptide and comprises:
a VH1 comprising a CDR1, CDR2 and CDR3 corresponding to the amino acid sequences of SEQ ID NO: 29 (HCDR1), SEQ ID NO: 32 (HCDR2), SEQ ID NO: 35 (HCDR3), and
a VL1 comprising a CDR1, CDR2 and CDR3 corresponding to the amino acid sequences of SEQ ID NO: 38 (LCDR1), SEQ ID NO: 41 (LCDR2), SEQ ID NO: 44 (LCDR3).

### NKp46

As discussed herein, the binding protein of the disclosure comprises and ABD that binds to a human NKp46 polypeptide.

In one embodiment, the second ABD of the binding protein comprises a VH comprising CDR 1, 2 and 3 of amino acid sequences of SEQ ID NO: 47 (HCDR1), SEQ ID NO: 50 (HCDR2), SEQ ID NO: 53 (HCDR3), optionally wherein one, two, three or more amino acids in a CDR may be substituted by a different amino acid; and a VL comprising CDR 1, 2 and 3 of amino acid sequences of SEQ ID NO: 56 (LCDR1), SEQ ID NO: 59 (LCDR2), SEQ ID NO: 62 (LCDR3) optionally wherein one, two, three or more amino acids in a CDR may be substituted by a different amino acid.

Accordingly, said second ABD of the binding protein of the disclosure can bind a region spanning the D1 and D2 domains (at the border of the D1 and D2 domains, the D1/D2 junction), of the NKp46 polypeptide of SEQ ID NO: 1. In some embodiments, the VH/VL pair of the second ABD of the binding protein have an affinity for human NKp46, as a full-length IgG antibody, characterized by a K_{D} of less than 10⁻⁸ M, less than 10⁻⁹ M, or less than 10⁻¹⁰ M. In some embodiments, the multispecific proteins have an affinity (KD) for human NKp46 of between 1 and 100 nM, optionally between 1 and 50 nM, optionally between 1 and 20 nM, optionally about 10 or 15 nM, as determined by SPR.

In one embodiment, the multispecific protein (or a NKp46-binding ABD or VH/VL pair thereof, for example as when configured in the multispecific protein or as a conventional full-length antibody) binds NKp46 at substantially the same region, site or epitope on NKp46 as antibody NKp46-1. In one embodiment, all key residues of the epitope are in a segment corresponding to domain D1 or D2. In one embodiment, the antibody or multispecific protein binds a residue present in the D1 domain as well as a residue present in in the D2 domain. In one embodiment, the antibodies bind an epitope comprising 1, 2, 3, 4, 5, 6, 7 or more residues in the segment corresponding to D1/D2 junction of the NKp46 polypeptide of SEQ ID NO: 88. In one embodiment, the antibodies or multispecific proteins bind NKp46 at the D1/D2 domain junction and bind an epitope comprising or consisting of 1, 2, 3, 4 or 5 of the residues K41, E42, E119, Y121 and/or Y194.

The amino acid sequence of the heavy chain variable region and the amino acid sequence of the light chain variable region of NKp46-1, is presented in table 4 hereinafter.

TABLE 4

| | | |
|---|---|---|
| NKp46-1 VH | SEQ ID | QVQLQQSGPELVKPGASVKMSCKASGYTFTDYVINWGKQRSG |
| | NO: 16 | QGLEWIGEIYPGSGTNYYNEKFKAKATLTADKSSNIAYMQLSSLT |
| | | SEDSAVYFCARRGRYGLYAMDYWGQGTSVTVSS |
| NKp46-1 VL | SEQ ID | DIQMTQTTSSLSASLGDRVTISCRASQDISNYLNWYQQKPDGTV |
| | NO: 18 | KLLIYYTSRLHSGVPSRFSGSGSGTDYSLTINNLEQEDIATYFCQ |
| | | QGNTRPWTFGGGTKLEIK |

A NKp46-binding multispecific protein that binds essentially the same epitope or determinant as monoclonal antibody NKp46-1, optionally the antibody comprises a hypervariable region of antibody NKp46-1. In any of the embodiments herein, antibody NKp46-1 can be characterized by its amino acid sequence and/or nucleic acid sequence encoding it. In one embodiment, the antibody comprises the Fab or F(ab′)₂ portion of NKp46-1.

In one embodiment, the NKp46 binding ABD comprises humanized VH/VL of antibody NKp46-1. Based on 3D modelling studies, different heavy and light chain variable regions were designed that included NKp46-1 CDRs and human frameworks, produced as human IgG1 antibodies, and tested for binding to cynomolgus NKp46. Two combinations of heavy and light chains were able to bind to both human and cynomolgus NKp46: the heavy chain variable region “H1” and the heavy chain “H3”, in each case combined with the light chain “L1”. These cross-binding variable regions included, for the heavy chain variable region: the NKp46-1 heavy chain CDRs (shown below, underlined), human IGHV1-69*06 gene framework 1, 2 and 3 regions and a human IGHJ6*01 gene framework 4 region. The light chain variable region: the NKp46-1 light chain CDRs (shown below, underlined), human IGKV1-33*01 gene framework 1, 2 and 3 regions and a human IGKJ4*01 gene framework 4 region. CDRs were chosen according to Kabat numbering. The H1, H3 and L1 chain had the specific amino acid substitutions (shown in bold and underlining below). L1 had a phenylalanine at Kabat light chain residue 87. H1 had a tyrosine at Kabat heavy chain residue 27 and a lysine and alanine at Kabat residues 66 and 67, respectively. H3 additionally had a glycine at Kabat residue 37, an isoleucine at Kabat residue 48, and a phenylalanine at Kabat residue 91.

TABLE 5

| NKp46-1: “H1” | SEQ | QVQLVQSGAE VKKPGSSVKV SCKASGYTFS |
|---|---|---|
| heavy chain | ID | DYVINWVRQA PGQGLEWMGE IYPGSGTNYY |
| variable | NO: | NEKFKAKATI TADKSTSTAY MELSSLRSED |
| region | 93 | TAVYYCARRG RYGLYAMDYW GQGTTVTVSS |
| NKp46-1: “H3” | SEQ | QVQLVQSGAE VKKPGSSVKV SCKASGYTFT |
| heavy chain | ID | DYVINWGRQA PGQGLEWIGE IYPGSGTNYY |
| variable | NO: | NEKFKAKATI TADKSTSTAY MELSSLRSED |
| region | 94 | TAVYFCARRG RYGLYAMDYW GQGTTVTVSS |
| NKp46-1: “L1” | SEQ | DIQMTQSPSS LSASVGDRVT ITCRASQDIS |
| light chain | ID | NYLNWYQQKP GKAPK LLIYY TSRLHSGVPS |
| variable | NO: | RFSGSGSGTD FTFTISSLQP EDIATYFCQQ |
| region | 95 | GNTRPWTFGG GTKVEIK |

According to one embodiment, an antibody comprises the three CDRs of the heavy chain variable region of NKp46-1, or humanized version thereof (NKp46-1H1 or NKp46-1H3). Also provided is a polypeptide that further comprises one, two or three of the CDRs of the light chain variable region of NKp46-1 or humanized version thereof (NKp46-1L1 or NKp46-1L1). Optionally any one or more of said light or heavy chain CDRs may contain one, two, three, four or five or more amino acid modifications (e.g. substitutions, insertions or deletions).

A multispecific protein or NKp46-binding ABD can for example comprise:
(a) the heavy chain variable region of NKp46-1 (SEQ ID NO: 16) optionally wherein one, two, three or more amino acids may be substituted by a different amino acid;
(b) the light chain variable region NKp46-1 (SEQ ID NO: 18), optionally wherein one, two, three or more amino acids may be substituted by a different amino acid;
or,
(a) the heavy chain variable region of NKp46-1H1 (SEQ ID NO: 93) optionally wherein one, two, three or more amino acids may be substituted by a different amino acid;
(b) the light chain variable region NKp46-1 L1 (SEQ ID NO: 95), optionally wherein one, two, three or more amino acids may be substituted by a different amino acid;
or,
(a) the heavy chain variable region of NKp46-1H3 (SEQ ID NO: 94) optionally wherein one, two, three or more amino acids may be substituted by a different amino acid;
(b) the light chain variable region NKp46-1 L1 (SEQ ID NO: 95), optionally wherein one, two, three or more amino acids may be substituted by a different amino acid.

In some embodiment, the multispecific protein or NKp46-binding ABD can comprise:
(a) the heavy chain CDR 1, 2 and 3 (HCDR1, HCDR2, HCDR3) amino acid sequence of NKp46-1, as shown in the table hereinafter, optionally wherein one, two, three or more amino acids in a CDR may be substituted by a different amino acid;
(b) the light chain CDR 1, 2 and 3 (LCDR1, LCDR2, LCDR3) amino acid sequence of NKp46-1 as shown in the table hereinafter, optionally wherein one, two, three or more amino acids in a CDR may be substituted by a different amino acid;

In one embodiment, the aforementioned CDRs are according to Kabat, e.g. as shown in the table hereinafter. In one embodiment, the aforementioned CDRs are according to Chothia numbering, e.g. as shown in the table hereinafter. In one embodiment, the aforementioned CDRs are according to IMGT numbering, e.g. as shown in the table hereinafter.

In another aspect of any of the embodiments herein, any of the CDR1, CDR2 and CDR3 of the heavy and light chains may be characterized by a sequence of at least 4, 5, 6, 7, 8, 9 or 10 contiguous amino acids thereof, and/or as having an amino acid sequence that shares at least 50%, 60%, 70%, 80%, 85%, 90% or 95% sequence identity with the particular CDR or set of CDRs listed in the corresponding SEQ ID NO or table hereinafter.

The sequences of the CDRs, according to IMGT, Kabat and Chothia definitions systems, are summarized in table 6 below.

TABLE 6

| | CDR | | | | | | |
|---|---|---|---|---|---|---|---|
| | defi- | SEQ | | SEQ | | SEQ | |
| mAb | nition | ID | Sequence | ID | Sequence | ID | Sequence |
| NKp46-1 | | HCDR1 | | HCDR2 | | HCDR3 | |
| | Kabat | 47 | DYVIN | 50 | EIYPGSGT | 53 | RGRYGLYA |
| | | | | | NYYNEKFK | | MDY |
| | | | | | A | | |
| | Chothia | 48 | GYTFSDY | 51 | YPGSGT | 54 | RGRYGLYA |
| | | | | | | | MDY |
| | IMGT | 49 | GYTFSDYV | 52 | IYPGSGTN | 55 | ARRGRYGL |
| | | | | | | | YAMDY |
| | | LCDR1 | | LCDR2 | | LCDR3 | |
| | Kabat | 56 | RASQDISN | 59 | YTSRLHS | 62 | QQGNTRPW |
| | | | YLN | | | | T |
| | Chothia | 57 | RASQDISN | 60 | YTSRLHS | 63 | QQGNTRPW |
| | | | YLN | | | | T |
| | IMGT | 58 | QDISNY | 61 | YTS | 64 | QQGNTRPW |
| | | | | | | | T |

### IL2 Moiety

In some embodiment, the cytokine moiety of the binding protein of the disclosure is a variant interleukin-2 polypeptide.

The cytokine moiety can be a fragment comprising at least 20, 30, 40, 50, 60, 70, 80 or 100 contiguous amino acids of a human interleukin-2 polypeptide. In certain embodiments, the IL-2 polypeptide is a variant of a human cytokine comprising one or more amino acid modifications (e.g. amino acid substitutions) compared to the wild-type IL-2, for example to decrease binding affinity to a receptor present on non-NK cells, for example Treg cells, CD4 T cells, CD8 T cells.

Optionally, signaling is assessed by bringing the IL-2 (e.g. as a recombinant protein domain or within a multispecific protein of the disclosure) into contact with an NK cell and measuring signaling, e.g. measuring STAT phosphorylation in the NK cells.

In one embodiment, the IL-2 or CD122-specific ABD binds its receptor, as determined by SPR, with a binding affinity (KD) between about 1 nm and about 200 nm, optionally between about 1 nm and about 100 nm optionally between about 10 nM and about 200 nM, optionally between about 10 nM and about 100 nM optionally between about 15 nM and about 100 nM.

The CD122-binding ABD is advantageously a variant or modified IL-2 polypeptide that has reduced binding to CD25 (IL-2Rα) (e.g. reduced or abolished binding affinity, for example as determined by SPR) compared to a wild-type human interleukin-2. Such a variant or modified IL-2 polypeptide is also referred herein to as an “IL2v” or a “not-alpha IL-2”. The CD122-binding ABD can optionally be specified to have a binding affinity for human CD122 that is substantially equivalent to that of wild-type human IL-2. The CD122-binding ABD can optionally be specified to have an ability to induce CD122 signaling and/or binding affinity for CD122 that is substantially equivalent to that of wild-type human IL-2. In one embodiment, the CD122-binding ABD has a reduction in binding affinity for CD25 that is greater than the reduction in binding affinity for CD122, for example a reduction of at least 1-log, 2-log or 3-log in binding affinity for CD25 and a reduction in binding affinity for CD122 that is less than 1-log.

IL-2 is believed to bind IL-2Rβ (CD122) in its form as a monomeric IL-2 receptor (IL-2R), followed by recruitment of the IL-2Rγ (CD132; also termed common γ chain) subunit. In cells that do not express CD25 at their surface, binding (e.g. reduced binding) to CD122 can therefore optionally be specified as being binding in or to a CD122:CD132 complex. The CD122 (or CD122:CD132 complex) can optionally be specified as being present at the surface of an NK cell. In cells that express CD25 at their surface, IL-2 is believed to bind CD25 (IL-2Rα) in its form as a monomeric IL-2 receptor, followed by association of the subunits IL-2Rβ and IL-2Rγ. Binding (e.g. reduced binding, partially reduced binding) to CD25 can therefore optionally be specified as being binding in or to a CD25:CD122 complex or a CD25:CD122:CD132 complex.

In a multispecific protein herein, the multispecific protein can optionally be specified as being configured and/or in a conformation (or capable of adopting a conformation) in which the CD122 ABD (e.g. IL2v) is capable of binding to CD122 at the surface of a cell (e.g. an NK cell, a CD122+CD25− cell) when the multispecific protein is bound to NKp46 (and optionally further to CD16) at the surface of said cell. Optionally further, the multispecific protein:CD122 complex is capable of binding to CD132 at the surface of said cell.

The CD122 ABD or IL2v can be a modified IL-2 polypeptide, for example a monomeric IL-2 polypeptide modified by introducing one more amino acid substitutions, insertions or deletions that decrease binding to CD25.

In some embodiments, where binding to CD25 is sought to be selectively decreased, a IL-2 polypeptide can be modified by binding or associating it with one or more other additional molecules such as polymers or (poly)peptides that result in a further decrease of or abolished binding to CD25. For example a wild-type or mutated IL-2 polypeptide can be modified or further modified by binding to it another moiety that shields, masks, binds or interacts with CD25-binding site of human IL-2, thereby decreasing binding to CD25. In some examples, molecules such as polymers (e.g. PEG polymers) are conjugated to an IL-2 polypeptide to shield or mask the epitope on IL-2 that is bound by CD25, for example by introduction (e.g. substitution) to install an amino acid containing a dedicated chemical hook at a specific site on the IL-2 polypeptide. In other examples, a wild-type or variant IL-2 polypeptide is bound to anti-IL-2 monoclonal antibody or antibody fragment that binds or interacts with CD25-binding site of human IL-2, thereby decreasing binding to CD25.

In any embodiment, an IL2 polypeptide can be a full-length IL-2 polypeptide or it can be an IL-2 polypeptide fragment, so long as the fragment or IL2v that comprises it retains the specified activity (e.g. retaining at least partial CD122 binding, compared to wild-type IL-2 polypeptide).

As shown herein, an IL2v polypeptide can advantageously comprise an IL-2 polypeptide comprising one or more amino acid mutations designed to reduce its ability to bind to human CD25 (IL-2Rα), while retaining at least at least some, or optionally substantially full, ability to bind human CD122.

Various IL2v or not-alpha IL-2 moieties have been described which reduce the activation bias of IL-2 on CD25+ cells. Such IL2v reduce binding to IL-2Rα and maintain at least partial binding to IL-2Rβ. Several IL2v polypeptides have been described, many having mutations in amino acid residue regions 35-72 and/or 79-92 of the IL-2 polypeptide. For example, decreased affinity to IL-2Rα may be obtained by substituting one or more of the following residues in the sequence of a wild-type IL-2 polypeptide: R38, F42, K43, Y45, E62, P65, E68, V69, and L72 (amino acid residue numbering is with reference to the mature IL-2 polypeptide shown in SEQ ID NO: 27).

Wild-type mature human IL-2
(SEQ ID NO: 27)
APTSSSTKKTQLQLEHLLLDLQMILNGINNYKNPKLTRMLTFKFYM
PKKATELKHLQCLEEELKPLEEVLNLAQSKNFHLRPRDLISNINVI
VLELKGSETTFMCEYADETATIVEFLNRWITFCQSIISTLT.

“IL-2p” wild-type mature IL-2 with optional deletion of the three N-terminal residues APA:

(SEQ ID NO: 28)
SSSTKKTQLQLEHLLLDLQMILNGINNYKNPKLTRMLTFKFYMPKK
ATELKHLQCLEEELKPLEEVLNLAQSKNFHLRPRDLISNINVIVLE
LKGSETTFMCEYADETATIVEFLNRWITFCQSIISTLT.

An exemplary IL2v (also referred to herein as IL2v in the Examples) can have the amino acid of wild-type IL-2 with the five amino acid substitutions T3A, F42A, Y45A, L72G and C125A, as shown below, optionally further with deletion of the three N-terminal residues APA:

(SEQ ID NO: 24)
APASSSTKKTQLQLEHLLLDLQMILNGINNYKNPKLTRMLTAKFAM
PKKATELKHLQCLEEELKPLEEVLNGAQSKNFHLRPRDLISNINVI
VLELKGSETTFMCEYADETATIVEFLNRWITFAQSIISTLT.

As few as one or two mutations can reduce binding to IL-2Rα and L-2Rβ. For example, as exemplified in the multispecific proteins herein, the IL2v polypeptide having two amino acid substitutions R38A and F42K in the wild-type human IL-2 amino acid sequence displayed suitable reduced binding to IL-2Rα, with retention of binding to IL-2Rβ resulting in highly active multispecific proteins, referred to herein as IL2v2.

IL2v2 (R38A/F42K substitutions):
(SEQ ID NO: 25)
SSSTKKTQLQLEHLLLDLQMILNGINNYKNPKLTAMLTKKFYMPKK
ATELKHLQCLEEELKPLEEVLNLAQSKNFHLRPRDLISNINVIVLE
LKGSETTFMCEYADETATIVEFLNRWITFCQSIISTLT.

In one embodiment, an IL2v2 polypeptide can further encompass substitution C125A (with reference to the wild-type mature human IL-2 of SEQ ID NO: 27), referred to herein as IL2v2A.

IL2V2A (R38A/F42K/C125A substitutions):
(SEQ ID NO: 65)
SSSTKKTQLQLEHLLLDLQMILNGINNYKNPKLTAMLTKKFYMPKK
ATELKHLQCLEEELKPLEEVLNLAQSKNFHLRPRDLISNINVIVLE
LKGSETTFMCEYADETATIVEFLNRWITFAQSIISTLT.

In one embodiment, an IL2v polypeptide has the wild-type IL-2p amino acid sequence with the three amino acid substitutions R38A, F42K and T41A (with reference to the wild-type mature human IL-2 of SEQ ID NO: 27), as shown below, referred to herein as IL2v3:

IL2v3 (R38A/T41A/F42K substitutions):
(SEQ ID NO: 26)
SSSTKKTQLQLEHLLLDLQMILNGINNYKNPKLTAMLAKKFYMPKK
ATELKHLQCLEEELKPLEEVLNLAQSKNFHLRPRDLISNINVIVLE
LKGSETTFMCEYADETATIVEFLNRWITFCQSIISTLT.

Thus, in one embodiment, an IL2 variant comprises at least one or at least two amino acid modifications (e.g. substitution, insertion, deletion) compared to a human wild type IL-2 polypeptide. In one embodiment, an IL2v comprises a R38 substitution (e.g. R38A) and an F42 substitution (e.g., F42K), compared to a human wild type IL-2 polypeptide. In one embodiment, an IL2v comprises a R38 substitution (e.g. R38A), an F42 substitution (e.g., F42K) and a T41 substitution (e.g. T41A), compared to a human wild type IL-2 polypeptide. In one embodiment, an IL2v comprises a T3 substitution (e.g. T3A), an F42 substitution (e.g., F42A), a Y45 substitution (e.g. Y45A), a L72 substitution (e.g. L72G) and a C125 substitution (e.g. C125A), compared to a human wild type IL-2 polypeptide. Optionally the IL2v comprises an amino acid sequence identical to or at least 70%, 80%, 90%, 95%, 98% or 99% identical to the polypeptide of SEQ ID NOS: 24-26 or 65. Optionally the IL2v comprises a fragment of a human IL-2 polypeptide, wherein the fragment has an amino sequence identical to or at least 70%, 80%, 90%, 95%, 98% or 99% identical to a contiguous sequence of 40, 50, 60, 70 or 80 amino acids of the polypeptide of SEQ ID NOS: 24-26 or 65.

Any combination of the positions can be modified. In some embodiments, the IL-2 variant comprises two or more modification. In some embodiments, the IL-2 variant comprises three or more modification. In some embodiments, the IL-2 variant comprises four, five, or six or more modifications.

IL2 variant polypeptides can for example comprise two, three, four, five, six or seven amino acid modifications (e.g. substitutions). For example, U.S. Pat. No. 5,229,109, the disclosure of which is incorporated herein by reference, provides a human IL2 polypeptide having a R38A and F42K substitution. U.S. Pat. No. 9,447,159, the disclosure of which is incorporated herein by reference, describes human IL2 polypeptides having substitutions T3A, F42A, Y45A, and L72G substitutions. U.S. Pat. No. 9,266,938, the disclosure of which is incorporated herein by reference, describes human IL2 polypeptides having substitutions at residue L72 (e.g. L72G, L72A, L72S, L72T, L72Q, L72E, L72N, L72D, L72R, and L72K), residue F42 (e.g. F42A, F42G, F42S, F42T, F42Q, F42E, F42N, F42D, F42R, and F42K); and at residue Y45 (e.g., Y45A, Y45G, Y45S, Y45T, Y45Q, Y45E, Y45N, Y45D, Y45R and Y45K), including for example the triple mutation F42A/Y45A/L72G to reduce or abolish the affinity for IL-2Rα receptor. Yet further WO2020/057646, the disclosure of which is incorporated herein by reference, relates to amino acid sequence of IL-2v polypeptides comprising amino acid substitutions in various combinations among amino acid residues K35, T37, R38, F42, Y45, E61 and E68. Yet further, WO2020252418, the disclosure of which is incorporated herein by reference, relates to amino acid sequence of IL-2v polypeptides having at least one amino acid residues position R38, T41, F42, F44, E62, P65, E68, Y107, or C125 substituted with another amino acid, for example wherein the amino acid substitution is selected from the group consisting of: the substitution of L19D, L19H, L19N, L19P, L19Q, L19R, L19S, L19Y at position 19, the substitution of R38A, R38F, R38G at position 38, the substitution of T41A, T41G, and T41V at position 41, the substitution of F42A at position 42, the substitution of F44G and F44V at position 44, the substitution of E62A, E62F, E62H, and E62L at position 62, the substitution of P65A, P65E, P65G, P65H, P65K, P65N, P65Q, P65R at position 65, the substitution of E68E, E68F, E68H, E68L, and E68P at position 68, the substitution of Y107G, Y107H, Y107L and Y107V at position 107, and the substitution of C1251 at position 125, the substitution of Q126E at position 126. Numbering of positions is with respect to Wild-type mature human IL-2.

A modified IL-2 can have a lower binding affinity for its receptor(s), optionally the modified IL-2 can be specified as exhibiting a KD for binding to CD25 or to a CD25:CD122:CD132 complex that is within 1-log, optionally 2-log, optionally 3-log, of the KD of a wild-type human IL-2 polypeptide (e.g. comprising the amino acid sequence of SEQ ID NO: 27). A modified IL-2 can optionally be specified as exhibiting less than 20%, 30%, 40% or 50% of binding affinity to CD25 or to a CD25:CD122:CD132 complex compared to a wild-type human IL-2 polypeptide. An IL2 can optionally be specified as exhibiting at least 50%, 70%, 80% or 90% of binding affinity to CD122 or to a CD122:CD132 complex compared to a wild-type human IL-2 polypeptide. In some embodiments, an IL2 exhibits at least 50%, 60%, 70% or 80% but less than 100% of binding affinity to CD122 or to a CD122:CD132 complex compared to a wild-type human IL-2 polypeptide. In some embodiments, an IL2v exhibits less than 50% of binding affinity to CD25 and at least 50%, 60%, 70% or 80% of binding affinity to CD122, compared to wild-type IL-2 polypeptide.

Differences in binding affinity of wild-type and disclosed mutant polypeptide for CD25 and CD122 and complexes thereof can be measured, e.g., in standard surface plasmon resonance (SPR) assays that measure affinity of protein-protein interactions familiar to those skilled in the art.

Exemplary IL2 variant polypeptides have one or more, two or more, or three or more CD25-affinity-reducing amino acid substitutions relative to the wild-type mature IL-2 polypeptide having an amino acid sequence of SEQ ID NO: 27. In one embodiment, the exemplary IL2v polypeptides comprise one or more, two or more, or three or more substituted residues selected from the following group: Q11, H16, L18, L19, D20, D84, S87, Q22, R38, T41, F42, K43, Y45, E62, P65, E68, V69, L72, D84, S87, N88, V91, 192, T123, Q126, S127, 1129, and S130.

In one embodiment, the exemplary IL2 variant polypeptide has one, two, three, four, five or more of amino acid residues position R38, T41, F42, F44, E62, P65, E68, Y107, or C125 substituted with another amino acid.

In one embodiment, decreased affinity to CD25 or a protein complex comprising such (e.g., a CD25:CD122:CD132 complex) may be obtained by substituting one or more of the following residues in the sequence of the wild-type mature IL-2 polypeptide: R38, F42, K43, Y45, E62, P65, E68, V69, and L72.

In yet other examples, an IL-2 polypeptide is modified by connecting, fusing, binding or associating it with one or more other additional compounds, chemical compounds, polymer (e.g. PEG), or polypeptides or polypeptide chains that result in a decrease of binding to CD25. For example a wild-type IL-2 polypeptide or fragment thereof can be modified by binding to it a CD25 binding peptide or polypeptide, including but not limited to an anti-IL-2 monoclonal antibody or antibody fragment thereof that binds or interacts with CD25-binding site of human IL-2, thereby decreasing binding to CD25.

In other examples, an IL-2 polypeptide or fragment thereof can be modified by binding to it a moiety of interest (e.g. a compound, chemical compounds, polymer, linear or branched PEG polymer), covalently attached to a natural amino acid or to an unnatural amino acid installed at a selected position. Such a modified interleukin 2 (IL-2) polypeptide can comprise at least one unnatural amino acid at a position on the polypeptide that reduces binding between the modified IL-2 polypeptide and CD25 but retains significant binding to the CD122:CD132 signaling complex, wherein the reduced binding to CD25 is compared to binding between a wild-type IL-2 polypeptide and CD25. An unnatural amino acid can be positioned at any one or more of residues K35, T37, R38, T41, F42, K43, F44, Y45, E60, E61, E62, K64, P65, E68, V69, N71, L72, M104, C105, and Y107 of IL-2. As disclosed in PCT publication nos. WO2019/028419 and WO2019/014267, the disclosures of which are incorporated herein by reference, the unnatural amino acid can be incorporated into the modified IL-2 polypeptide by an orthogonal tRNA synthetase/tRNA pair. The unnatural amino acid can for example comprise a lysine analogue, an aromatic side chain, an azido group, an alkyne group, or an aldehyde or ketone group. The modified IL-2 polypeptide can then be covalently attached to a water-soluble polymer, a lipid, a protein, or a peptide through the unnatural amino acid. Examples of suitable polymers include polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(a-hydroxy acid), poly(vinyl alcohol), polyphosphazene, polyoxazolines (POZ), poly(N-acryloylmorpholine), or a combination thereof, or a polysaccharide such as dextran, polysialic acid (PSA), hyaluronic acid (HA), amylose, heparin, heparan sulfate (HS), dextrin, or hydroxyethyl-starch (HES).

### Constant Domain

Constant region domains can be derived from any suitable human antibody, particularly human antibodies of gamma isotype, including, the constant heavy (CH1) and light (CL, Cκ or Cλ) domains, hinge domains, CH2 and CH3 domains.

With respect to heavy chain constant domains, “CH1” generally refers to positions 118-220 according to the EU index as in Kabat. Depending on the context, a CH1 domain (e.g. as shown in the domain arrangements), can optionally comprise residues that extend into the hinge region such that the CH1 comprises at least part of a hinge region. For example, when positioned C-terminal on a polypeptide chain and/or or C-terminal to the Fc domain, and/or within a Fab structure that is or C-terminal to the Fc domain, the CH1 domain can optionally comprise at least part of a hinge region, for example CH1 domains can comprise at least an upper hinge region, for example an upper hinge region of a human IgG1 hinge, optionally further in which the terminal threonine of the upper hinge can be replaced by a serine. Such a CH2 domain can therefore comprise at its C-terminus the amino acid sequence: EPKSCDKTHS.

Exemplary human CH1 domain amino acid sequences includes:

(SEQ ID NO: 12)
ASTKGPSVFPLAPSSKSTSGGTAALGCLVKDYFPEPVTVSWNSGAL
TSGVHTFPAVLQSSGLYSLSSVVTVPSSSLGTQTYICNVNHKPSNT
KVDKRV.

Exemplary human Cκ domain amino acid sequences include:

(SEQ ID NO: 4)
RTVAAPSVFIFPPSDEQLKSGTASVVCLLNNFYPREAKVQWKVDNA
LQSGNSQESVTEQDSKDSTYSLSSTLTLSKADYEKHKVYACEVTHQ
GLSSPVTKSFNRGEC.

In some exemplary configurations, the multispecific protein can be a heterodimer or a heterotrimer comprising one or two Fabs (e.g. one Fab binding NKp46 and the other binding CD20), in which variable regions, CH1 and/or CL domains are engineered by introducing amino acid substitutions in a knob-into-holes or electrostatic steering approach to promote the desired chain pairings of CH1 domains with CK domains. In some exemplary configurations, the multispecific protein can be a heterodimer, or a heterotrimer comprising one or two Fabs (e.g. one Fab binding NKp46 and the other binding CD20), wherein a Fab has a VH/VL crossover (VH and VL replace one another) or a CH1/CL crossover (the CH1 and CL replace one another), and wherein the CH1 and/or CL domains comprise amino acid substitutions to promote correct chain association by knob-into-holes or electrostatic steering.

“CH2” generally refers to positions 237-340 according to the EU index as in Kabat, and “CH3” generally refers to positions 341-447 according to the EU index as in Kabat. CH2 and CH3 domains can be derived from any suitable antibody. Such CH2 and CH3 domains can be used as wild-type domains or may serve as the basis for a modified CH2 or CH3 domain. Optionally the CH2 and/or CH3 domain is of human origin or may comprise that of another species (e.g., rodent, rabbit, non-human primate) or may comprise a modified or chimeric CH2 and/or CH3 domain, e.g., one comprising portions or residues from different CH2 or CH3 domains, e.g., from different antibody isotypes or species antibodies.

Exemplary human I G1 CH2 domain amino acid sequence includes:

(SEQ ID NO: 7)
APELLGGPSVFLFPPKPKDTLMISRTPEVTCVVVDVSHEDPEVKFN
WYVDGVEVHNAKTKPREEQYNSTYRVVSVLTVLHQDWLNGKEYKCK
VSNKALPAPIEKTISKAK.

Exemplary human IgG1 CH3 domain amino acid sequence includes:

(SEQ ID NO: 8)
GQPREPQVYTLPPSREEMTKNQVSLTCLVKGFYPSDIAVEWESNGQ
PENNYKTTPPVLDSDGSFFLYSKLTVDKSRWQQGNVFSCSVMHEAL
HNHYTQKSLSLSPGK.

In any of the domain arrangements, the Fc domain monomer may comprise a CH2-CH3 unit (a full length CH2 and CH3 domain or a fragment thereof). In heterodimers or heterotrimers comprising two chains with Fc domain monomers (i.e. the heterodimers or heterotrimers comprise a Fc domain dimer), the CH3 domain will be capable of CH3-CH3 dimerization (e.g. it will comprise a wild-type CH3 domain or a CH3 domain with modifications to promote a desired CH3-CH3 dimerization). An Fc domain may optionally further comprise a C-terminal lysine (K) (See SEQ ID NO: 6).

Exemplary human IgG1 CH2-CH3 (Fc) domain amino acid sequences include:

(SEQ ID NO: 6)
APELLGGPSVFLFPPKPKDTLMISRTPEVTCVVVDVSHEDPEVKFN
WYVDGVEVHNAKTKPREEQYNSTYRVVSVLTVLHQDWLNGKEYKCK
VSNKALPAPIEKTISKAKGQPREPQVYTLPPSREEMTKNQVSLTCL
VKGFYPSDIAVEWESNGQPENNYKTTPPVLDSDGSFFLYSKLTVDK
SRWQQGNVFSCSVMHEALHNHYTQKSLSLSPGK.
or
(SEQ ID NO: 14)
APELLGGPSVFLFPPKPKDTLMISRTPEVTCVVVDVSHEDPEVKFN
WYVDGVEVHNAKTKPREEQYNSTYRVVSVLTVLHQDWLNGKEYKCK
VSNKALPAPIEKTISKAKGQPREPQVYTLPPSREEMTKNQVSLTCL
VKGFYPSDIAVEWESNGQPENNYKTTPPVLDSDGSFFLYSKLTVDK
SRWQQGNVFSCSVMHEALHNHYTQKSLSLSPG.

In some exemplary configurations, the multispecific protein can be a heterodimer, a heterotrimer or a heterotetramer, wherein the polypeptide chains are engineered for heterodimerization among each other so as to produce the desired protein. In embodiments where the desired chain pairings are not driven by CH1-Cκ dimerization or where enhancement of pairing is desired, the chains may comprise constant or Fc domains with amino acid modifications (e.g., substitutions) that favor the preferential hetero-dimerization of the two different chains over the homo-dimerization of two identical chains.

In some embodiments, a “knob-into-holes” approach is used in which the domain interfaces (e.g. CH3 domain interface of the antibody Fc region) are mutated so that the antibodies preferentially heterodimerize. These mutations create altered charge polarity across the interface (e.g. Fc dimer interface) such that co-expression of electrostatically matched chains (e.g. Fc-containing chains) support favorable attractive interactions thereby promoting desired heterodimer (e.g. Fc heterodimer) formation, whereas unfavorable repulsive charge interactions suppress unwanted heterodimer (e.g., Fc homodimer) formation. See for example mutations and approaches reviewed in Brinkmann and Kontermann, 2017 MAbs, 9(2): 182-212, the disclosure of which is incorporated herein by reference. For example one heavy chain comprises a T366W substitution and the second heavy chain comprises a T366S, L368A and Y407V substitution, see, e.g. Ridgway et al (1996) Protein Eng., 9, pp. 617-621; Atwell (1997) J. Mol. Biol., 270, pp. 26-35; and WO2009/089004, the disclosures of which are incorporated herein by reference. In another approach, one heavy chain comprises a F405L substitution and the second heavy chain comprises a K409R substitution, see, e.g., Labrijn et al. (2013) Proc. Natl. Acad. Sci. U.S.A., 110, pp. 5145-5150. In another approach, one heavy chain comprises T350V, L351Y, F405A, and Y407V substitutions and the second heavy chain comprises T350V, T366S, K392L, and T394W substitutions, see, e.g. Von Kreudenstein et al., (2013) mAbs 5:646-654. In another approach, one heavy chain comprises both K409D and K392D substitutions and the second heavy chain comprises both D399K and E356K substitutions, see, e.g. Gunasekaran et al., (2010) J. Biol. Chem. 285:19637-19646. In another approach, one heavy chain comprises D221E, P228E and L368E substitutions and the second heavy chain comprises D221R, P228R, and K409R substitutions, see, e.g. Strop et al., (2012) J. Mol. Biol. 420: 204-219. In another approach, one heavy chain comprises S364H and F405A substitutions and the second heavy chain comprises Y349T and, T394F substitutions, see, e.g. Moore et al., (2011) mAbs 3: 546-557. In another approach, one heavy chain comprises a H435R substitution and the second heavy chain optionally may or may not comprise a substitution, see, e.g. U.S. Pat. No. 8,586,713. When such hetero-multimeric antibodies have Fc regions derived from a human IgG2 or IgG4, the Fc regions of these antibodies can be engineered to contain amino acid modifications that permit CD16 binding. In some embodiments, the antibody may comprise mammalian antibody-type N-linked glycosylation at residue N297 (Kabat EU numbering).

In some embodiments, one or more pairs of disulfide bonds such as A287C and L306C, V259C and L306C, R292C and V302C, and V323C and I332C are introduced into the Fc region to increase stability, for example further to a loss of stability caused by other Fc modifications. Additional example includes introducing K3381, A339K, and K340S mutations to enhance Fc stability and aggregation resistance (Gao et al, 2019 Mol Pharm. 2019; 16:3647).

In some embodiments, where a multispecific protein is intended to have reduced binding to a human Fc gamma receptor. In some embodiments, where a multispecific protein is intended to have reduced binding to a human CD16A polypeptide (and optionally further reduced binding to CD32A, CD32B and/or CD64), the Fc domain is a human IgG4 Fc domain, optionally further wherein the Fc domain comprises a S228P mutation to stabilize the hinge disulfide.

In embodiments, where a multispecific protein is intended to have reduced binding to human CD16A polypeptide (and optionally further reduced binding to CD32A, CD32B and/or CD64), a CH2 and/or CH3 domain (or Fc domain comprising same) may comprise a modification to decrease or abolish binding to FcγRIIIA (CD16). For example, CH2 mutations in a Fc domain dimer proteins at reside N297 (Kabat numbering) can substantially eliminate CD16A binding. However the person of skill in the art will appreciate that other configurations can be implemented. For example, substitutions into human IgG1 or IgG2 residues at positions 234-237 and/or residues at positions 327, 330 and 331 were shown to greatly reduce binding to Fcγ receptors and thus ADCC and CDC. Furthermore, Idusogie et al. (2000) J. Immunol. 164(8):4178-84 demonstrated that alanine substitution at different positions, including K322, significantly reduced complement activation.

In one embodiment, the asparagine (N) at Kabat heavy chain residue 297 can be substituted by a residue other than an asparagine (e.g. a serine).

In one embodiment, an Fc domain modified to reduce binding to CD16A comprises a substitution in the Fc domain at Kabat residues 234, 235, 237, 330 and 331. In one embodiment, the Fc domain is of human IgG1 subtype. Amino acid residues are indicated according to EU numbering according to Kabat.

In one embodiment, an Fc domain modified to reduce binding to CD16A comprises an amino acid modification (e.g. substitution) at one or more of Kabat residue(s) 233-237, and an amino acid modification (e.g. substitution) at Kabat residue(s) 330 and/or 331. One example of such an Fc domain comprises substitutions at Kabat residues L234, L235, G237, A330 and P331 (e.g., L234A/L235E/G237A/A330S/P331S).

In one embodiment, an Fc domain that has low or reduced binding to CD16A comprises a human IgG1 Fc domain, wherein the CH2-CH3 domain has the amino acid sequence below (human IgG1 with N297S substitution), or an amino acid sequence at least 90%, 95% or 99% identical thereto.

(SEQ ID NO: 89)
APELLGGPSVFLFPPKPKDTLMISRTPEVTCVVVDVSHEDPEVKFN
WYVDGVEVHNAKTKPREEQYSSTYRVVSVLTVLHQDWLNGKEYKCK
VSNKALPAPIEKTISKAKGQPREPQVYTLPPSREEMTKNQVSLTCL
VKGFYPSDIAVEWESNGQPENNYKTTPPVLDSDGSFFLYSKLTVDK
SRWQQGNVFSCSVMHEALHNHYTQKSLSLSPG.

In one embodiment, an Fc domain modified to reduce binding to CD16A comprises a CH2-CH3 domain with the amino acid sequence below, or an amino acid sequence at least 90%, 95% or 99% identical thereto but retaining the amino acid residues at Kabat positions 234, 235, 237, 330 and 331 (underlined):

(SEQ ID NO: 90)
APEAEGAPSVFLFPPKPKDTLMISRTPEVTCVVVDVSHEDPEVKFN
WYVDGVEVHNAKTKPREEQYNSTYRVVSVLTVLHQDWLNGKEYKCK
VSNKALPSSIEKTISKAKGQPREPQVYTLPPSREEMTKNQVSLTCL
VKGFYPSDIAVEWESNGQPENNYKTTPPVLDSDGSFFLYSKLTVDK
SRWQQGNVFSCSVMHEALHNHYTQKSLSLSPG.

Any of the above Fc domain sequences can optionally further comprise a C-terminal lysine (K), i.e. as in the naturally occurring sequence.

In certain embodiments herein where binding to CD16 (CD16A) is desired, a CH2 and/or CH3 domain (or Fc domain comprising same) may be a wild-type domain or may comprise one or more amino acid modifications (e.g. amino acid substitutions) which increase binding to human CD16 and optionally another receptor such as FcRn. Optionally, the modifications will not substantially decrease or abolish the ability of the Fc-derived polypeptide to bind to neonatal Fc receptor (FcRn), e.g. human FcRn. Typical modifications include modified human IgG1-derived constant regions comprising at least one amino acid modification (e.g. substitution, deletions, insertions), and/or altered types of glycosylation, e.g., hypofucosylation. Such modifications can affect interaction with Fc receptors: FcγRI (CD64), FcγRII (CD32), and FcγRIII (CD16). FcγRI (CD64), FcγRIIA (CD32A) and FcγRIII (CD 16) are activating (i.e., immune system enhancing) receptors while FcγRIIB (CD32B) is an inhibiting (i.e., immune system dampening) receptor. A modification may, for example, increase binding of the Fc domain to FcγRIIIa on effector (e.g. NK) cells and/or decrease binding to FcγRIIB. Examples of modifications are provided in PCT publication no. WO2014/044686, the disclosure of which is incorporated herein by reference. Specific mutations (in IgG1 Fc domains) which affect (enhance) FcγRIIIa or FcRn binding are also set forth below.

TABLE 7

| | | | | Effect of |
|---|---|---|---|---|
| Isotype | Species | Modification | Effector Function | Modification |
| IgG1 | Human | T250Q/M428L | Increased binding to | Increased half- |
| | | | FcRn | life |
| IgG1 | Human | 1M252Y/S254T/T256E + | Increased binding to | Increased half- |
| | | H433K/N434F | FcRn | life |
| IgG1 | Human | E333A | Increased binding to | Increased ADCC |
| | | | FcγRIIIa | and CDC |
| IgG1 | Human | S239D/I332E or | Increased binding to | Increased ADCC |
| | | S239D/A330L/I332E | FcγRIIIa | |
| IgG1 | Human | P257I/Q311 | Increased binding to | Unchanged half- |
| | | | FcRn | life |
| IgG1 | Human | S239D/I332E/G236A | Increased | Increased |
| | | | FcγRIIa/FcγRIIb | macrophage |
| | | | ratio | phagocytosis |

In some embodiments, the multispecific protein comprises a variant Fc region comprise at least one amino acid modification (for example, possessing 1, 2, 3, 4, 5, 6, 7, 8, 9, or more amino acid modifications) in the CH2 and/or CH3 domain of the Fc region, wherein the modification enhances binding to a human CD16 polypeptide. In other embodiments, the multispecific protein comprises at least one amino acid modification (for example, 1, 2, 3, 4, 5, 6, 7, 8, 9, or more amino acid modifications) in the CH2 domain of the Fc region from amino acids 237-341, or within the lower hinge-CH2 region that comprises residues 231-341. In some embodiments, the multispecific protein comprises at least two amino acid modifications (for example, 2, 3, 4, 5, 6, 7, 8, 9, or more amino acid modifications), wherein at least one of such modifications is within the CH3 region and at least one such modifications is within the CH2 region. Encompassed also are amino acid modifications in the hinge region. In one embodiment, encompassed are amino acid modifications in the CH1 domain, optionally in the upper hinge region that comprises residues 216-230 (Kabat EU numbering). Any suitable functional combination of Fc modifications can be made, for example any combination of the different Fc modifications which are disclosed in any of U.S. Pat. Nos. 7,632,497; 7,521,542; 7,425,619; 7,416,727; 7,371,826; 7,355,008; 7,335,742; 7,332,581; 7,183,387; 7,122,637; 6,821,505 and 6,737,056; and/or in PCT Publications Nos. WO2011/109400; WO 2008/105886; WO 2008/002933; WO 2007/021841; WO 2007/106707; WO 06/088494; WO 05/115452; WO 05/110474; WO 04/1032269; WO 00/42072; WO 06/088494; WO 07/024249; WO 05/047327; WO 04/099249 and WO 04/063351; and/or in Lazar et al. (2006) Proc. Nat. Acad. Sci. USA 103(11): 405-410; Presta, L. G. et al. (2002) Biochem. Soc. Trans. 30(4):487-490; Shields, R. L. et al. (2002) J. Biol. Chem. 26; 277(30):26733-26740 and Shields, R. L. et al. (2001) J. Biol. Chem. 276(9):6591-6604).

In some embodiments, the multispecific protein comprises an Fc domain comprising at least one amino acid modification (for example, 1, 2, 3, 4, 5, 6, 7, 8, 9, or more amino acid modifications) relative to a wild-type Fc region, such that the molecule has an enhanced binding affinity for human CD16 relative to the same molecule comprising a wild-type Fc region, optionally wherein the variant Fc region comprises a substitution at any one or more of positions 221, 239, 243, 247, 255, 256, 258, 267, 268, 269, 270, 272, 276, 278, 280, 283, 285, 286, 289, 290, 292, 293, 294, 295, 296, 298, 300, 301, 303, 305, 307, 308, 309, 310, 311, 312, 316, 320, 322, 326, 329, 330, 332, 331, 332, 333, 334, 335, 337, 338, 339, 340, 359, 360, 370, 373, 376, 378, 392, 396, 399, 402, 404,416, 419, 421, 430,434, 435, 437, 438 and/or 439 (Kabat EU numbering).

In one embodiment, the multispecific protein comprises an Fc domain comprising at least one amino acid modification (for example, 1, 2, 3, 4, 5, 6, 7, 8, 9, or more amino acid modifications) relative to a wild-type Fc region, such that the molecule has enhanced binding affinity for human CD16 relative to a molecule comprising a wild-type Fc region, optionally wherein the variant Fc region comprises a substitution at any one or more of positions 239, 298, 330, 332, 333 and/or 334 (e.g. S239D, S298A, A330L, 1332E, E333A and/or K334A substitutions), optionally wherein the variant Fc region comprises a substitution at residues S239 and 1332, e.g. a S239D and 1332E substitution (Kabat EU numbering).

In some embodiments, the multispecific protein comprises an Fc domain comprising N-linked glycosylation at Kabat residue N297. In some embodiments, the multispecific protein comprises an Fc domain comprising altered glycosylation patterns that increase binding affinity for human CD16. Such carbohydrate modifications can be accomplished by, for example, by expressing a nucleic acid encoding the multispecific protein in a host cell with altered glycosylation machinery. Cells with altered glycosylation machinery are known in the art and can be used as host cells in which to express recombinant antibodies to thereby produce an antibody with altered glycosylation. See, for example, Shields, R. L. et al. (2002) J. Biol. Chem. 277:26733-26740; Umana et al. (1999) Nat. Biotech. 17:176-1, as well as, European Patent No: EP 1176195; PCT Publications WO 06/133148; WO 03/035835; WO 99/54342, each of which is incorporated herein by reference in its entirety. In one aspect, the multispecific protein contains one or more hypofucosylated constant regions. Such multispecific protein may comprise an amino acid alteration or may not comprise an amino acid alteration and/or may be expressed or synthesized or treated under conditions that result in hypofucosylation. In one aspect, a multispecific protein composition comprises a multispecific protein described herein, wherein at least 20, 30, 40, 50, 60, 75, 85, 90, 95% or substantially all of the antibody species in the composition have a constant region comprising a core carbohydrate structure (e.g. complex, hybrid and high mannose structures) which lacks fucose. In one embodiment, provided is a multispecific protein composition which is free of N-linked glycans comprising a core carbohydrate structure having fucose. The core carbohydrate will preferably be a sugar chain at Asn297.

Optionally, a multispecific protein comprising a Fc domain dimer can be characterized by having a binding affinity to a human CD16A polypeptide that is within 1-log of that of a conventional human IgG1 antibody, e.g., as assessed by surface plasmon resonance.

In one embodiment, the multispecific protein comprising a Fc domain dimer in which an Fc domain is engineered to enhance Fc receptor binding can be characterized by having a binding affinity to a human CD16A polypeptide that is at least 1-log greater than that of a conventional or wild-type human IgG1 antibody, e.g., as assessed by surface plasmon resonance.

In one embodiment, a multispecific protein comprising a Fc domain dimer can be characterized by having a binding affinity to a human FcRn (neonatal Fc receptor) polypeptide that is within 1-log of that of a conventional human IgG1 antibody, e.g., as assessed by surface plasmon resonance.

Optionally a multispecific protein comprising a Fc domain dimer can be characterized by a Kd for binding (monovalent) to a human Fc receptor polypeptide (e.g., CD16A) of less than 10⁻⁵ M (10 μmolar), optionally less than 10⁻⁶ M (1 μmolar), as assessed by surface plasmon resonance (e.g. as in the Examples herein, SPR measurements performed on a Biacore T100 apparatus (Biacore GE Healthcare), with bispecific antibodies immobilized on a Sensor Chip CM5 and serial dilutions of soluble CD16 polypeptide injected over the immobilized bispecific antibodies.

### Connections and Linkers

Generally, there are a number of suitable linkers that can be used in the multispecific proteins, including traditional peptide bonds, generated by recombinant techniques. In some embodiments, the linker is a “domain linker”, used to link any two domains as outlined herein together. Adjacent protein domains can be specified as being connected or fused to one another by a domain linker. An exemplary domain linker is a (poly)peptide linker, optionally a flexible (poly)peptide linker. Peptide linkers or polypeptide linkers, used interchangeably herein, may have a subsequence derived from a particular domain such as a hinge, CH1 or CL domain, or may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 2 to 30 amino acids in length. In one embodiment, linkers of 4 to 20 amino acids in length may be used, with from about 5 to about 15 amino acids finding use in some embodiments. While any suitable linker can be used, many embodiments, linkers (e.g. flexible linkers) can utilize a glycine-serine polypeptide or polymer, including for example comprising (GS)ₙ, (GSGGS)ₙ, (GGGGS)ₙ, (GSSS)ₙ, (GSSSS)ₙ and (GGGS)ₙ, where n is an integer of at least one (optionally n is 1, 2, 3 or 4), glycine-alanine polypeptide, alanine-serine polypeptide, and other flexible linkers. Linkers comprising glycine and serine residues generally provides protease resistance. One example of a (GS)₁ linker is a linker having the amino acid sequence STGS; such a linker can be useful to fuse a domain to the C-terminus of an Fc domain (or a CH3 domain thereof). In some embodiments peptide linkers comprising (G₂S)ₙ are used, wherein, for example, n=1-20, e.g., (G₂S), (G₂S)₂, (G₂S)₃, (G₂S)₄, (G₂S)₅, (G₂S)₆, (G₂S)₇ or (G₂S)₈, or (G₃S)ₙ, wherein, for example, n is an integer from 1-15. In one embodiment, a domain linker comprises a (G₄S)ₙ peptide, wherein, for example, n is an integer from 1-10, optionally 1-6, optionally 1-4. In some embodiments peptide linkers comprising (GS₂)ₙ (GS₃)ₙ or (GS₄)ₙ are used, wherein, for example, n=1-20, e.g., (GS₂), (GS₂)₂, (GS₂)₃, (GS₃)₁, (GS₃)₂, (GS₃)₃, (GS₄)₁, (GS₄)₂, (GS₄)₃, wherein, for example, n is an integer from 1-15. In one embodiment, a domain linker comprises a (GS₄)ₙ peptide, wherein, for example, n is an integer from 1-10, optionally 1-6, optionally 1-4. In one embodiment, a domain linker comprises a C-terminal GS dipeptide, e.g., the linker comprises (GS₄) and has the amino acid sequence a GSSSS (SEQ ID NO: 20), GSSSSGSSSS (SEQ ID NO: 21), GSSSSGSSSSGS (SEQ ID NO: 22) or GSSSSGSSSSGSSSS (SEQ ID NO: 23).

Any of the peptide or domain linkers may be specified to comprise a length of at least 4 residues, at least 5 residues, at least 10 residues, at least 15 residues, at least 20 residues, or more. In other embodiments, the linkers comprise a length of between 2-4 residues, between 2-4 residues, between 2-6 residues, between 2-8 residues, between 2-10 residues, between 2-12 residues, between 2-14 residues, between 2-16 residues, between 2-18 residues, between 2-20 residues, between 2-22 residues, between 2-24 residues, between 2-26 residues, between 2-28 residues, between 2-30 residues, between 2 and 50 residues, or between 10 and 50 residues.

Examples of polypeptide linkers may include sequence fragments from CH1 or CL domains; for example the first 4-12 or 5-12 amino acid residues of the CL/CH1 domains are particularly useful for use in linkages of scFv moieties. Linkers can be derived from immunoglobulin light chains, for example Cκ or Cλ. Linkers can be derived from immunoglobulin heavy chains of any isotype, including for example Cy1, Cy2, Cy3, Cy4 and Cμ. Linker sequences may also be derived from other proteins such as Ig-like proteins (e.g. TCR, FcR, KIR), hinge region-derived sequences, and other natural sequences from other proteins. In certain domain arrangements, V_{H} and V_{L} domains are linked to another in tandem separated by a linker peptide (e.g. an scFv) and in turn be fused to the N- or C-terminus of an Fc domain (or CH2 domain thereof). Such tandem variable regions or scFv can be connected to the Fc domain via a hinge region or a portion thereof, an N-terminal fragment of a CH1 or CL domain, or a glycine- and serine-containing flexible polypeptide linker.

Fc domains can be connected to other domains via immunoglobulin-derived sequence or via non-immunoglobulin sequences, including any suitable linking amino acid sequence. Advantageously, immunoglobulin-derived sequences can be readily used between CH1 or CL domains and Fc domains, in particular, where a CH1 or CL domain is fused at its C-terminus to the N-terminus of an Fc domain (or CH2 domain). An immunoglobulin hinge region or portion of a hinge region can and generally will be present on a polypeptide chain between a CH1 domain and a CH2 domain. A hinge or portion thereof can also be placed on a polypeptide chain between a CL (e.g. Cκ) domain and the CH2 domain of an Fc domain when a CL is adjacent to an Fc domain on the polypeptide chain. However, it will be appreciated that a hinge region can optionally be replaced for example by a suitable linker peptide, e.g. a flexible polypeptide linker.

The NKp46 ABD and CD122 ABD (e.g., a cytokine) are advantageously linked to the rest of the multispecific protein (e.g. or to a constant domain or Fc domain thereof) via a flexible linker (e.g. polypeptide linker) that leads to less structural rigidity or stiffness (e.g. between or amongst the ABD and Fc domain) compared to a conventional (e.g. wild-type full length human IgG) antibody. For example, the multispecific protein may have a structure or a flexible linker between the NKp46 ABD and constant domain or Fc domain that permits an increased range of domain motion compared to the two ABDs in a conventional (e.g. wild-type full length human IgG) antibody. In particular, the structure or a flexible linker can be configured to confer on the antigen binding sites greater intrachain domain movement compared to antigen binding sites in a conventional human IgG1 antibody. Rigidity or domain motion/interchain domain movement can be determined, e.g., by computer modeling, electron microscopy, spectroscopy such as Nuclear Magnetic Resonance (NMR), X-ray crystallography, or Sedimentation Velocity Analytical ultracentrifugation (AUC) to measure or compare the radius of gyration of proteins comprising the linker or hinge. A test protein or linker may have lower rigidity relative to a comparator protein if the test protein has a value obtained from one of the tests described in the previous sentence differs from the value of the comparator, e.g., an IgG1 antibody or a hinge, by at least 5%, 10%, 25%, 50%, 75%, or 100%. A cytokine can for example be fused to the C-terminus of a CH3 domain by a linker of any of SEQ ID NOS: 20-23.

In one embodiment, the multispecific protein may have a structure or a flexible linker between the NKp46 ABD and Fc domain that permits the NKp46 ABD and the ABD which binds to CD20 to have a spacing between said ABDs comprising less than about 80 angstroms, less than about 60 angstroms or ranges from about 40-60 angstroms.

At its C-terminus, an Fc domain (or a CH3 domain thereof) can be connected to the N-terminus of a NKp46 ABD or a cytokine polypeptide via a polypeptide linker, for example a glycine-serine-containing linker, optionally a linker having the amino acid sequence STGS (SEQ ID NO: 15).

In certain embodiments, a CH1 or CL domain of a Fab (e.g. of an NKp46 ABD) is fused at its C-terminus to the N-terminus of the cytokine via a flexible polypeptide linker, for example a glycine-serine-containing linker. Preferably, the linker will have a chain length of at least 4 amino acid residues, optionally the linker has a length of 5, 6, 7, 8, 9 or 10 amino acid residues.

In certain embodiments, the NKp46 ABD is placed C-terminal to the Fc domain, and the NKp46 is positioned between an Fc domain and the cytokine polypeptide in the multispecific protein. The NKp46 ABD will be connected or fused at its N-terminus (at the N-terminus of a VH or a VL domain) to the C-terminus of the Fc domain via a linker (e.g. a glycine and serine containing linker, a linker having the sequence STGS, a flexible polypeptide linker) of sufficient length to enable the NKp46 binding ABD to fold and/or adopt an orientation in such a way as to permit binding to Nkp46 at the surface of an NK cell, while at the same time possesses a sufficient distance and range of motion relative to the adjacent Fc domain (or more generally to rest of the multispecific protein) such that the Fc domain can also simultaneously be found by CD16 expressed at the surface of the same NK cell. Additionally, when the NKp46 ABD is placed between an Fc domain and an cytokine polypeptide in the multispecific protein, the C-terminus of a VH or VL of an scFv NKp46 ABD, or the CH1 or CL domain of a Fab NKp46 ABD will be connected or fused to the N-terminus of the cytokine polypeptide via a flexible linker (e.g. a flexible polypeptide linker) of sufficient length to enable the NKp46 binding ABD to fold and/or adopt an orientation in such a way as to permit binding to Nkp46 at the surface of an NK cell, while at the same time providing a sufficient distance and range of motion relative to the adjacent cytokine polypeptide such that the cytokine polypeptide can also simultaneously be bound by its cytokine receptor expressed at the surface of the NK cell. Preferably, the linker will have a chain length of at least 4 amino acid residues, optionally the linker has a length of 5, 6, 7, 8, 9 or 10 amino acid residues.

In tandem variable regions (e.g. scFv), two V domains (e.g. a V_{H} domain and V_{L} domains are generally linked together by a linker of sufficient length to enable the ABD to fold in such a way as to permit binding to the antigen for which the ABD is intended to bind. Examples of linkers include linkers comprising glycine and serine residues, e.g., the amino acid sequence GEGTSTGSGGSGGSGGAD (SEQ ID NO: 96). In another specific embodiment, the V_{H} domain and V_{L} domains of an scFv are linked together by the amino acid sequence (G₄S)₃.

In one embodiment, a (poly)peptide linker used to link a VH or VL domain of an scFv to a CH2 domain of an Fc domain comprises a fragment of a CH1 domain or CL domain and/or hinge region. For example, an N-terminal amino acid sequence of CH1 can be fused to a variable domain in order to mimic as closely as possible the natural structure of a wild-type antibody. In one embodiment, the linker comprises an amino acid sequence from a hinge domain or an N-terminal CH1 amino acid. In one embodiment, the linker peptide mimics the regular VK-CK elbow junction, e.g., the linker comprises or consists of the amino acid sequence RTVA.

In one embodiment, the hinge region used to connect the C-terminal end of a CH1 or CK domain (e.g. of a Fab) with the N-terminal end of a CH2 domain will be a fragment of a hinge region (e.g. a truncated hinge region without cysteine residues) or may comprise one or more amino acid modifications which remove (e.g. substitute by another amino acid, or delete) a cysteine residue, optionally both cysteine residues in a hinge region. Removing cysteines can be useful to prevent undesired disulfide bond formation, e.g., the formation of disulfide bridges in a monomeric polypeptide.

A “hinge” or “hinge region” or “antibody hinge region” herein refers to the flexible polypeptide or linker between the first and second constant domains of an antibody. Structurally, the IgG CH1 domain ends at EU position 220, and the IgG CH2 domain begins at residue EU position 237. Thus for an IgG the hinge generally includes positions 221 (D221 in IgG1) to 236 (G236 in IgG1), wherein the numbering is according to the EU index as in Kabat. References to specific amino acid residues within constant region domains found within the polypeptides shall be, unless otherwise indicated or as otherwise dictated by context, be defined according to Kabat, in the context of an IgG antibody.

For example a hinge domain may comprise the amino acid sequences: DKTHTCPPCP (SEQ ID NO: 5), or an amino acid sequence at least 60%, 70%, 80% or 90% identical thereto; EPKSCDKTHTCPPCP (SEQ ID NO: 13), or an amino acid sequence at least 60%, 70%, 80% or 90% identical thereto; or EPKSCDKTHS (SEQ ID NO: 19), or an amino acid sequence at least 60%, 70%, 80% or 90% identical thereto.

Polypeptide chains that dimerize and associate with one another via non-covalent bonds may or may not additionally be bound by an interchain disulfide bond formed between respective CH1 and Cκ domains, and/or between respective hinge domains on the chains. CH1, Cκ and/or hinge domains (or other suitable linking amino acid sequences) can optionally be configured such that interchain disulfide bonds are formed between chains such that the desired pairing of chains is favored and undesired or incorrect disulfide bond formation is avoided. For example, when two polypeptide chains to be paired each possess a CH1 or Cκ adjacent to a hinge domain, the polypeptide chains can be configured such that the number of available cysteines for interchain disulfide bond formation between respective CH1/Cκ-hinge segments is reduced (or is entirely eliminated). For example, the amino acid sequences of respective CH1, Cκ and/or hinge domains can be modified to remove cysteine residues in both the CH1/Cκ and the hinge domain of a polypeptide; thereby the CH1 and Cκ domains of the two chains that dimerize will associate via non-covalent interaction(s).

In another example, the CH1 or Cκ domain adjacent to (e.g., N-terminal to) a hinge domain comprises a cysteine capable of interchain disulfide bond formation, and the hinge domain which is placed at the C-terminus of the CH1 or Cκ comprises a deletion or substitution of one or both cysteines of the hinge (e.g. Cys 239 and Cys 242, as numbered for human IgG1 hinge according to Kabat).

In another example, the CH1 or Cκ domain adjacent (e.g., N-terminal to) a hinge domain comprises a deletion or substitution at a cysteine residue capable of interchain disulfide bond formation, and the hinge domain placed at the C-terminus of the CH1 or Cκ comprises one or both cysteines of the hinge (e.g. Cys 239 and Cys 242, as numbered for human IgG1 hinge according to Kabat).

In another example, a hinge region is derived from an IgM antibody. In such embodiments, the CH1/CK pairing mimics the Cμ2 domain homodimerization in IgM antibodies. For example, the CH1 or Cκ domain adjacent (e.g., N-terminal to) a hinge domain comprises a deletion or substitution at a cysteine capable of interchain disulfide bond formation, and an IgM hinge domain which is placed at the C-terminus of the CH1 or Cκ comprises one or both cysteines of the hinge.

### Activity Testing

A multispecific protein can be assessed for biological activity, e.g., antigen binding, ability to elicit proliferation of NK cells, ability to elicit target cell lysis by NK and/or elicit activation of NK cells, including any specific signaling activities elicited thereby, for example cytokine production or cell surface expression of markers of activation. In one embodiment, provided are methods of assessing the biological activity, e.g., antigen binding, ability to elicit target cell lysis and/or specific signaling activities elicited thereby, of a multispecific protein of the disclosure. It will be appreciated that when the specific contribution or activity of one of the components of the multispecific protein is to be assessed (e.g. an NKp46 binding ABD, antigen-of-interest binding ABD, an Fc domain, cytokine receptor ABD, etc.), the multispecific format can be produced in a suitable format which allows for assessment of the component (e.g. domain) of interest. The present disclosure also provides such methods, for use in testing, assessing, making and/or producing a multispecific protein. For example, where the contribution or activity of an cytokine is assessed, the multispecific protein can be produced as a protein having the cytokine and another protein in which the cytokine is modified to delete it or otherwise modulate its activity (e.g., wherein the two multispecific proteins otherwise have the same or comparable structure), and tested in an assay of interest. For example, where the contribution or activity of an anti-NKp46 ABD is assessed, the multispecific protein can be produced as a protein having the ABD and another protein in which the ABD is absent or is replaced by an ABD that does not bind NKp46 (e.g., an ABD that binds an antigen not present in the assay system), wherein the two multispecific proteins otherwise have the same or comparable structure, and the two multispecific proteins are tested in an assay of interest. In another example, where the contribution or activity of an anti-CD20 ABD is assessed, the multispecific protein can be produced as a protein having the ABD and another protein in which the ABD is absent or is replaced by an ABD that does not bind CD20 (e.g., an ABD that binds an antigen not present in the assay system, an ABD that bind to a different tumor antigen), wherein the two multispecific proteins otherwise have the same or comparable structure, and the two multispecific proteins are tested in an assay of interest.

In one aspect of any embodiment described herein, the multispecific protein is capable of inducing activation of an NKp46-expressing cell (e.g. an NK cell, a reporter cell) when the protein is incubated in the presence of the NKp46-expressing cell (e.g. purified NK cells) and a target cell (e.g. tumor cell) that expresses CD20.

In one aspect of any embodiment described herein, the multispecific protein is capable of inducing NKp46 signaling in an NKp46-expressing cell (e.g. an NK cell, a reporter cell) when the protein is incubated in the presence of an NKp46-expressing cell (e.g. purified NK cells) and a target cell that expresses the antigen of interest). In one aspect of any embodiment described herein, the multispecific protein is capable of inducing CD16A signaling in an CD16A and NKp46-expressing cell (e.g. an NK cell, a reporter cell) when the protein is incubated in the presence of a CD16A and NKp46-expressing cell (e.g. purified NK cells) and a target cell that expresses CD20).

Optionally, NK cell activation or signaling in characterized by the increased expression of a cell surface marker of activation, e.g. CD107, CD69, Sca-1 or Ly-6A/E, KLRG1, etc.

In one aspect of any embodiment described herein, the multispecific protein is capable of inducing an increase of CD137 present on the cell surface of an NKp46- and/or a CD16-expressing cell (e.g. an NK cell, a reporter cell) when the protein is incubated in the presence of the NKp46- and/or a CD16-expressing cell (e.g. purified NK cells), optionally in the absence of target cells.

In one aspect of any embodiment described herein, the multispecific protein is capable of activating or enhancing the proliferation of NK cells by at least 10-fold, at least 50-fold, or at least 100-fold compared to the same multispecific protein lacking the cytokine receptor ABD (e.g. the CD122 ABD). Optionally the multispecific protein displays an EC50 for activation or enhancing the proliferation of NK cells that is at least 10-fold, 50-fold or 100-fold lower than its EC50 for activation or enhancing the proliferation of CD25-expressing T cells.

In one aspect of any embodiment described herein, the multispecific protein is capable of activating or enhancing the proliferation of NK cells over CD25-expressing T cells, by at least 10-fold, at least 50-fold, or at least 100-fold. Optionally, the CD25 expressing T cells are CD4 T cells, optionally Treg cells, or CD8 T cells.

Activation or enhancement of proliferation via cytokine receptor in cells (e.g. NK cells, CD4 T cells, CD8 T cells or Treg cells) by the cytokine receptor ABD-containing protein can be determined by measuring the expression of pSTAT or the cell proliferation markers (e.g. Ki67) in said cells following the treatment with the multispecific protein. Activation or enhancement of proliferation via the IL-2R pathway in cells (e.g. NK cells, CD4 T cells, CD8 Tcells or Treg cells) by the CD122 ABD-containing protein can be determined by measuring the expression of pSTAT5 or the cell proliferation marker Ki67 in said cells following the treatment with the multispecific protein. IL-2 and IL-15 lead to the phosphorylation of the STAT5 protein, which is involved in cell proliferation, survival, differentiation and apoptosis. Phosphorylated STAT5 (pSTAT5) translocates into the nucleus to regulate transcription of the target genes including the CD25. STAT5 is also required for NK cell survival and NK cells are tightly regulated by the JAK-STAT signaling pathway. In one aspect of any embodiment described herein, the multispecific protein is capable of inducing STAT5 signaling in an NKp46-expressing cell (e.g. an NK cell) when the protein is incubated in the presence of an NKp46-expressing cell (e.g. purified NK cells). In one aspect of any embodiment described herein, the multispecific protein is capable of causing an increase of expression of pSTAT5 in NK cells over CD25-expressing T cells, by at least 10-fold, at least 50-fold, or at least 100-fold. Optionally the multispecific protein displays an EC₅₀ for induction of expression of pSTAT5 in NK cells that is at least 10-fold, 50-fold or 100-fold lower than its EC50 for induction of expression of pSTAT5 in CD25-expressing T cells.

Activity can be measured for example by bringing NKp46-expressing cells (or CD25-expressing cells, depending on the assay) into contact with the multispecific polypeptide, optionally further in presence of target cells (e.g. tumor cells). In some embodiments, activity is measured for example by bringing target cells and NK cells (i.e. NKp46-expressing cells) into contact with one another, in presence of the multispecific polypeptide. The NKp46-expressing cells may be employed either as purified NK cells or NKp46-expressing cells, or as NKp46-expressing cells within a population of peripheral blood mononuclear cell (PBMC). The target cells can be cells expressing the antigen of interest, optionally tumor cells.

In one example, the multispecific protein can be assessed for the ability to cause a measurable increase in any property or activity known in the art as associated with NK cell activity, respectively, such as marker of cytotoxicity (CD107) or cytokine production (for example IFN-γ or TNF-α), increases in intracellular free calcium levels, the ability to lyse target cells, for example in a redirected killing assay, etc.

In the presence of target cells (target cells expressing the antigen of interest) and NK cells that express NKp46, the multispecific protein will be capable of causing an increase in a property or activity associated with NK cell activity (e.g. activation of NK cell cytotoxicity, CD107 expression, IFNγ production, killing of target cells) in vitro. For example, a multispecific protein according to the invention can be selected based on its ability to increase an NK cell activity by more than about 20%, preferably by least about 30%, at least about 40%, at least about 50%, or more compared to that achieved with the same effector: target cell ratio with the same NK cells and target cells that are not brought into contact with the multispecific protein, as measured by an assay that detects NK cell activity, e.g., an assay which detects the expression of an NK activation marker or which detects NK cell cytotoxicity, e.g., an assay that detects CD107 or CD69 expression, IFNγ production, or a classical in vitro chromium release test of cytotoxicity. Examples of protocols for detecting NK cell activation and cytotoxicity assays are described in the Examples herein, as well as for example, in Pessino et al, J. Exp. Med, 1998, 188 (5): 953-960; Sivori et al, Eur J Immunol, 1999. 29:1656-1666; Brando et al, (2005) J. Leukoc. Biol. 78:359-371; EI-Sherbiny et al, (2007) Cancer Research 67(18):8444-9; and Nolte-'t Hoen et al, (2007) Blood 109:670-673). In a classical in vitro chromium release test of cytotoxicity, the target cells are labeled with ⁵¹Cr prior to addition of NK cells, and then the killing is estimated as proportional to the release of ⁵¹Cr from the cells to the medium, as a result of killing. Optionally, a multispecific protein according to the invention can be selected for or characterized by its ability to have greater ability to induce NK cell activity towards target cells, i.e., lysis of target cells compared to a conventional human IgG1 antibody that binds to the same antigen of interest, as measured by an assay of NK cell activity (e.g. an assay that detects NK cell-mediated lysis of target cells that express the antigen of interest).

As shown herein, a multispecific protein, the different ABDs contribute to the overall activity of the multispecific protein that ultimately manifests itself in potent anti-tumor activity in vivo. Testing methods exemplified herein allow the in vitro assessment of the activities of the different individual ABDs of the multispecific protein by making variants of the multispecific protein that lack a particular ABD and/or using cells that lack receptors for the particular ABD. As shown herein, a multispecific protein according to the disclosure, when it does not comprise the cytokine receptor ABD (e.g. the CD122 ABD) and when it possesses an Fc domain that does not bind CD16, does not, substantially induce NKp46 signaling (and/or NK activation that results therefrom) of NK cells when the protein is not bound to the antigen of interest on target cells (e.g. in the absence of the antigen of interest and/or target cells). Thus, the monovalent NKp46 binding component of the multispecific protein does not itself cause NKp46 signaling. Accordingly, in the case of multispecific proteins possessing an Fc domain that binds CD16, such multispecific protein can be produced in a configuration where the cytokine receptor ABD (e.g. CD122 ABD) is inactivated (e.g. modified, masked or deleted, thereby eliminating its ability to binds IL-2Rs) and the protein can be assessed for its ability to elicit NKp46 signaling or NKp46-mediated NK cell activation by testing the effect of this multispecific protein on NKp46 expression, by CD16-negative NK cells. The multispecific protein can optionally be characterized as not substantially causing (or increasing) NKp46 signaling by an NKp46-expressing, CD16-negative cell (e.g. a NKp46⁺CD16⁻ NK cell, a reporter cell) when the multispecific protein is incubated with such NKp46-expressing, CD16-negative cells (e.g., purified NK cells or purified reporter cells) in the absence of target cells.

In one aspect of any embodiment herein, a multispecific protein can for example be characterized by:
(a) capable of inducing cytokine receptor (e.g. CD122) signaling (e.g., as determined by assessing STAT signaling, for example assessing STAT phosphorylation) in an NKp46-expressing cell (e.g. an NK cell) when the multispecific protein is incubated in the presence of an NKp46-expressing cell (e.g. purified NK cells);
(b) being capable of inducing NK cells that express NKp46 (and optionally further CD16) to lyse target cells, when incubated in the presence of the NK cells and CD20 expressing cells; and
(c) lack of NK cell activation or cytotoxicity and/or lack of agonist activity at NKp46 when incubated with NK cells (optionally CD16-negative NK cells, NKp46-expressing NK cells that do not express CD16), in the absence of target cells, optionally wherein the NK cells are purified NK cells, when the multispecific protein is modified to lack the cytokine receptor ABD (e.g. CD122 ABD) or comprises an inactivated cytokine receptor ABD.

### Uses of Compounds

In one aspect, provided is the use of any of the multispecific proteins and/or cells which express the proteins (or a polypeptide chain thereof) for the manufacture of a pharmaceutical preparation for the treatment, prevention or diagnosis of a disease in a mammal in need thereof. Provided also are the use any of the compounds defined above as a medicament or an active component or active substance in a medicament. In a further aspect the invention provides methods for preparing a pharmaceutical composition containing a compound as defined herein, to provide a solid or a liquid formulation for administration (e.g., by subcutaneous or intravenous injection). Such a method or process at least comprises the step of mixing the compound with a pharmaceutically acceptable carrier.

In any aspect herein, the multispecific protein according to the disclosure can be advantageously administered at a dose of 1 μg/kg to 1 mg/kg body weight, optionally 0.05-0.5 mg/kg body weight. The multispecific protein can be advantageously administered 1-4 times per month, preferably 1-2 times per month, for instance once per week, once every two weeks, once every three weeks or once every four weeks. Optionally, administration is by intravenous infusion of subcutaneous administration.

In one aspect, provided is a method to treat, prevent or more generally affect a predefined condition in an individual or to detect a certain condition by using or administering a multispecific protein or antibody described herein, or a (pharmaceutical) composition comprising same.

For example, in one aspect, the invention provides a method of restoring or potentiating the activity and/or proliferation of NKp46-expressing cells, particularly NKp46⁺ NK cells (e.g. NKp46⁺CD16⁺ NK cells, NKp46⁺CD16⁻ NK cells) in a patient in need thereof (e.g. a patient having a cancer), comprising the step of administering a multispecific protein described herein to said patient. In one aspect, the invention provides a method of selectively restoring or potentiating the activity and/or proliferation of NK cells of over CD25-expressing lymphocytes, e.g. CD4 T cells, CD8 T cells, Treg cells. In one embodiment, the method is directed at increasing the activity of NKp46⁺ lymphocytes (e.g. NKp46⁺CD16⁺ NK cells, NKp46⁺CD16⁻ NK cells) in patients having a disease in which increased lymphocyte (e.g. NK cell) activity is beneficial or which is caused or characterized by insufficient NK cell activity, such as a cancer.

In another aspect, the invention provides a method of restoring or potentiating the activity and/or proliferation of NKp46⁺ NK cells (e.g. NKp46⁺CD16⁺ NK cells, NKp46⁺CD16⁻ NK cells) in a patient in need thereof (e.g. a patient having a cancer), comprising the step of contacting cells derived from the patient, e.g., immune cells and optionally target cells expressing an antigen of interest with a multispecific protein according to the invention and reinfusing the multispecific protein treated cells into the patient. In one embodiment, this method is directed at increasing the activity of NKp46⁺ lymphocytes (e.g. NKp46⁺CD16⁺ NK cells) in patients having a disease in which increased lymphocyte (e.g. NK cell) activity is beneficial or which is caused or characterized by insufficient NK cell activity, such as a cancer.

In another embodiment the subject multispecific proteins may be used or administered in combination with immune cells, particularly NK cells, derived from a patient who is to be treated or from a different donor, and these NK cells administered to a patient in need thereof such as a patient having a disease in which increased lymphocyte (e.g. NK cell) activity is beneficial or which is caused or characterized by insufficient NK cell activity, such as a cancer, or a viral or microbial, e.g., bacterial or parasite infection. As NK cells (unlike CAR-T cells) do not express TCRs, these NK cells, even those derived from different donors will not induce a GVHD reaction (see e.g., Glienke et al., “Advantages and applications of CAR-expressing natural killer cells”, Front. Pharmacol. 6, Art. 21:1-6 (2015); Hermanson and Kaufman, Front. Immunol. 6, Art. 195:1-6 (2015)).

In one embodiment, the multispecific protein disclosed herein that mediates NK cell activation, proliferation, tumor infiltration and/or target cell lysis via multiple activating receptors of effector cells, including NKp46, CD16 and CD122, can be used advantageously for treatment of individuals whose effector cells or tumor-infiltrating effector cells (e.g. NKp46+NK cells) cells are hypoactive, exhausted or suppressed, for example a patient who has a significant population of effector cells characterized by the expression and/or upregulation of one or multiple inhibitory receptors (e.g. TIM-3, PD1, CD96, TIGIT, etc.), or the downregulation or low level of expression of CD16 (e.g., presence of elevated proportion of NKp46⁺CD16⁻ NK cells).

The multispecific polypeptides described herein can be used to prevent or treat disorders that can be treated with antibodies, such as cancers, hematological malignancies, and inflammatory or autoimmune disorders.

In one embodiment, a multispecific protein is used to prevent or treat a cancer characterized by CD20 expressing cells selected from the group consisting of: lymphomas (preferably B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma) c) marginal zone lymphomas (including extranodal marginal zone B cell lymphoma (Mucosa-associated lymphatic tissue lymphomas, MALT), nodal marginal zone B cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma) f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B-cell prolymphocytic leukemia, i) plasma cell neoplasms, plasma cell myeloma, multiple myeloma, plasmacytoma j) Hodgkin's disease.

In one embodiment, a multispecific protein is used to prevent or treat a cancer characterized by CD20 expressing cells, wherein said cancer is a solid tumor, preferably a solid, non-hematologic (non-lymphoid) tumor. Such solid tumors include non-hematologic malignancy having B cell involvement, i.e., where B cells are involved in a “protumor” response. Such solid tumors are characterized by palpable tumors, typically at least 0.5 mm in diameter, more typically at least 1.0 mm in diameter. Examples thereof include colorectal cancer, liver cancer, breast cancer, lung cancer, head and neck cancer, stomach cancer, testicular cancer, prostate cancer, ovarian cancer, uterine cancer and others. These cancers may be in the early stages (precancer), intermediate (Stages I and II) or advanced, including solid tumors that have metastasized. These solid tumors will preferably be cancers wherein B cells elicit a protumor response, i.e. the presence of B cells is involved in tumor development, maintenance or metastasis.

In one example, the tumor antigen is an antigen expressed on the surface of a lymphoma cell or a leukemia cell, and the multispecific protein is administered to, and/or used for the treatment of, an individual having a lymphoma or a leukemia.

In one embodiment, the inventive multispecific polypeptides described herein can be used to prevent or treat a cancer characterized by tumor cells that express CD20 to which the multispecific protein of the disclosure specifically binds.

In one aspect, the methods of treatment comprise administering to an individual a multispecific protein described herein in a therapeutically effective amount, e.g., for the treatment of a disease as disclosed herein, for example any of the cancers identified above. A therapeutically effective amount may be any amount that has a therapeutic effect in a patient having a disease or disorder (or promotes, enhances, and/or induces such an effect in at least a substantial proportion of patients with the disease or disorder and substantially similar characteristics as the patient).

The multispecific protein may be used with our without a prior step of detecting the expression of the antigen of interest (e.g. tumor antigen) on target cells in a biological sample obtained from an individual (e.g. a biological sample comprising cancer cells, cancer tissue or cancer-adjacent tissue). In another embodiment, the disclosure provides a method for the treatment or prevention of a cancer in an individual in need thereof, the method comprising:
a) detecting cells (e.g. tumor cells) in a sample from the individual that express an antigen of interest (e.g. the antigen of interest to which the multispecific protein specifically binds via its antigen of interest ABD), and
b) upon a determination that cells which express an antigen of interest are comprised in the sample, optionally at a level corresponding at least to a reference level (e.g. corresponding to an individual deriving substantial benefit from a multispecific protein), or optionally at a level that is increased compared to a reference level (e.g. corresponding to a healthy individual or an individual not deriving substantial benefit from a protein described herein), administering to the individual a multispecific protein of the disclosure that binds to an antigen of interest, to NKp46, to cytokine receptor (e.g. CD122), and optionally to CD16A (e.g., via its Fc domain).

In some embodiments, the multispecific proteins are used to treat a tumor characterized by low levels of cell surface expression of CD20. Accordingly, in, the tumor or cancer can be characterized by cells expressing a low level of CD20. Optionally, the level of CD20 is less than 100,000 copies of CD20 per cancer cell. In some aspects, the level of the tumor antigen is less than 90,000, less than 75,000, less than 50,000, or less than 40,000 copies of CD20 per cancer cell. The uses optionally further comprise detecting the level of CD20 of one or more cancer cells of the subject.

In one embodiment, the disclosure provides a method for the treatment or prevention of a disease (e.g. a cancer) in an individual in need thereof, the method comprising:
a) detecting expression (e.g. cell surface expression) of CD20 polypeptides in cancer cells in a sample from the individual (e.g. in circulation or in the tumor environment), and
b) upon a determination of expression of CD20 in cancer cells, administering to the individual a multispecific protein of the disclosure.

In one embodiment, the disclosure provides a method for the treatment or prevention of a disease (e.g. a cancer) in an individual in need thereof, the method comprising:
a) detecting expression of CD20 polypeptides in cancer cells in a sample from the individual (e.g. in circulation or in the tumor environment), and
b) upon a determination of low expression of CD20 on cancer cells, optionally at a level that is decreased compared to a reference level (e.g., a level that corresponds to a reference level for low cell surface expression; a level that corresponds to an individual not deriving substantial benefit from a therapeutic agent (e.g. an available anti-CD20 antibody such as rituximab, or another approved anti-CD20 agent)), administering to the individual a multispecific protein of the disclosure. Optionally, low expression corresponds to less than 100,000 copies of CD20 per cancer cell. In some aspects, low expression corresponds to less than 90,000, less than 75,000, less than 50,000, or less than 40,000 copies of CD20 per cancer cell.

The multispecific protein may be used with our without a prior step of detecting or characterizing NK cells from an individual to be treated. Optionally, in one embodiment, the invention provides a method for the treatment or prevention of a cancer in an individual in need thereof, the method comprising:
a) detecting NK cells (e.g. tumor-infiltrating NK cells) in a tumor sample from an individual (or within the tumor and/or within adjacent tissue), and
b) upon a determination that the tumor or tumor sample is characterized by a low number or activity of NK cells, optionally at a level or number that is decreased compared to a reference level (e.g. at a level corresponding to an individual deriving no, low or insufficient benefit from a conventional IgG antibody therapy such a conventional IgG1 antibody that binds to the same cancer antigen), administering to the individual a multispecific protein of the disclosure.

In some embodiments, an individual has a tumor characterized by a CD16 (e.g. CD16A) deficient tumor microenvironment. Optionally, the methods of treatment using a multispecific protein comprise a step of detecting the expression level of CD16 in a sample (e.g. a tumor sample) from the individual. Detecting the CD16 optionally comprises detecting the level of CD16A or CD16B. In some aspects, the CD16 deficient microenvironment is assessed in a patient having undergone a hematopoietic stem cell transplantation. Optionally, the CD16 deficient microenvironment comprises a population of infiltrating NK cells, and the infiltrating NK cells have less than 50% expression of CD16 as compared to a control NK cell. In some aspects, the infiltrating NK cells have less than 30%, less than 20%, or less than 10% expression of CD16 as compared to a control NK cell. Optionally, the CD16 deficient microenvironment comprises a population of infiltrating NK cells, and at least 10% of the infiltrating NK cells have reduced expression of CD16 as compared to a control NK cell. In some aspects, at least 20%, at least 30%, or at least 40% of the infiltrating NK cells have reduced expression of CD16 as compared to a control NK cell.

Optionally, in one embodiment, provided is a method for the treatment or prevention of a cancer in an individual in need thereof, the method comprising:
a) detecting CD16 expression in cells (e.g. in tumor-infiltrating NK cells) from a tumor or tumor sample (e.g., tumor and/or within adjacent tissue) from an individual, and
b) upon a determination that the tumor or tumor sample is characterized by a CD16 deficient microenvironment, administering to the individual a multispecific protein of the disclosure.

Optionally, in one embodiment, provided is a method for the treatment or prevention of a cancer in an individual in need thereof, the method comprising:
a) detecting CD16 expression at the surface of NK cells (e.g. tumor-infiltrating NK cells) in a tumor sample from an individual (or within the tumor and/or within adjacent tissue), and
b) upon a determination that the tumor or tumor sample is characterized by an elevated proportion of CD16⁻ NK cells, optionally at a level or number that is increased compared to a reference level, administering to the individual a multispecific protein of the disclosure.

In one embodiment, the disclosure provides a method for the treatment or prevention of a disease (e.g. a cancer) in an individual in need thereof, the method comprising:
a) detecting cell surface expression of one or a plurality inhibitory receptors on immune effector cells (e.g. NK cells, T cells) in a sample from the individual (e.g. in circulation or in the tumor environment), and
b) upon a determination of cell surface expression of one or a plurality inhibitory receptors on immune effector cells, optionally at a level that is increased compared to a reference level (e.g. at a level that is increased compared to a healthy individual, an individual not suffering from immune exhaustion or suppression, or an individual not deriving substantial benefit from a protein described herein), administering to the individual a multispecific protein of the disclosure.

In one embodiment, the disclosure provides a method for the treatment or prevention of a disease (e.g. a cancer) in an individual in need thereof, the method comprising:
a) detecting cell surface expression of NKG2D polypeptides on immune effector cells (e.g. NK cells, T cells) in a sample from the individual (e.g. in circulation or in the tumor environment), and
b) upon a determination of decreased cell surface expression of NKG2D polypeptides on immune effector cells, optionally at a level that is decreased compared to a reference level (e.g. at a level that is increased compared to a healthy individual, an individual not suffering from immune exhaustion or suppression, or an individual not deriving substantial benefit from a protein described herein), administering to the individual a multispecific protein of the disclosure.

In one embodiment, a multispecific protein may be used as a monotherapy (without other therapeutic agents), or in combined treatments with one or more other therapeutic agents.

The multispecific proteins can also be included in kits, for example kits which include:
(i) a pharmaceutical composition containing a multispecific protein,
(ii) a pharmaceutical composition containing a multispecific protein, and a further therapeutic agent, and optionally instructions to administer said multispecific protein with said further therapeutic agent.

A pharmaceutical composition may optionally be specified as comprising a pharmaceutically-acceptable carrier. An multispecific protein may optionally be specified as being present in a therapeutically effective amount adapted for use in any of the methods herein. The kits optionally also can include instructions, e.g., comprising administration schedules, to allow a practitioner (e.g., a physician, nurse, or patient) to administer the composition contained therein to a patient having a cancer. In any embodiment, a kit optionally can include instructions to administer said multispecific protein, optionally other therapeutic agent. The kit also can include a syringe.

Optionally, the kits include multiple packages of the single-dose pharmaceutical compositions each containing an effective amount of a multispecific protein and optionally another therapeutic agent, for a single administration. Instruments or devices necessary for administering the pharmaceutical composition(s) also may be included in the kits. For instance, a kit may provide one or more pre-filled syringes containing an amount of the multispecific protein.

In one embodiment, the present invention provides a kit for treating a cancer or a tumor in a human patient afflicted with cervical cancer, the kit comprising:
(a) a dose of a multispecific protein that binds specifically to human CD20, human NKp46, human CD122, and optionally CD16A, wherein said protein comprises a first (I) polypeptide chain comprising the amino acid sequence of SEQ ID NO: 1, and a second (II) polypeptide chain comprising the amino acid sequence of SEQ ID NO: 70; and/or
(b) optionally, and a dose of another therapeutic agent, and/or
(c) optionally, instructions for using said multispecific protein, and optionally the, said anti-HER antibody and/or said chemotherapy agent in any of the methods described herein.

In some embodiments, the multispecific protein comprises a first (I) polypeptide chain comprising the amino acid sequence of SEQ ID NO: 1, and a second (II) polypeptide chain comprising the amino acid sequence of SEQ ID NO: 70. In some embodiments, the multimeric protein is administered at a dose comprised between 1 μg/kg body weight and 1 mg/kg body weight every one, two, three or four weeks.

The kits may optionally further contain any number of polypeptides and/or other compounds, e.g., 1, 2, 3, 4, or any other number of multispecific proteins and/or other compounds. It will be appreciated that this description of the contents of the kits is not limiting in any way. For example, the kit may contain other types of therapeutic compounds. Optionally, the kits also include instructions for using the polypeptides, e.g., detailing the herein-described methods such as in the detection or treatment of specific disease conditions.

Also provided are pharmaceutical compositions comprising the subject multispecific proteins and optionally other compounds as defined above. A multispecific protein and optionally another compound may be administered in purified form together with a pharmaceutical carrier as a pharmaceutical composition. The form depends on the intended mode of administration and therapeutic or diagnostic application. The pharmaceutical carrier can be any compatible, nontoxic substance suitable to deliver the compounds to the patient. Pharmaceutically acceptable carriers are well known in the art and include, for example, aqueous solutions such as (sterile) water or physiologically buffered saline or other solvents or vehicles such as glycols, glycerol, oils such as olive oil or injectable organic esters, alcohol, fats, waxes, and inert solids. A pharmaceutically acceptable carrier may further contain physiologically acceptable compounds that act for example to stabilize or to increase the absorption of the compounds Such physiologically acceptable compounds include, for example, carbohydrates, such as glucose, sucrose or dextrans, antioxidants, such as ascorbic acid or glutathione, chelating agents, low molecular weight proteins or other stabilizers or excipients One skilled in the art would know that the choice of a pharmaceutically acceptable carrier, including a physiologically acceptable compound, depends, for example, on the route of administration of the composition Pharmaceutically acceptable adjuvants, buffering agents, dispersing agents, and the like, may also be incorporated into the pharmaceutical compositions.

Multispecific proteins according to the invention can be administered parenterally. Preparations of the compounds for parenteral administration must be sterile. Sterilization is readily accomplished by filtration through sterile filtration membranes, optionally prior to or following lyophilization and reconstitution. The parenteral route for administration of compounds is in accord with known methods, e.g. injection or infusion by intravenous, intraperitoneal, intramuscular, intraarterial, or intralesional routes. The compounds may be administered continuously by infusion or by bolus injection. A typical composition for intravenous infusion could be made up to contain 100 to 500 ml of sterile 0.9% NaCl or 5% glucose optionally supplemented with a 20% albumin solution and 1 mg to 10 g of the compound, depending on the particular type of compound and its required dosing regimen. Methods for preparing parenterally administrable compositions are well known in the art.

### EXAMPLES

### Preparation of Multispecific Proteins

The domain structure of an exemplary “T5” format multispecific protein used in the Examples is shown in FIGS. 1 and 2A. FIG. 1 shows domain linkers such as hinge and glycine-serine linkers, and interchain disulfide bridges. The domain structure of the exemplary “T6” format, having a N297S mutation to substantially abolish CD16A binding but otherwise equivalent to format T5, is shown in FIG. 2B. To build the T5 chain L (also referred to as chain 3) the CK domain normally associated with the NKp46-1 VK domain in the NKp46-binding ABD was replaced by a CH1 domain (cross-mab version). The T25 (FIG. 2G) format differs from the T5 format by replacement of the CH1 and CK of the NKp46-binding ABD such that the CK domain normally associated with the NKp46-1 VK domain and the CH1 normally associated with the VH remain associated with therewith. In order to ensure a correct pairing between Chain L (chain 3) and Chain H (chain 1) and formation of a proper disulfide bond between H and L chains, the upper-hinge residues of human IgG1 were added at the C-terminus of CH1 domain of chain L upstream of the linker connecting chain L to IL-2 variant. Other protein formats are shown among FIGS. 2A-2K.

The sequences encoding each polypeptide chain for each multispecific antigen-binding protein were inserted into the pTT-5 vector between the HindIII and BamHI restriction sites. The three vectors (prepared as endotoxin-free midipreps or maxipreps) were used to cotransfect EXPI-293F cells (Life Technologies) in the presence of PEI (37° C., 5% CO₂, 150 rpm). The cells were used to seed culture flasks at a density of 1×10⁶ cells per ml (EXP1293 medium, Gibco). As a reference, for the “T5” constructs, we used a DNA ratio of 0.1 μg/ml (polypeptide chain I), 0.4 μg/ml (polypeptide chain 1l), or 0.8 μg/ml (polypeptide chain III). Valproic Acid (final concentration 0.5 mM), glucose (4 g/L) and tryptone N1 (0.5%) were added. The supernatant was harvested after six days after and passed through a Stericup filter with 0.22 μm pores.

The multispecific antigen-binding proteins were purified from the supernatant following harvesting using rProtein A Sepharose Fast Flow (GE Healthcare, reference 17-1279-03). Size Exclusion Chromatography (SEC) purifications were then performed and the proteins eluted at the expected size were finally filtered on a 0.22 μm device.

### Example 1: CD20-T5-NKCE4 is Potent to Promote IL2R Activation Selectively in NK Cells

The heterotrimeric proteins CD20-1-T5-NKCE4, CD20-2-T5-NKCE4, CD20-3-T5-NKCE4, CD20-4-T5-NKCE4 containing one C-terminal moiety of mutant IL-2 was prepared and assessed for their ability to promote IL-2 R activation on NK cells, CD4 T cells, CD8 T cells and Tregs cells. Said heterotrimeric proteins incorporate a cytokine moiety that is a variant of human interleukin-2 having an amino acid sequence of SEQ ID NO: 26, comprising the deletion of the three first residue and the substitutions R38A, T41A, F42K, conferring a decreased binding affinity for CD25 compared to wild-type human IL-2. Said heterotrimeric proteins incorporate further one Fc domain suitable to binds to CD16A, the VH/VL pair of SEQ ID NOS: 16 and 18 that forms an ABD that binds to a site on the D1/D2 domain of NKp46, and one ABD that binds to CD20. The following D]different ABDs that binds to CD20 were assessed:
CD20-1-T5-NKCE4-v3, that comprise a VH comprising the amino acid sequence of SEQ ID NO: 82 and a VL comprising the amino acid sequence of SEQ ID NO: 83;
CD20-2-T5-NKCE4-v3, that comprise a VH comprising the amino acid sequence of SEQ ID NO: 11 and a VL comprising the amino acid sequence of SEQ ID NO: 3;
CD20-3-T5-NKCE4-v3, that comprise a VH comprising the amino acid sequence of SEQ ID NO: 84 and a VL comprising the amino acid sequence of SEQ ID NO: 85;
CD20-4-T5-NKCE4-v3, that comprise a VH comprising the amino acid sequence of SEQ ID NO: 86 and a VL comprising the amino acid sequence of SEQ ID NO: 87.
Sequences of VH/VL domains used in these examples are presented in table 8 below. Table 8:

| | | |
|---|---|---|
| SEQ ID | CD20-0 | QVQLQQPGAE LVKPGASVKM SCKASGYTFT SYNMHWVKQT |
| NO: 80 | VH | PGRGLEWIGA IYPGNGDTSY NQKFKGKATL TADKSSSTAY |
| | | MQLSSLTSED SAVYYCARST YYGGDWYFNV WGAGTTVTVS A |
| SEQ ID | CD20-0 | QIVLSQSPAI LSASPGEKVT MTCRASSSVS YIHWFQQKPG |
| NO: 81 | VH | SSPKPWIYAT SNLASGVPVR FSGSGSGTSY SLTISRVEAE |
| | | DAATYYCQQW TSNPPTFGGG TKLEIK |
| SEQ ID | CD20-1 | QVQLVQSGAE VKKPGSSVKV SCKASGYAFS YSWINWVRQA |
| NO: 82 | VH | PGQGLEWMGR IFPGDGDTDY NGKFKGRVTI TADKSTSTAY |
| | | MELSSLRSED TAVYYCARNV FDGYWLVYWG QGTLVTVSS |
| SEQ ID | CD20-1 | DIVMTQTPLS LPVTPGEPAS ISCRSSKSLL HSNGITYLYW |
| NO: 83 | VL | YLQKPGQSPQ LLIYQMSNLV SGVPDRFSGS GSGTDFTLKI |
| | | SRVEAEDVGV YYCAQNLELP YTFGGGTKVE IK |
| SEQ ID | CD20-2 | EVOLVESGGG LVQPDRSLRL SCAASGFTFH DYAMHWVRQA |
| NO: 11 | VH | PGKGLEWVST ISWNSGTIGY ADSVKGRFTI SRDNAKNSLY |
| | | LQMNSLRAED TALYYCAKDI QYGNYYYGMD VWGQGTTVTV |
| | | SS |
| SEQ ID | CD20-2 | EIVLTQSPAT LSLSPGREAT LSCRASQSVS SYLAWYQQKP |
| NO: 3 | VL | GQAPRLLIYD ASNRATGIPA RFSGSGSGTF TLTISSLEPE |
| | | DFAVYYCQQR SNWPITFGQG TRLEIK |
| SEQ ID | CD20-3 | EVQLVESGGG LVQPGGSLRL SCAASGYTFT SYNMHWVRQA |
| NO: 84 | VH | PGKGLEWVGA IYPGNGDTSY NQKFKGRFTI SVDKSKNTLY |
| | | LQMNSLRAED TAVYYCARVV YYSNSYWYFD VWGQGTLVTV SS |
| SEQ ID | CD20-3 | DIQMTQSPSS LSASVGDRVT ITCRASSSVS YMHWYQQKPG |
| NO: 85 | VL | KAPKPLIYAP SNLASGVPSR FSGSGSGTDF TLTISSLQPE |
| | | DFATYYCQQW SFNPPTFGQG TKVEIK |
| SEQ ID | CD20-4 | QVQLVQSGAE VKKPGASVKV SCKASGYTFTS YNMHWVRQAP |
| NO: 86 | VH | GQGLEWMGAI YPGNGDTSYN QKFQGRVTIT ADKSISTAYM |
| | | ELSSLRSEDT AVYYCARSTYY GGDWYFNVWG AGTLVTVSS |
| SEQ ID | CD20-4 | QIVLTQSPSS LSASVGDRVT ITCRASSSVS YIHWFQQKPG |
| NO: 87 | VL | KSPKPLIYAT SNLASGVPVR FSGSGSGTDY TLTISSLQPE |
| | | DFATYYCQQW TSNPPTFGGG TKVEIK |

The heterotrimeric proteins were constructed according to the T5 protein format, as shown in FIGS. 1 and 2A.

Briefly, 1M/well of purified PBMC were seeded in 96-well plate and treated with 5 increasing doses of CD20-1-T5-NKCE4, CD20-2-T5-NKCE4, CD20-3-T5-NKCE4, CD20-4-T5-NKCE4 or recombinant IL-2 (dose from 1.33×10⁻⁵ M to 133 nM) for 20 min at 37° C., 5.5% CO₂ in incubator. STAT5 phosphorylation was then analyzed by flow cytometry on NK cells (CD3−CD56+), CD8 T cells (CD3+CD8+), CD4 T cells (CD3+CD4+ FoxP3−) and Tregs (gated on CD3+CD4+CD25+ FoxP3+).

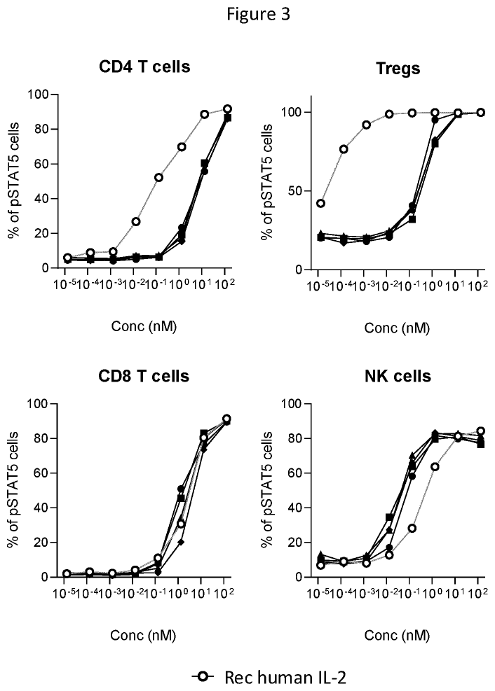

Results are shown in FIG. 3 showing % of pSTAT5 cells among NK cells, CD4 T cells, CD8 T cells, and Tregs cells on the y-axis and concentration of test protein on the x-axis. While recombinant human IL-2 promotes activation of IL-2 receptor on each tested cells, CD20-T5-NKCE4 displayed a significant lower activation of CD4 T cells and Tregs cells. IL-2R activation on CD8 T cell by CD20-T5-NKCE4 was on a comparable level than recombinant IL-2. However, CD20-T5-NKCE4 resulted in an approximately 1-log increase in percent of pSTAT5+ cells among the NK cells, compared to recombinant IL-2 on NK cells. Therefore CD20-T5-NKCE4 proteins permit a selective activation of NK cells over Treg cells, CD4 T cells and CD8 T cells.

### Example 2: CD20-2-T5-NKCE4 Binding to RAJI Tumor Cells

The heterotrimeric proteins CD20-1-T5-NKCE4-v3, CD20-2-T5-NKCE4-v3, CD20-3-T5-NKCE4-v3, CD20-4-T5-NKCE4-v3, as described in example 1, were assessed by flow cytometry for their ability to bind to RAJI cells. The different proteins were tested for binding to RAJI cells (CD20-expressing cells).

Briefly, 10⁵ RAJI cells were incubated 10 minutes at 4° C. with normal mouse serum for saturation. RAJI cells were then incubated with increasing doses of CD20-T5-NKCE4 for 30 min at 4° C. After 2 washes, CD20-T5-NKCE4 bound to RAJI cells were revealed with a secondary Goat anti human IgG (H+L) APC antibody and flow cytometry analysis.

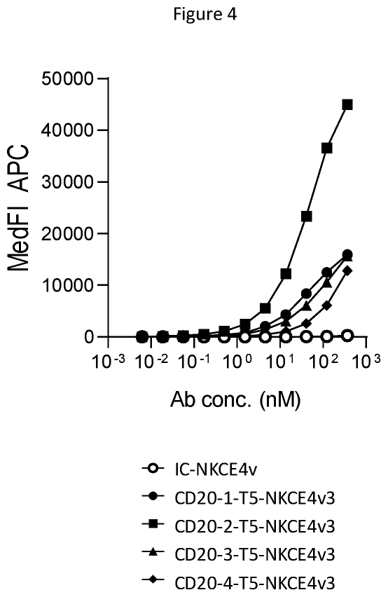

Results are shown in FIG. 4 showing the median fluorescence intensity measured is shown on the y-axis and concentration of test protein is shown on the x-axis. The binding affinity of the different heterotrimeric proteins for the tumor cells was comparable, however with the exception of CD20-2-T5-NKCE4 which exhibited a significantly stronger binding to RAJI tumor cells compared to the other proteins.

The CD20-2 ABD conferred a particularly strong binding to the NKCE4 protein.

### Example 3: CD20-2-T5 NKCE4-v2A Binds Selectively to CD122

The heterotrimeric protein CD20-2-T5A-NKCE4-v2A, comprising a first polypeptide chain of SEQ ID NO: 91, a second polypeptide chain of SEQ ID NO: 9, and a third polypeptide chain of SEQ ID NO: 17 was assessed for its ability to bind IL-2 receptors CD25, CD122 and CD132 through an SPR-Biacore instrument.

Briefly, a biacore instrument was used with a CM5 chip comprising an immobilized anti-His antibody. At the beginning of each cycle, ligand HuCD122-His (cycle 1), HuCD25-His (cycle 2) or HuCD132-His (cycle 4) was injected at a dilution of 15 mg/ml to be captured on the chip. The protein CD20-2-T5A-NKCE4-v2A (1 μM) was then injected during 120 s with a flow of 10 μL/min. The interaction between CD20-2-T5A-NKCE4-v2A and HuCD25-His, HuCD122-His or HuCD132-His was studied with a dissociation time of 600 s. The chip was then regenerated with NaOH 10 mM at a flow of 40 μL/min during 10 sec.

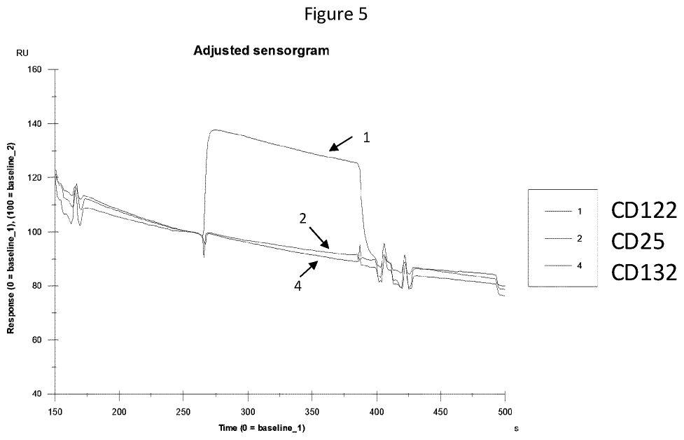

Part of the sensogram of this experiment is shown in FIG. 5. This sensogram exposed the response measured during the injection of the CD20-2-T5A-NKCE4-v2A protein. In details, during cycle 1 (ligand=HuCD122-His), injection of CD122-His at 15 μg/mL during 120 s at 10 μL/min induced a response of +154.0 RU. Then the injection of CD20-2-T5A-NKCE4-v2A at 1 μM during 120 s at 10 μL/min induced a response of +34.4 RU. Finally, a residual response lower than 1 RU was observed after the regeneration with NaOH 10 mM during 10 s at 40 μL/min. During cycle 2 (ligand=HuCD25-His), injection of CD25-His at 15 μg/mL during 120 s at 10 μL/min produced a response of +80 RU. Then the injection of CD20-2-T5A-NKCE4-v2A at 1 μM during 120 s at 10 μL/min induced a response of −7 RU. Endly, a residual response lower than 5 RU was observed after regeneration with NaOH 10 mM during 10 s at 40 μL/min. During cycle 4 (ligand=HuCD132-His), injection of CD132-His at 15 μg/mL during 120 s at 10 μL/min induced a response of +41 RU. Then the injection of CD20-2-T5A-NKCE4-v2A at 1 μM during 120 s at 10 μL/min induced a response of −6 RU. A residual response lower than 5 RU was observed after regeneration with NaOH 10 mM during 10 s at 40 μL/min.

As shown on FIG. 5, no binding between CD20-2-T5A-NKCE4-v2A and receptors CD25 (interleukin 2 receptor alpha) and CD132 (interleukin 2 receptor gamma) was observed. The sensogram shows however that CD20-2-T5A-NKCE4-v2A is able to bind to CD122 (interleukin 2 receptor beta).

### Example 4: CD20-2-NKCE4-v2A Affinity Toward CD122

The heterotrimeric proteins CD20-2-T5A-NKCE4-v2, CD20-2-T6AB3-NKCE4-v2A, and the dimeric protein CD20-2-T13A-NKCE4-v2A was produced and their affinity toward CD122 was studied through SPR-Biacore.

CD20-2-T5A-NKCE4-v2 comprises a first polypeptide chain of SEQ ID NO: 91, a second polypeptide chain of SEQ ID NO: 9, a third polypeptide chain of SEQ ID NO: 17. CD20-2-T6AB3-NKCE4-v2A comprises a first polypeptide chain of SEQ ID NO: 92, a second polypeptide chain of SEQ ID NO: 69, a third polypeptide chain of SEQ ID NO: 17. CD20-2-T13A-NKCE4-v2A comprises a first polypeptide chain of SEQ ID NO: 91 and a second polypeptide chain of SEQ ID NO: 70.

Briefly, a biacore instrument was used with a CM5 chip comprising an immobilized anti-His antibody. Ligand HuCD122-His was injected at a dilution of 15 mg/ml to be captured on the chip. Proteins CD20-2-T5A-NKCE4-v2, CD20-2-T6AB3-NKCE4-v2A, CD20-2-T13A-NKCE4-v2A was then injected during 120 s with a flow of 10 μL/min, at a concentrations range from 31.25 nM to 1 μM. The interaction between these proteins and HuCD122-His was studied with a dissociation time of 600 s. The chip was then regenerated with NaOH 10 mM at a flow of 40 μL/min during 10 sec.

Date has been analysed under the steady-state model, that seemed to be the most accurate regarding sensograms appearance. The different KD thus calculated are presented in following table 9.

TABLE 9

| Sample | KD (nM) | Chi² |
|---|---|---|
| CD20-2-T5A-NKCE4-v2 | 1812 | 0.1230 |
| CD20-2-T6AB3-NKCE4-v2A | 2837 | 0.0292 |
| CD20-2-T13A-NKCE4-v2A | 3374 | 0.0563 |

According to steady-state reaction fit, it can be concluded that the formats of NKCE, additionally differing in their cytokine moieties (IL-2v2 having an amino acid sequence of SEQ ID NO: 25 and IL2v2A having an amino acid sequence of SEQ ID NO: 65) display comparable affinity for CD122 (IL2RP).

### Example 5: CD20-2 NKCE4 is the Best Inducer of Cytotoxicity on RAJI Tumor Cells

In this experiment, NKCE proteins were assessed for their ability to induce killing of RAJI tumor cells (CD20+) by NK cells from human donors at effector: target ratio of 10:1 in a standard 4-hour cytotoxicity assay using calcein release as readout.

CD20-T5-NKCE4-v3 proteins were exhibiting several CD20 ABD, as in example 1:
CD20-1-T5-NKCE4-v3, that comprise a VH comprising the amino acid sequence of SEQ ID NO: 91 and a VL comprising the amino acid sequence of SEQ ID NO: 92;
CD20-2-T5-NKCE4-v3, that comprise a VH comprising the amino acid sequence of SEQ ID NO: 11 and a VL comprising the amino acid sequence of SEQ ID NO: 3;
CD20-3-T5-NKCE4-v3, that comprise a VH comprising the amino acid sequence of SEQ ID NO: 93 and a VL comprising the amino acid sequence of SEQ ID NO: 94;
CD20-4-T5-NKCE4-v3, that comprise a VH comprising the amino acid sequence of SEQ ID NO: 95 and a VL comprising the amino acid sequence of SEQ ID NO: 96.
IC-T5-NKCE4-v3, having the same structure than the other proteins, except that the CD20 ABD was substituted by a VH/VL pair that does not bind to a protein present in this experiment.

The heterotrimeric proteins were constructed according to the T5 protein format, as shown in FIGS. 1 and 2A.

Briefly, freshly purified NK cells from healthy donors were cocultured with Raji tumor cells previously loaded with calcein in a 10 to 1 ratio. Cells were incubated with test proteins described above (doses from 6.6×10⁻⁶ to 66 nM) for 4 h at 37° C., 5.5% CO₂ in incubator. Cytotoxicity is monitored by evaluating calcein release.

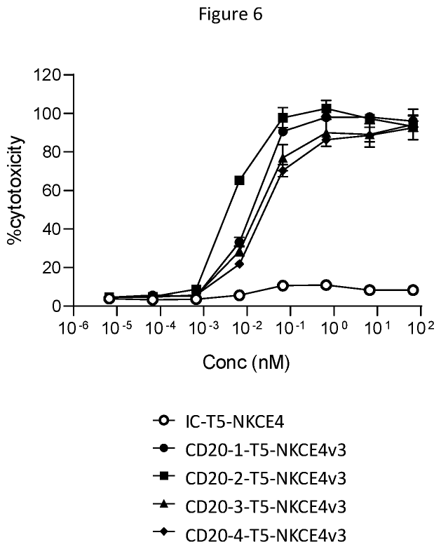

Results are shown in FIG. 6 showing % of cytotoxicity induced by NK cells on the y-axis and concentration of test protein on the x-axis. All CD20-T5-NKCE4-v3 proteins whatever their CD20 ABD, were highly potent in ability to mediate NK cell cytotoxicity toward tumor target cells. However, CD20-2-T5-NKCE4-v3 induced a significantly better induction of NK cell cytotoxicity on RAJI tumor cells.

The different EC50 of cytotoxicity for each molecules were calculated for NK cells isolated from blood of 4 different donors and are presented in following table 10.

TABLE 10

| | CD20-1-T5- | CD20-2-T5- | CD20-3-T5- | CD20-4-T5- |
|---|---|---|---|---|
| | NKCE4-v3 | NKCE4-v3 | NKCE4-v3 | NKCE4-v3 |
| EC50 | 6.6 × 10³ nM +/− | 3.5 × 10³ nM +/− | 14 × 10³ nM +/− | 25 × 10³ nM +/− |
| cytotoxicity | 0.004 | 0.002 | 0.008 | 0.015 |
| (nM) +/− SD | | | | |

### Example 6: CD20-2-T5-NKCE4-v3 Displays Strong Anti-Tumor Activity In Vivo

In this experiment, a single injection of 0.4 μg, 2 μg or 10 μg of NK cell engager proteins CD20-2-T13-NKCE4-v2A or CD20-2-T5-NKCE4 was assessed for its in vivo anti-tumor activity in a murine model of human cancer. CD20-2-T13-NKCE4-v2a is a heterodimeric protein comprising a first polypeptide chain of the amino acid sequence of SEQ ID NO: 1 and a second polypeptide chain of the amino acid sequence of SEQ ID NO: 70, and binds to NKp46, CD122, CD20 and CD16A. CD20-1-T5-NKCE4 is a heterotrimeric protein that comprises a first polypeptide chain of the amino acid sequence of SEQ ID NO: 101, a second polypeptide chain of the amino acid sequence of SEQ ID NO: 102, and a third polypeptide chain of the amino acid sequence of SEQ ID NO: 103 and binds to NKp46, CD122, CD20 and CD16A.

Briefly, CB17 SCID mice were engrafted subcutaneously by 5×10⁶ RAJI cells in matrigel. At day 9 post-engraftment, mice were treated with a single intravenous injection of 0.4, 2 or 10 μg of CD20-2-T13-NKCE4-v2A or CD20-2-T5-NKCE4 and PBS as vehicle. Tumor volume was measured at day 26 post-engraftment.

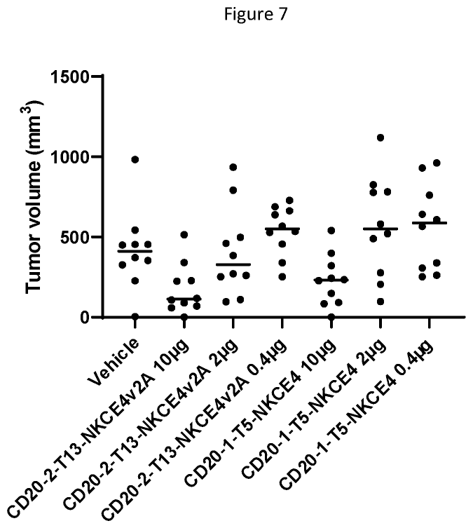

Results are shown in FIG. 7. Each dot represents the tumor volume in an individual animal. A dose of 10 μg of CD20-2-T13-NKCE4-v2A or CD20-2-T5-NKCE4 showed strong efficacy as a single injection compared to vehicle alone.

### Example 7: Different Formats of NKCE4 are Able to Induce Cytotoxicity on RAJI Tumor Cells

In this experiment, several additional formats of NKCE4 all incorporating the CD20-2 binding domain and the NKp46 binding domain of the VH/VL pair of SEQ ID NOS: 16 and 18 were assessed for their ability to induce cytotoxicity on RAJI tumor cells. The test protein included in this experiment were:
CD20-2-T5-NKCE4-v2 is a heterotrimeric protein that comprises a first polypeptide chain of the amino acid sequence of SEQ ID NO: 1, a second polypeptide chain of the amino acid sequence of SEQ ID NO: 9, and a third polypeptide chain of the amino acid sequence of SEQ ID NO: 98. CD20-2-T5-NKCE4-v2 contains from N- to C-terminus, anti-CD20 VH/VL pair (Fab), Fc domain dimer that binds CD16, NKp46 VH/VL pair (Fab), IL2v2.
CD20-2-T5A-NKCE4-v2 is a heterotrimeric protein that comprises a first polypeptide chain of the amino acid sequence of SEQ ID NO: 91, a second polypeptide chain of the amino acid sequence of SEQ ID NO: 9, and a third polypeptide chain of the amino acid sequence of SEQ ID NO: 98. CD20-2-T5A-NKCE4-v2 contains from N- to C-terminus, anti-CD20 VH/VL pair (Fab), Fc domain dimer that binds CD16, NKp46 VH/VL pair (Fab), IL2v2.
CD20-2-T13A-NKCE4-v2 is a heterodimeric protein that comprises a first polypeptide chain of the amino acid sequence of SEQ ID NO: 91, and a second polypeptide chain of the amino acid sequence of SEQ ID NO: 99. CD20-2-T13A-NKCE4-v2 contains from N- to C-terminus, anti-CD20 VH/VL pair (Fab), Fc domain dimer that binds CD16, NKp46 scFv, IL2v2.
CD20-2-T6AB3-NKCE4-v2 is a heterotrimeric protein that comprises a first polypeptide chain of the amino acid sequence of SEQ ID NO: 92, a second polypeptide chain of the amino acid sequence of SEQ ID NO: 69, and a third polypeptide chain of the amino acid sequence of SEQ ID NO: 98. CD20-2-T13AB3-NKCE4-v2 contains from N- to C-terminus, anti-CD20 VH/VL pair (Fab), Fc domain dimer mutated to abolish CD16 binding, NKp46 (Fab), IL2v2.
CD20-2-T14A-NKCE4-v2A is a heterodimeric protein that comprises a first polypeptide chain of the amino acid sequence of SEQ ID NO: 92, and a second polypeptide chain of the amino acid sequence of SEQ ID NO: 71. CD20-2-T14A-NKCE4-v2A contains from N- to C-terminus, anti-CD20 VH/VL pair (Fab), Fc domain dimer mutated to abolish CD16 binding, NKp46 scFv, IL2v2A.
CD20-2-T175-NKCE4-v2 is a heterotrimeric protein that comprises a first polypeptide chain of the amino acid sequence of SEQ ID NO: 77, a second polypeptide chain of the amino acid sequence of SEQ ID NO: 78, and a third polypeptide chain of the amino acid sequence of SEQ ID NO: 100. CD20-2-T175-NKCE4-v2 contains from N- to C-terminus, anti-CD20 VH/VL pair, Fc domain dimer that binds to CD16, NKp46 (Fab), IL2v2A.
CD20-2-T195-NKCE4-v2 is a heterotrimeric protein that comprises a first polypeptide chain of the amino acid sequence of SEQ ID NO: 77, a second polypeptide chain of the amino acid sequence of SEQ ID NO: 79, and a third polypeptide chain of the amino acid sequence of SEQ ID NO: 98. CD20-2-T195-NKCE4-v2 contains from N- to C-terminus, anti-CD20 VH/VL pair, Fc domain dimer that binds to CD16, NKp46 (Fab), IL2v2.

Briefly, freshly purified NK cells from donors were rested overnight in complete medium. Resting NK cells were then cocultured with Raji tumor cells previously loaded with calcein release, in a 10 to 1 ratio. Cells were incubated with test proteins described above (doses from 6.1×10−6 to 61 nM doses from 10−5 to 10² M) for 4 h at 37° C., 5.5% CO2 in incubator. Cytotoxicity is monitored by evaluating calcein release.

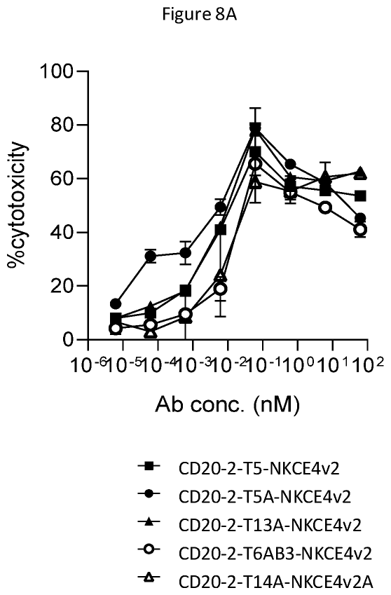

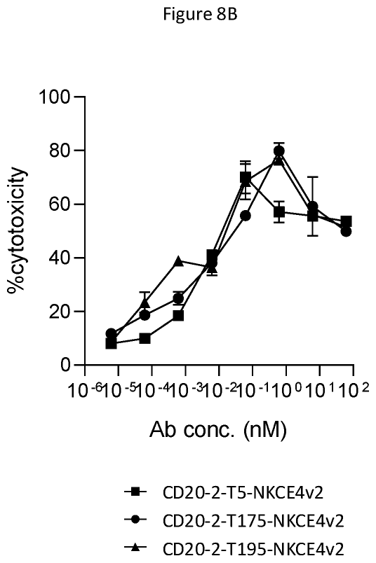

Results are shown in FIGS. 8A and B showing % of cytotoxicity induced by NK cells on the y-axis and concentration of test protein on the x-axis. All NKCE4 proteins whatever their format, were highly potent in ability to mediate NK cell cytotoxicity toward tumor target cells.

### Example 8: Comparison of Induction of IL2R Signalling in NK Cells

In this experiment, the potency of two multispecific protein to induce proliferation of a NK cell line in a bioassay system was evaluated.

The test proteins were:
CD20-2-T13-NKCE4-v2A, a heterodimeric protein that comprises a first polypeptide chain of the amino acid sequence of SEQ ID NO: 1, and a second polypeptide chain of the amino acid sequence of SEQ ID NO: 70. CD20-2-T13-NKCE4-v2A contains from N- to C-terminus, anti-CD20 VH/VL pair (Fab), Fc domain dimer that binds CD16, NKp46 scFv, IL2v2A;
CD20-1-T5-NKCE4-IL2v, a heterotrimeric protein that contains from N- to C-terminus, anti-CD20 VH/VL pair (Fab) according to SEQ ID NOS: 82 and 83, Fc domain dimer that binds CD16, NKp46 scFv, IL2v.

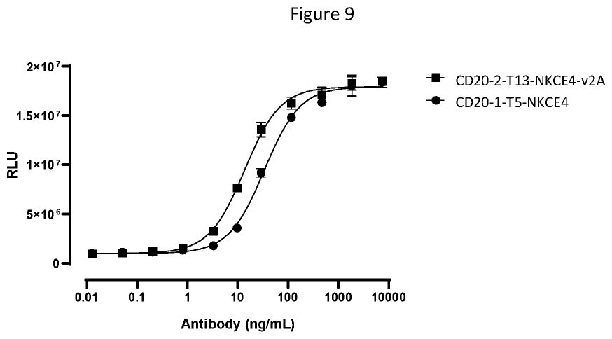

Briefly, 10 000 KHYG-1 cells expressing NKp46 and modified to express high level of CD16 were incubated for 50 h with test molecules in a serial dilution from 7.5 ug/mL to 0.01 ng/mL. Cell proliferation was evaluated with Cell Titer Glo (RLU on the y-axis). Results are shown in FIG. 9. The data showed that CD20-2-T13-NKCE4v2A was more potent than the CD20-1-T5-NKCE4-IL2v multispecific protein to induce NK cell proliferation.

### Example 9: Administration of CD20-NKCE4 to Non-Human Primates

In this experiment, several formats of NKCE4 all incorporating the CD20-1 binding domain and the NKp46 binding domain of the VH/VL pair of SEQ ID NOS: 82 and 83 were tested.

The test proteins included in this experiment were:
CD20-1-T5-NKCE4 is a heterotrimeric protein that comprises a first polypeptide chain of the amino acid sequence of SEQ ID NO: 101, a second polypeptide chain of the amino acid sequence of SEQ ID NO: 102, and a third polypeptide chain of the amino acid sequence of SEQ ID NO: 103. CD20-1-T5-NKCE4 contains from N- to C-terminus, anti-CD20 VH/VL pair (Fab), Fc domain dimer that binds CD16, NKp46 scFv, IL2v;
CD20-1-T6-NKCE4 is a heterotrimeric protein that comprises a first polypeptide chain of the amino acid sequence of SEQ ID NO: 104, a second polypeptide chain of the amino acid sequence of SEQ ID NO: 105, and a third polypeptide chain of the amino acid sequence of SEQ ID NO: 106. CD20-1-T6-NKCE4 contains from N- to C-terminus, anti-CD20 VH/VL pair (Fab), Fc domain dimer mutated to abolish CD16 binding, NKp46 scFv, IL2v.

A first multispecific protein CD20-1-T5-NKCE4 was Fc competent, having a CH2-CH3 domain able to bind to CD16A, whereas a second multispecific protein CD20-1-T6-NKCE4 was Fc non-competent, having a CH2-CH3 domain with mutations that prevented the binding to CD16A. These multispecific proteins were then administered intravenously to non-human primates at a dose of 0.05 mg/kg body weight and 0.5 mg/kg body weight for the Fc competent CD20-1-T5-NKCE4, and at a dose of 0.5 mg/kg for the Fc non-competent CD20-1-T6-NKCE4. Each of these three settings were administered to a cohort comprising four non-human primates (compared to vehicle as control).

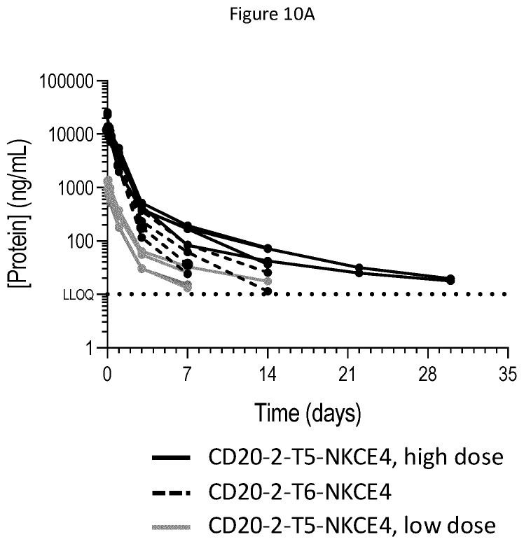

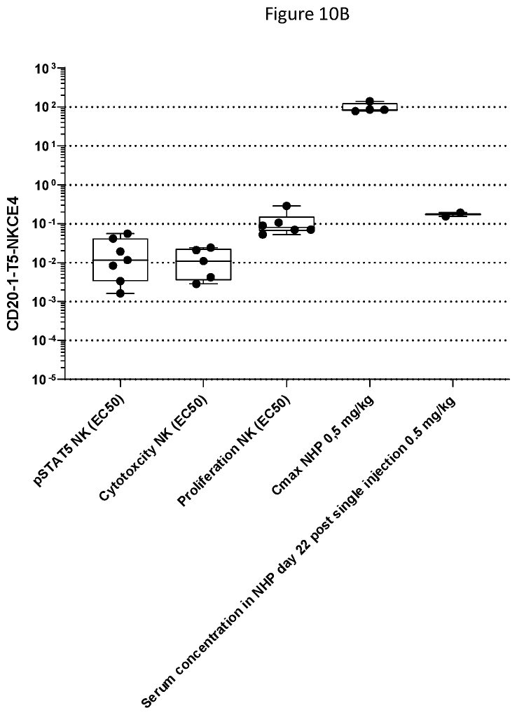

Pharmacokinetic of these multispecific proteins was followed by measuring their sera concentrations over time. Results are shown in FIG. 10A.

The evaluable terminal half-life was calculated for three anti-drug antibody free animals treated with CD20-1-T5-NKCE4 (Fc competent): one at 0.05 mg/kg and two other at 0.5 mg/kg. Terminal half-life for the dose of 0.05 mg/kg was 146 hours (6 days). Terminal half-lives for the dose of 0.5 mg/kg were 311 hours (13 days) and 175 hours (7 days). The results of in-vivo stability show that the multispecific proteins are compatible with an administration to humans that can be for example every 2, 3 or 4 weeks.

The concentrations at which the CD20-1-T5-NKCE4 protein induces expression of pSTAT5 in NK cells, cytotoxicity against NK cells and proliferation of NK cells were determined in vitro in human PBMCs and compared to the maximal serum concentration of CD20-1-T5-NKCE4 and the serum concentration of CD20-1-T5-NKCE4 at day 22 post-injection administered at 0.5 mg/kg body weight. Results are shown in FIG. 10B. The results show that the serum concentration at day 22 of CD20-1-T5-NKCE4 administered at 0.5 mg/kg body weight remained at or above the EC₅₀ values for each of pSTAT5 induction, NK cell-mediated cytotoxicity and NK cell proliferation.

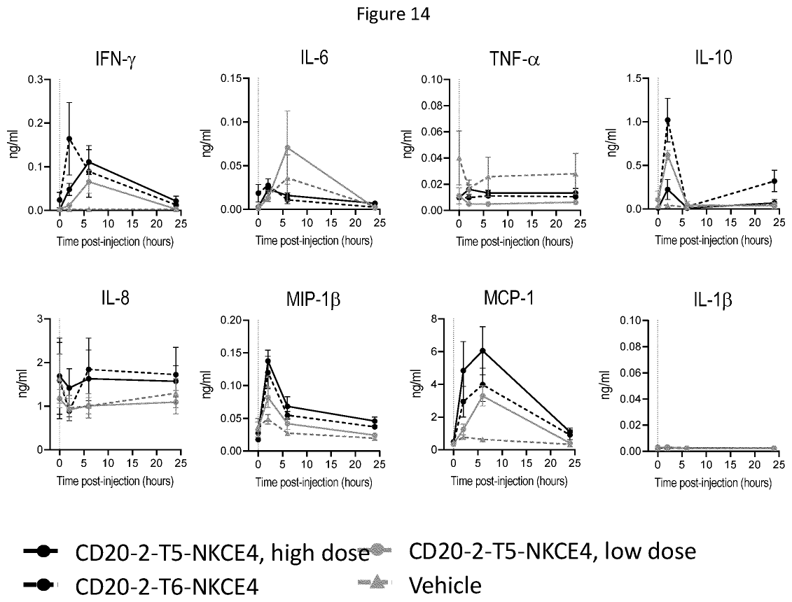

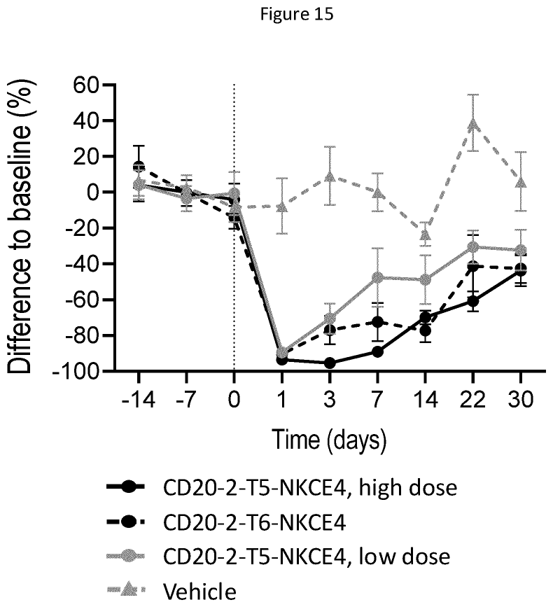

Release of several cytokines (IFN-γ, IL-6, TNF-α, IL-10, IL-8, MIP-1β, MCP-1, IL-1β) was also monitored after injection of multispecific proteins. Results are shown in FIG. 14. A transient increase of release of IFN-γ, IL-6, IL-10, IL-8, MIP-1β, MCP-1 was observed during the first five days after the injection, thereafter decreasing to the baseline. The multispecific protein did not have an impact on TNF-α and IL-1β release. In conclusion, the CD20-specific NKCE4 molecules induced low systemic cytokine release in non-human primates yet provided strong depletion of CD20-expressing B cells, as shown in FIG. 15.

### Example 10: Study of Immune Cell Populations Upon Administration of CD20-NKCE4 to Non Human Primates

Multispecific proteins were administered intravenously or subcutaneously to non-human primates at a dose of 0.5 mg/kg body weight administered every week (i.e. on day 0, 7 and 14 of the experiment) Two non-human primates (2258 and 2261) received their doses of CD20-2-T13-NKCE4-v2A intravenously, whereas one (2262) received its doses by subcutaneous injections.

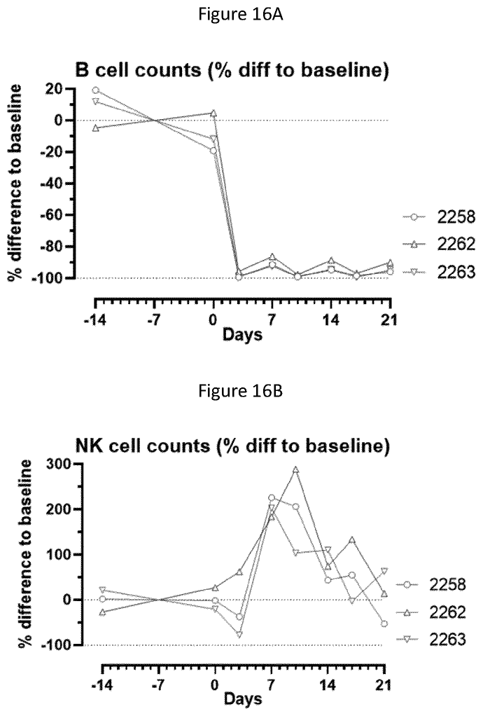

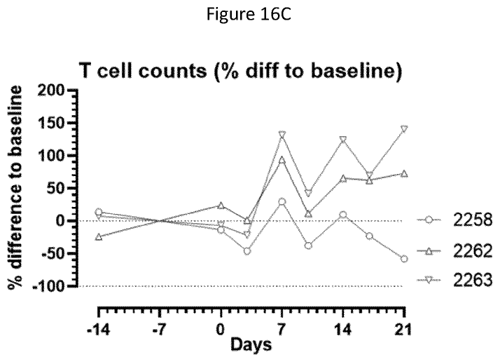

The number of circulating B, T and NK cells was followed over time by flow cytometry. Results are shown on FIGS. 16A, 16B and 16C. The administration of the multispecific protein CD20-2-T13-NKCE4-v2A induced a strong and sustained depletion of B cells (about 100%). In addition, moderate expansion/contraction of T and NK cells population was observed, notably after the first administration (day 0) of multispecific protein, but no depletion was observed for these cell populations.

Depletion of B, T and NK cell subset induced by CD20-2-T13-NKCE4-v2A was further monitored in vitro in human PBMCs and compared to an isotype control multispecific protein that binds to CD16A and NKp46 (IC-T13-NKCE4-v2A), to a multispecific protein that binds to CD20, CD16, NKp46, but does not comprise an IL-2 moiety (CD20-2-F13-NKCE3), and to a control IL-2 protein (IL-2 coupled to His and BirA protein tags).

The tested protein included:
CD20-2-T13-NKCE4-v2A is a heterodimeric protein that comprises a first polypeptide chain of the amino acid sequence of SEQ ID NO: 1, and a second polypeptide chain of the amino acid sequence of SEQ ID NO: 70. CD20-2-T13-NKCE4-v2A contains from N- to C-terminus, anti-CD20 VH/VL pair (Fab), Fc domain dimer that binds CD16, NKp46 scFv, IL2v2A;
IC-T13-NKCE4-v2A, having the same structure than CD20-2-T13-NKCE4-v2A, the CD20 ABD being substituted by an isotype control (CD20 VH/VL replaced by a VH/VL pair that do not bind to any protein present in the experiment).
CD20-2-F13-NKCE3 is a heterodimeric protein that comprises a first polypeptide chain of the amino acid sequence of SEQ ID NO: 1, and a second polypeptide chain of the amino acid sequence of SEQ ID NO: 97. CD20-2-F13-NKCE3 contains from N- to C-terminus, anti-CD20 VH/VL pair (Fab), Fc domain dimer that binds CD16, NKp46 scFv.

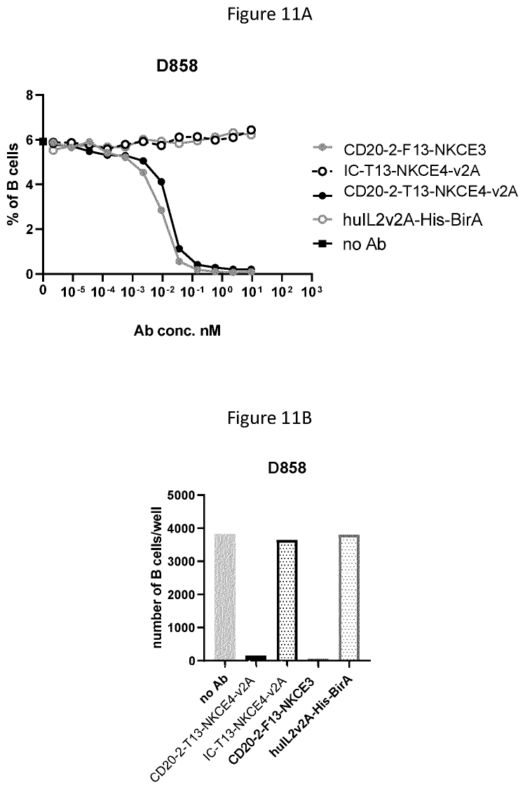

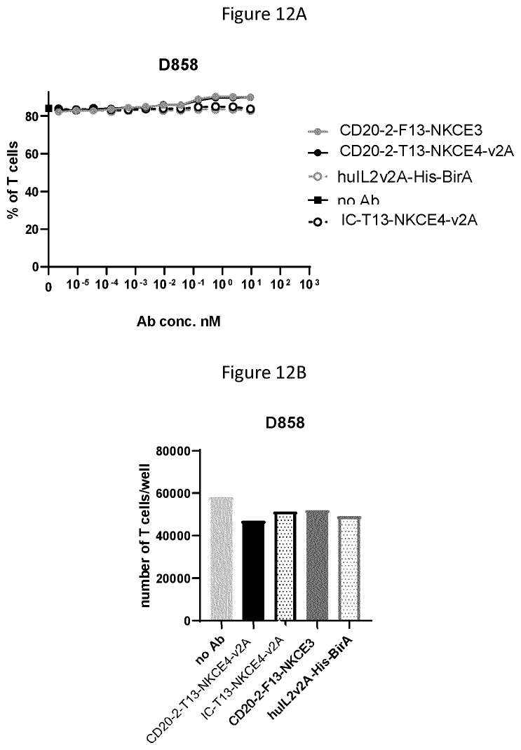

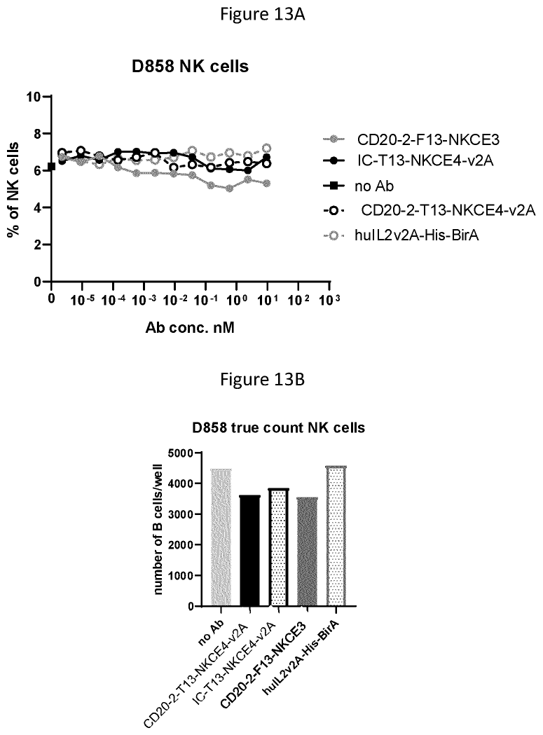

Results are shown in FIGS. 11A and 11B for B cells, in FIGS. 12A and 12B for T cells, and in FIGS. 13A and 13B for NK cells. As shown in FIGS. 11A and 11B, multispecific proteins CD20-2-T13-NKCE4-v2A and CD20-2-F13-NKCE3 at a range of 10 nM were able to deplete almost 100% of B cells. Neither IL2 nor IC-T13-NKCE4-v2A were able to induce B cell depletion. As shown in FIGS. 12A, 12B, 13A and 13B, none of the tested proteins were able to induce significant T cell or NK cell depletion.

The results show that the CD20-2-T13-NKCE4-v2A is highly potent in the ability to deplete B cells, yet without causing fratricidal killing of NK cells.

All headings and sub-headings are used herein for convenience only and should not be construed as limiting the invention in any way. Any combination of the above-described elements in all possible variations thereof is encompassed by the invention unless otherwise indicated herein or otherwise clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate). All methods described herein can be performed in any suitable order unless otherwise indicated herein or otherwise clearly contradicted by context.

The use of any and all examples, or exemplary language (e.g., “such as”) provided herein is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as reference to an element or elements is intended to provide support for a similar aspect or embodiment of the invention that “consists of,” “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

All publications and patent applications cited in this specification are herein incorporated by reference in their entireties as if each individual publication or patent application were specifically and individually indicated to be incorporated by reference.

Although the foregoing invention has been described in some detail by way of illustration and example for purposes of clarity of understanding, it will be readily apparent to one of ordinary skill in the art in light of the teachings of this invention that certain changes and modifications may be made thereto without departing from the spirit or scope of the appended claims.

1-43. (canceled)

44. A multimeric binding protein that binds specifically to human CD20, human NKp46 and human CD122, wherein said protein comprises:
a) a first (I) polypeptide chain comprising the amino acid sequence of SEQ ID NO: 1, and a second (II) polypeptide chain comprising the amino acid sequence of SEQ ID NO: 70;
b) a first (I) polypeptide chain comprising the amino acid sequence of SEQ ID NO: 1, a second (II) polypeptide chain having an amino acid sequence of SEQ ID NO: 9, and a third (III) polypeptide chain comprising the amino acid sequence of SEQ ID 17;
or,
c) a first (I) polypeptide chain comprising the amino acid sequence of SEQ ID NO: 1, a second (II) polypeptide chain having an amino acid sequence of SEQ ID NO: 73, and a third (III) polypeptide chain comprising the amino acid sequence of SEQ ID NO: 74,
or,
d) a first (I) polypeptide chain comprising the amino acid sequence of SEQ ID NO: 66, a second (II) polypeptide chain having an amino acid sequence of SEQ ID NO: 67, and a third (III) polypeptide chain comprising the amino acid sequence of SEQ ID 17.

45. A binding protein comprising a first and a second antigen binding domain (ABD), a cytokine moiety and all or part of an immunoglobulin Fc region or variant thereof, wherein each of said ABDs comprise an immunoglobulin heavy chain variable domain (VH) and an immunoglobulin light chain variable domain (VL), wherein each VH and VL comprises three complementary determining regions (CDR1, CDR2 and CDR3); and wherein:
(i) the first ABD binds specifically to human CD20 and comprises:
a VH1 comprising a CDR1, CDR2 and CDR3 corresponding to the amino acid sequences of SEQ ID NO: 29 (HCDR1), SEQ ID NO: 32 (HCDR2), SEQ ID NO: 35 (HCDR3), and
a VL1 comprising a CDR1, CDR2 and CDR3 corresponding to the amino acid sequences of SEQ ID NO: 38 (LCDR1), SEQ ID NO: 41 (LCDR2), SEQ ID NO: 44 (LCDR3);
(ii) the second ABD binds specifically to human NKp46 and comprises:
a VH2 comprising a CDR1, CDR2 and CDR3 corresponding to the amino acid sequences of SEQ ID NO: 47 (HCDR1), SEQ ID NO: 50 (HCDR2), SEQ ID NO: 53 (HCDR3), and
a VL2 comprising a CDR1, CDR2 and CDR3 corresponding to the amino acid sequences of SEQ ID NO: 56 (LCDR1), SEQ ID NO: 59 (LCDR2), SEQ ID NO: 62 (LCDR3);
wherein the cytokine moiety is a variant IL-2;
and wherein all or part of the immunoglobulin Fc region or variant thereof binds to a human FcRn polypeptide.

46. The binding protein according to claim 45, wherein said first ABD has a Fab structure.

47. The binding protein according to claim 46, comprising three polypeptide chains (I), (II) and (III) that together comprise the first ABD and the second ABD:
V_{1A}-C_{1A}-Hinge₁-(Fc domain)_{A} (I)
V_{1B}-C_{1B}-Hinge₂-(Fc domain)_{B}-L₁-V_{2A}-C_{2A} (II)
V_{2B}-C_{2B}-Hinge₃-L₂-IL-2 (III)
wherein:
V_{1A} and V_{1B} form a binding pair V₁ (V_{H1}/V_{L1}) of the first ABD;
V_{2A} and V_{2B} form a binding pair V₂ (V_{H2}/V_{L2}) of the second ABD;
C_{1A} and C_{1B} form a pair C₁ (CH1/C_{L}) and C_{2A} and C_{2B} form a pair C₂ (CH1/C_{L});
wherein CH1 is an immunoglobulin heavy chain constant domain 1 and C_{L} is an immunoglobulin light chain constant domain;
Hinge₁, Hinge₂ and Hinge₃ are identical or different and correspond to all or part of an immunoglobulin hinge region, wherein Hinge₃ is optional;
(Fc domain)_{A} and (Fc domain)_{B} are identical or different, and comprise a CH2-CH3 domain;
L₁ and L₂ are an amino acid linker, wherein L₁ and L2 can be different or the same;
IL-2 is a variant human interleukin-2 polypeptide or portion thereof that binds to CD122 present on NK cells.

48. The binding protein of claim 45, comprising at least two polypeptide chains linked by at least one disulfide bridge.

49. The binding protein of claim 47, wherein:
polypeptide (I) consists of an amino acid sequence of SEQ ID NO: 1;
polypeptide (II) consists of an amino acid sequence of SEQ ID NO: 9; and
polypeptide (III) consists of an amino acid sequence of SEQ ID NO: 17.

50. The binding protein of claim 45, wherein said first ABD that binds to CD20 is a Fab and said second ABD that binds to NKp46 is an scFv.

51. The binding protein according to claim 50, wherein said second ABD and cytokine moiety have an arrangement:
-L1-V_{2A}-L2-V_{2B}-L3-IL-2,
wherein V_{2A} and V_{2B} form a binding pair V₂ (V_{H2}/V_{L2}) of the second ABD;
L₁, L₂ and L₃ are an amino acid linker, wherein L₁, L₂ and L₃ can be different or the same;
IL-2 is a variant human interleukin-2 polypeptide or portion thereof that binds to CD122 present on NK cells.

52. The binding protein of claim 45, wherein the variant IL-2 displays reduced binding to CD25 compared to a wild-type human IL-2 polypeptide.

53. The binding protein of claim 45, wherein the binding protein comprises a polypeptide comprising the amino acid sequence of SEQ ID NO: 1, and a polypeptide comprising the amino acid sequence of SEQ ID NO: 70.

54. A binding protein comprising a first and a second antigen binding domain (ABD), a cytokine moiety and all or part of an immunoglobulin Fc region or variant thereof, wherein each of said ABDs comprise an immunoglobulin heavy chain variable domain (VH) and an immunoglobulin light chain variable domain (VL), wherein each VH and VL comprises three complementary determining regions (CDR1, CDR2 and CDR3); and wherein:
(i) the first ABD binds specifically to human CD20 and comprises:
a VH1 comprising a CDR1, CDR2 and CDR3 corresponding to the amino acid sequences of SEQ ID NO: 29 (HCDR1), SEQ ID NO: 32 (HCDR2), SEQ ID NO: 35 (HCDR3), and
a VL1 comprising a CDR1, CDR2 and CDR3 corresponding to the amino acid sequences of SEQ ID NO: 38 (LCDR1), SEQ ID NO: 41 (LCDR2), SEQ ID NO: 44 (LCDR3);
(ii) the second ABD binds specifically to human NKp46 and comprises:
a VH2 comprising a CDR1, CDR2 and CDR3 corresponding to the amino acid sequences of SEQ ID NO: 47 (HCDR1), SEQ ID NO: 50 (HCDR2), SEQ ID NO: 53 (HCDR3), and
a VL2 comprising a CDR1, CDR2 and CDR3 corresponding to the amino acid sequences of SEQ ID NO: 56 (LCDR1), SEQ ID NO: 59 (LCDR2), SEQ ID NO: 62 (LCDR3);
wherein the cytokine moiety is a variant IL-2;
and wherein all or part of the immunoglobulin Fc region or variant thereof binds to a human FcRn polypeptide;
wherein the binding protein comprises two polypeptide chains (I) and (II):
V_{1A}-C_{1A}-Hinge₁-(Fc domain)_{A} (I)
V_{1B}-C_{1B}-Hinge₂-(Fc domain)_{B}-L₁-V_{2A}-L₂-V_{2B}-L₃-IL-2 (II)
wherein:
V_{1A} and V_{1B} form a binding pair V₁ (V_{H1}/V_{L1}) of the first ABD;
V_{2A} and V_{2B} form a binding pair V₂ (V_{H2}/V_{L2}) of the second ABD;
C_{1A} and C_{1B} form a pair C₁ (CH1/C_{L}) wherein CH1 is an immunoglobulin heavy chain constant domain 1 and C_{L} is an immunoglobulin light chain constant domain;
Hinge₁ and Hinge₂ are identical or different and correspond to all or part of an immunoglobulin hinge region;
(Fc domain)_{A} and (Fc domain)_{B} are identical or different, and comprise a CH2-CH3 domain;
L1, L2 and L3 are an amino acid linker, wherein L1, L2 and L3 can be different or the same;
IL-2 is a variant human interleukin-2 polypeptide or portion thereof that binds to CD122 present on NK cells.

55. The binding protein of claim 54, wherein V_{1A} is V_{L1} and V_{1B} is V_{H1}, and V_{2A} is V_{H2} and V_{2B} is V_{L2}.

56. The binding protein of claim 54, wherein:
(a) V_{H1} and V_{L1} corresponds to the amino acid sequences of SEQ ID NOS: 11 and 3 respectively, and/or
(b) V_{H2} and V_{L2} corresponds to the amino acid sequences of SEQ ID NOS: 93 and 95 respectively.

57. The binding protein of claim 54, wherein:
polypeptide (I) consists of the amino acid sequence of SEQ ID NO: 1; and
polypeptide (II) consists of the amino acid sequence of SEQ ID NO: 70.

58. A pharmaceutical composition comprising the binding protein according to claim 44 and a pharmaceutically acceptable carrier.

59. An isolated nucleic acid molecule comprising a nucleotide sequence that encodes the binding protein or a polypeptide chain thereof according to claim 44.

60. An expression vector comprising the nucleic acid molecule of claim 59.

61. An isolated cell comprising the nucleic acid molecule of claim 59.

62. A method of treating a disease and/or eliminating CD20-expressing cells in a subject, optionally wherein the disease is characterized by CD20-expressing cells, the method comprising administering to the subject a binding protein according to claim 44.

63. The method of claim 62, wherein said disease a B cell lymphoma Hodgkin's or non Hodgkin's B cell lymphoma, precursor B cell lymphoblastic leukemia/lymphoma and mature B cell neoplasms, B cell chronic lymphocytic leukemia (CLL)/small lymphocytic lymphoma (SLL), B cell prolymphocytic leukemia, lymphoplasmacytic lymphoma, mantle cell lymphoma (MCL), follicular lymphoma (FL), cutaneous follicle center lymphoma, marginal zone B cell lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenstrom's macroglobulinemia, and anaplastic large-cell lymphoma (ALCL).

64. The method of claim 62, wherein said disease is a mantle cell lymphoma.

65. The method of claim 62, wherein said disease is marginal zone B cell lymphoma.

66. The method of claim 62, wherein said disease is follicular lymphoma.

67. The method of claim 62, wherein the subject has a tumor characterized by low levels of cell surface expression of CD20, optionally wherein the level of CD20 is less than 100,000 copies of CD20 per cancer cell.

68. The method of claim 62, wherein the binding protein is used as a monotherapy.

69. A method of treatment comprising:
a) detecting cells in a sample obtained from the individual that express CD20, and
b) upon a determination that cells which express CD20 are comprised in the sample, optionally at a level corresponding at least to a reference level, administering to the individual a binding protein of claim 44.

70. A method of treating cancer in an individual, the method comprising:
a) detecting cell surface expression of NKG2D polypeptides on immune effector cells, optionally NK cells and/or T cells, in a sample from the individual, and
b) upon a determination of decreased cell surface expression of NKG2D polypeptides on immune effector cells, optionally compared to a reference level, administering to the individual a binding protein of claim 44.

71. A kit comprising a pharmaceutical composition containing a binding protein of claim 44 and instructions to allow a practitioner to administer the composition contained therein to a patient having a cancer.

72. A method for making a binding protein of claim 44, comprising a step of:
culturing host cell(s) under conditions suitable for expressing a plurality of recombinant polypeptides, said plurality comprising (i) a polypeptide comprising an amino acid sequence of SEQ ID NO: 1, 66, 68 or 77, and (ii) a polypeptide comprising an amino acid sequence of SEQ ID NO: 9, 67, 69, 70, 71, 72, 73, 75, 76, 78 or 79, and optionally (iii) a polypeptide comprising an amino acid sequence of SEQ ID NO: 17 or 74.


SEQUENCE LISTING (106 sequences)

SEQ ID NO: 1 - protein, 441 aa, Artificial Sequence
  synthesized
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 20 aa, Artificial Sequence
  synthesized
MSVPTQVLGLLLLWLTDARC

SEQ ID NO: 3 - protein, 107 aa, Artificial Sequence
  synthesized
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 4 - protein, 107 aa, Artificial Sequence [sequence omitted]

SEQ ID NO: 5 - protein, 10 aa, Artificial Sequence
  synthesized
DKTHTCPPCP

SEQ ID NO: 6 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 7 - protein, 110 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 8 - protein, 107 aa, Homo sapiens
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - protein, 682 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 10 - protein, 19 aa, Artificial Sequence
  synthesized
MEWSWVFLFFLSVTTGVHS

SEQ ID NO: 11 - protein, 122 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSS

SEQ ID NO: 12 - protein, 98 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRV

SEQ ID NO: 13 - protein, 15 aa, Artificial Sequence
  synthesized
EPKSCDKTHTCPPCP

SEQ ID NO: 14 - protein, 216 aa, Artificial Sequence
  synthesized
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 15 - protein, 4 aa, Artificial Sequence
  synthesized
STGS

SEQ ID NO: 16 - protein, 120 aa, Artificial Sequence
  synthesized
QVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSS

SEQ ID NO: 17 - protein, 357 aa, Artificial Sequence
  synthesized
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHSGSSSSGSSSSGSSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 18 - protein, 107 aa, Artificial Sequence
  synthesized
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIK

SEQ ID NO: 19 - protein, 10 aa, Artificial Sequence
  synthesized
EPKSCDKTHS

SEQ ID NO: 20 - protein, 5 aa, Artificial Sequence
  synthesized
GSSSS

SEQ ID NO: 21 - protein, 10 aa, Artificial Sequence
  synthesized
GSSSSGSSSS

SEQ ID NO: 22 - protein, 12 aa, Artificial Sequence
  synthesized
GSSSSGSSSSGS

SEQ ID NO: 23 - protein, 15 aa, Artificial Sequence
  synthesized
GSSSSGSSSSGSSSS

SEQ ID NO: 24 - protein, 133 aa, Artificial Sequence
  synthesized
APASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 25 - protein, 130 aa, Artificial Sequence
  synthesized
SSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 26 - protein, 130 aa, Artificial Sequence
  synthesized
SSSTKKTQLQLEHLLLDLQMILNGINNYKNPKLTAMLAKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 27 - protein, 133 aa, Homo sapiens [sequence omitted]

SEQ ID NO: 28 - protein, 130 aa, Homo sapiens
SSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 29 - protein, 5 aa, Artificial Sequence
  synthesized
DYAMH

SEQ ID NO: 30 - protein, 7 aa, Artificial Sequence
  synthesized
GFTFHDY

SEQ ID NO: 31 - protein, 8 aa, Artificial Sequence
  synthesized
GFTFHDYA

SEQ ID NO: 32 - protein, 17 aa, Artificial Sequence
  synthesized
TISWNSGTIGYADSVKG

SEQ ID NO: 33 - protein, 4 aa, Artificial Sequence
  synthesized
WNSG

SEQ ID NO: 34 - protein, 8 aa, Artificial Sequence
  synthesized
ISWNSGTI

SEQ ID NO: 35 - protein, 13 aa, Artificial Sequence
  synthesized
DIQYGNYYYGMDV

SEQ ID NO: 36 - protein, 11 aa, Artificial Sequence
  synthesized
IQYGNYYYGMD

SEQ ID NO: 37 - protein, 15 aa, Artificial Sequence
  synthesized
AKDIQYGNYYYGMDV

SEQ ID NO: 38 - protein, 11 aa, Artificial Sequence
  synthesized
RASQSVSSYLA

SEQ ID NO: 39 - protein, 7 aa, Artificial Sequence
  synthesized
SQSVSSY

SEQ ID NO: 40 - protein, 6 aa, Artificial Sequence
  synthesized
QSVSSY

SEQ ID NO: 41 - protein, 7 aa, Artificial Sequence
  synthesized
DASNRAT

SEQ ID NO: 42 - protein, 3 aa, Artificial Sequence
  synthesized
DAS

SEQ ID NO: 43 - protein, 3 aa, Artificial Sequence
  synthesized
DAS

SEQ ID NO: 44 - protein, 9 aa, Artificial Sequence
  synthesized
QQRSNWPIT

SEQ ID NO: 45 - protein, 6 aa, Artificial Sequence
  synthesized
RSNWPI

SEQ ID NO: 46 - protein, 9 aa, Artificial Sequence
  synthesized
QQRSNWPIT

SEQ ID NO: 47 - protein, 5 aa, Artificial Sequence
  synthesized
DYVIN

SEQ ID NO: 48 - protein, 7 aa, Artificial Sequence
  synthesized
GYTFSDY

SEQ ID NO: 49 - protein, 8 aa, Artificial Sequence
  synthesized
GYTFSDYV

SEQ ID NO: 50 - protein, 17 aa, Artificial Sequence
  synthesized
EIYPGSGTNYYNEKFKA

SEQ ID NO: 51 - protein, 6 aa, Artificial Sequence
  synthesized
YPGSGT

SEQ ID NO: 52 - protein, 8 aa, Artificial Sequence
  synthesized
IYPGSGTN

SEQ ID NO: 53 - protein, 11 aa, Artificial Sequence
  synthesized
RGRYGLYAMDY

SEQ ID NO: 54 - protein, 11 aa, Artificial Sequence
  synthesized
RGRYGLYAMDY

SEQ ID NO: 55 - protein, 13 aa, Artificial Sequence
  synthesized
ARRGRYGLYAMDY

SEQ ID NO: 56 - protein, 11 aa, Artificial Sequence
  synthesized
RASQDISNYLN

SEQ ID NO: 57 - protein, 11 aa, Artificial Sequence
  synthesized
RASQDISNYLN

SEQ ID NO: 58 - protein, 6 aa, Artificial Sequence
  synthesized
QDISNY

SEQ ID NO: 59 - protein, 7 aa, Artificial Sequence
  synthesized
YTSRLHS

SEQ ID NO: 60 - protein, 7 aa, Artificial Sequence
  synthesized
YTSRLHS

SEQ ID NO: 61 - protein, 3 aa, Artificial Sequence
  synthesized
YTS

SEQ ID NO: 62 - protein, 9 aa, Artificial Sequence
  synthesized
QQGNTRPWT

SEQ ID NO: 63 - protein, 9 aa, Artificial Sequence
  synthesized
QQGNTRPWT

SEQ ID NO: 64 - protein, 9 aa, Artificial Sequence
  synthesized
QQGNTRPWT

SEQ ID NO: 65 - protein, 130 aa, Artificial Sequence
  synthesized
SSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 66 - protein, 441 aa, Artificial Sequence
  synthesized
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 67 - protein, 682 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 68 - protein, 441 aa, Artificial Sequence
  synthesized
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - protein, 682 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 70 - protein, 842 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSVEGGSGGSGGSGGSGGVDDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKGSSSSGSSSSGSSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 71 - protein, 842 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSVEGGSGGSGGSGGSGGVDDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKGSSSSGSSSSGSSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 72 - protein, 842 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSVEGGSGGSGGSGGSGGVDDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKGSSSSGSSSSGSSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 73 - protein, 683 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 74 - protein, 356 aa, Artificial Sequence
  synthesized
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSSSSGSSSSGSSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFAQSIISTLT

SEQ ID NO: 75 - protein, 683 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 76 - protein, 683 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 77 - protein, 356 aa, Artificial Sequence
  synthesized
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGSSSSGSSSSGSSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 78 - protein, 587 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHS

SEQ ID NO: 79 - protein, 586 aa, Artificial Sequence
  synthesized
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 80 - protein, 121 aa, Artificial Sequence [sequence omitted]

SEQ ID NO: 81 - protein, 106 aa, Artificial Sequence [sequence omitted]

SEQ ID NO: 82 - protein, 119 aa, Artificial Sequence
  synthesized
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 83 - protein, 112 aa, Artificial Sequence
  synthesized
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIK

SEQ ID NO: 84 - protein, 122 aa, Artificial Sequence [sequence omitted]

SEQ ID NO: 85 - protein, 106 aa, Artificial Sequence [sequence omitted]

SEQ ID NO: 86 - protein, 121 aa, Artificial Sequence
  synthesized
QVQLVQSGAEVKKPGASVKVSCKASGYTFTSYNMHWVRQAPGQGLEWMGAIYPGNGDTSYNQKFQGRVTITADKSISTAYMELSSLRSEDTAVYYCARSTYYGGDWYFNVWGAGTLVTVSS

SEQ ID NO: 87 - protein, 106 aa, Artificial Sequence
  synthesized
QIVLTQSPSSLSASVGDRVTITCRASSSVSYIHWFQQKPGKSPKPLIYATSNLASGVPVRFSGSGSGTDYTLTISSLQPEDFATYYCQQWTSNPPTFGGGTKVEIK

SEQ ID NO: 88 - protein, 304 aa, Homo sapiens
MSSTLPALLCVGLCLSQRISAQQQTLPKPFIWAEPHFMVPKEKQVTICCQGNYGAVEYQLHFEGSLFAVDRPKPPERINKVKFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 89 - protein, 217 aa, Artificial Sequence
  synthesized
APELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 217 aa, Artificial Sequence
  synthesized
APEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 441 aa, Artificial Sequence
  synthesized
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 92 - protein, 441 aa, Artificial Sequence
  synthesized
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNRFTQKSLSLSPGK

SEQ ID NO: 93 - protein, 120 aa, Artificial Sequence; identical to SEQ ID NO: 16
  synthesized

SEQ ID NO: 94 - protein, 120 aa, Artificial Sequence
  synthesized
QVQLVQSGAEVKKPGSSVKVSCKASGYTFTDYVINWGRQAPGQGLEWIGEIYPGSGTNYYNEKFKAKATITADKSTSTAYMELSSLRSEDTAVYFCARRGRYGLYAMDYWGQGTTVTVSS

SEQ ID NO: 95 - protein, 107 aa, Artificial Sequence; identical to SEQ ID NO: 18
  synthesized

SEQ ID NO: 96 - protein, 18 aa, Artificial Sequence
  synthesized
GEGTSTGSGGSGGSGGAD

SEQ ID NO: 97 - protein, 700 aa, Artificial Sequence
  synthesized sequence
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSVEGGSGGSGGSGGSGGVDDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIK

SEQ ID NO: 98 - protein, 357 aa, Artificial Sequence
  synthesized sequence
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHSGSSSSGSSSSGSSSSTKKTQLQLEHLLLDLQMILNGINNYKNPKLTAMLAKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 99 - protein, 842 aa, Artificial Sequence
  synthesized sequence
EVQLVESGGGLVQPDRSLRLSCAASGFTFHDYAMHWVRQAPGKGLEWVSTISWNSGTIGYADSVKGRFTISRDNAKNSLYLQMNSLRAEDTALYYCAKDIQYGNYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSVEGGSGGSGGSGGSGGVDDIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKGSSSSGSSSSGSSSSTKKTQLQLEHLLLDLQMILNGINNYKNPKLTAMLTKKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 100 - protein, 356 aa, Artificial Sequence; identical to SEQ ID NO: 77
  synthesized sequence

SEQ ID NO: 101 - protein, 446 aa, Artificial Sequence
  synthesized sequence
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 679 aa, Artificial Sequence
  synthesized sequence
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 358 aa, Artificial Sequence
  synthesized sequence
DIQMTQSPSSLSASVGDRVTITCRASQDISNYLNWYQQKPGKAPKLLIYYTSRLHSGVPSRFSGSGSGTDFTFTISSLQPEDIATYFCQQGNTRPWTFGGGTKVEIKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHSGSSSSGSSSSAPASSSTKKTQLQLEHLLLDLQMILNGINNYKNPKLTRMLTAKFAMPKKATELKHLQCLEEELKPLEEVLNGAQSKNFHLRPRDLISNINVIVLELKGSETTFMCEYADETATIVEFLNRWITFAQSIISTLT

SEQ ID NO: 104 - protein, 446 aa, Artificial Sequence
  synthesized sequcne
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 679 aa, Artificial Sequence
  synthesized sequence
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYSSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGSTGSQVQLVQSGAEVKKPGSSVKVSCKASGYTFSDYVINWVRQAPGQGLEWMGEIYPGSGTNYYNEKFKAKATITADKSTSTAYMELSSLRSEDTAVYYCARRGRYGLYAMDYWGQGTTVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 358 aa, Artificial Sequence; identical to SEQ ID NO: 103
  synthesized